SEQUENCE LISTING (66 sequences)

SEQ ID NO: 1 - protein, 29 aa, Artificial Sequence
  Albumin binding domain; G418-GA3; ABD001
SDYYKNLINNAKTVEGVKALIDEILAALP

SEQ ID NO: 2 - protein, 29 aa, Artificial Sequence
  Albumin binding domain; ABD011
SDYYKNIINRAKTVEGVRALKLHILAALP

SEQ ID NO: 3 - protein, 29 aa, Artificial Sequence
  Albumin binding domain; ABD013
SDYYKNLINKAKTVEGVEALTLHILAALP

SEQ ID NO: 4 - protein, 29 aa, Artificial Sequence
  Albumin binding domain; ABD035
SDFYKRLINKAKTVEGVEALKLHILAALP

SEQ ID NO: 5 - protein, 428 aa, Artificial Sequence
  mCD63-Nanoluc
MAVEGGMKCVKFLLYVLLLAFCACAVGLIAIGVAVQVVLKQAMHITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGATRSNYTDWENIPGMAKDRVPDSCCINITVGCGNDFKESTIHTQGCVETIAIWLRKNILLVAAAALGIAFVEVLGIIFSCCLVKSIRSGYEVMGSGSGSGSGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAHHHHHH

SEQ ID NO: 6 - protein, 548 aa, Artificial Sequence
  mCD63-ABDX2-Nanoluc
MAVEGGMKCVKFLLYVLLLAFCACAVGLIAIGVAVQVVLKQAMHGSLAEAKVLANRELDKYGVSDFYKRLINKAKTVEGVEALKLHILAALPGSELMHITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGATRRTQHDEAVDANSLAEAKVLANRELDKYGVSDFYKRLINKAKTVEGVEALKLHILAALPRTCTSGTRSNYTDWENIPGMAKDRVPDSCCINITVGCGNDFKESTIHTQGCVETIAIWLRKNILLVAAAALGIAFVEVLGIIFSCCLVKSIRSGYEVMGSGSGSGSGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAHHHHHH

SEQ ID NO: 7 - protein, 482 aa, Artificial Sequence
  mCD63-ABD 1st Loop-Nanoluc
MAVEGGMKCVKFLLYVLLLAFCACAVGLIAIGVAVQVVLKQAMHGSLAEAKVLANRELDKYGVSDFYKRLINKAKTVEGVEALKLHILAALPGSELMHITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGATRSNYTDWENIPGMAKDRVPDSCCINITVGCGNDFKESTIHTQGCVETIAIWLRKNILLVAAAALGIAFVEVLGIIFSCCLVKSIRSGYEVMGSGSGSGSGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAHHHHHH

SEQ ID NO: 8 - protein, 494 aa, Artificial Sequence
  mCD63-ABD 2nd Loop-Nanoluc
MAVEGGMKCVKFLLYVLLLAFCACAVGLIAIGVAVQVVLKQAMHITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGATRRTQHDEAVDANSLAEAKVLANRELDKYGVSDFYKRLINKAKTVEGVEALKLHILAALPRTCTSGTRSNYTDWENIPGMAKDRVPDSCCINITVGCGNDFKESTIHTQGCVETIAIWLRKNILLVAAAALGIAFVEVLGIIFSCCLVKSIRSGYEVMGSGSGSGSGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAHHHHHH

SEQ ID NO: 9 - protein, 697 aa, Artificial Sequence
  mCD63-ABDX2-Neo-Nanoluc
MAVEGGMKCVKFLLYVLLLAFCACAVGLIAIGVAVQVVLKQAMHGSLAEAKVLANRELDKYGVSDFYKRLINKAKTVEGVEALKLHILAALPGSELREGVELCPGNKYEMRRHGTTHSLVIHDDSGSPFPAAVILRDALHMARGLKYLHQELMHITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGATRRTQHDEAVDANSLAEAKVLANRELDKYGVSDFYKRLINKAKTVEGVEALKLHILAALPRTCTSGVYDFFVWLGRGHLLGRLAAIVGKQVLLGRKVVVVRSHCHWNDLAVIPAGVVHNWDFEPRKVSCTPSKPSFQEFVDWENVSPELNSTDQPFLSGTRSNYTDWENIPGMAKDRVPDSCCINITVGCGNDFKESTIHTQGCVETIAIWLRKNILLVAAAALGIAFVEVLGIIFSCCLVKSIRSGYEVMGSGSGSGSGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAHHHHHH

SEQ ID NO: 10 - protein, 575 aa, Artificial Sequence
  mCD63-ABDX2-Mut30-Nanoluc
MAVEGGMKCVKFLLYVLLLAFCACAVGLIAIGVAVQVVLKQAMHGSLAEAKVLANRELDKYGVSDFYKRLINKAKTVEGVEALKLHILAALPGSELMHITHETTAGSLLPVVIIAVGAFLFLVAFVGCCGACKENYCLMITFAIFLSLIMLVEVAVAIAGYVFRDQVKSEFNKSFQQQMQNYLKDNKTATILDKLQKENNCCGATRRTQHDEAVDANSLAEAKVLANRELDKYGVSDFYKRLINKAKTVEGVEALKLHILAALPRTCTPSKPSFQEFVDWENVSPELNSTDQPFLSGTRSNYTDWENIPGMAKDRVPDSCCINITVGCGNDFKESTIHTQGCVETIAIWLRKNILLVAAAALGIAFVEVLGIIFSCCLVKSIRSGYEVMGSGSGSGSGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAHHHHHH

SEQ ID NO: 11 - protein, 402 aa, Artificial Sequence
  mCD9-Nluc
MPVKGGSKCIKYLLFGFNFIFWLAGIAVLAIGLWLRFDSQTKSMHIFEQENNHSSFYTGVYILIGAGALMMLVGFLGCCGAVQESQCMLGLFFGFLLVIFAIEIAAAVWGYTHKDEVIKELQEFYKDTYQKLRSKDEPQRETLKAIHMALDCCGITRAGPLEQFISDTCPKKQLLESFQVKPCPEAISEVFNNKFHIIGAVGIGIAVVMIFGMIFSMILCCAIRRSREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 12 - protein, 508 aa, Artificial Sequence
  mCD9-ABDX2-Nluc
MPVKGGSKCIKYLLFGFNFIFWLAGIAVLAIGLWLRFDSQTKSMHLAEAKVLANRELDKYGVSDFYKRLINKAKTVEGVEALKLHILAALPMHIFEQENNHSSFYTGVYILIGAGALMMLVGFLGCCGAVQESQCMLGLFFGFLLVIFAIEIAAAVWGYTHKDEVIKELQEFYKDTYQKLRSKDEPQRETLKAIHMALDCCGITRQHDEAVDANSLAEAKVLANRELDKYGVSDFYKRLINKAKTVEGVEALKLHILAALPTRAGPLEQFISDTCPKKQLLESFQVKPCPEAISEVFNNKFHIIGAVGIGIAVVMIFGMIFSMILCCAIRRSREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 13 - protein, 450 aa, Artificial Sequence
  mCD9-ABD 1st-Nluc
MPVKGGSKCIKYLLFGFNFIFWLAGIAVLAIGLWLRFDSQTKSMHLAEAKVLANRELDKYGVSDFYKRLINKAKTVEGVEALKLHILAALPMHIFEQENNHSSFYTGVYILIGAGALMMLVGFLGCCGAVQESQCMLGLFFGFLLVIFAIEIAAAVWGYTHKDEVIKELQEFYKDTYQKLRSKDEPQRETLKAIHMALDCCGITRAGPLEQFISDTCPKKQLLESFQVKPCPEAISEVFNNKFHIIGAVGIGIAVVMIFGMIFSMILCCAIRRSREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 14 - protein, 460 aa, Artificial Sequence
  mCD9-ABD 2nd-Nluc
MPVKGGSKCIKYLLFGFNFIFWLAGIAVLAIGLWLRFDSQTKSMHIFEQENNHSSFYTGVYILIGAGALMMLVGFLGCCGAVQESQCMLGLFFGFLLVIFAIEIAAAVWGYTHKDEVIKELQEFYKDTYQKLRSKDEPQRETLKAIHMALDCCGITRQHDEAVDANSLAEAKVLANRELDKYGVSDFYKRLINKAKTVEGVEALKLHILAALPTRAGPLEQFISDTCPKKQLLESFQVKPCPEAISEVFNNKFHIIGAVGIGIAVVMIFGMIFSMILCCAIRRSREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 15 - protein, 412 aa, Artificial Sequence
  mCD81-Nluc
MGVEGCTKCIKYLLFVFNFVFWLAGGVILGVALWLRHDPQTTSLLYMHLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSTRNALTTLTTTILRNSLCPSGGNILTPLLQQDCHQKIDELFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 16 - protein, 518 aa, Artificial Sequence
  mCD81-ABDX2-Nluc
MGVEGCTKCIKYLLFVFNFVFWLAGGVILGVALWLRHDPQTTSLLYMHLAEAKVLANRELDKYGVSDFYKRLINKAKTVEGVEALKLHILAALPMHLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSTRQHDEAVDANSLAEAKVLANRELDKYGVSDFYKRLINKAKTVEGVEALKLHILAALPTRNALTTLTTTILRNSLCPSGGNILTPLLQQDCHQKIDELFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 17 - protein, 460 aa, Artificial Sequence
  mCD81-ABD 1st-Nluc
MGVEGCTKCIKYLLFVFNFVFWLAGGVILGVALWLRHDPQTTSLLYMHLAEAKVLANRELDKYGVSDFYKRLINKAKTVEGVEALKLHILAALPMHLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSTRNALTTLTTTILRNSLCPSGGNILTPLLQQDCHQKIDELFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 18 - protein, 470 aa, Artificial Sequence
  mCD81-ABD 2nd-Nluc
MGVEGCTKCIKYLLFVFNFVFWLAGGVILGVALWLRHDPQTTSLLYMHLELGNKPAPNTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSTRQHDEAVDANSLAEAKVLANRELDKYGVSDFYKRLINKAKTVEGVEALKLHILAALPTRNALTTLTTTILRNSLCPSGGNILTPLLQQDCHQKIDELFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 19 - protein, 404 aa, Artificial Sequence
  hCD9-Nluc
MPVKGGTKCIKYLLFGFNFIFWLAGIAVLAIGLWLRFDSQTKSIMHFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLTRAGGVEQFISDICPKKDVLETFTVKSCPDAIKEVFDNKFHIIGAVGIGIAVVMIFGMIFSMILCCAIRRNREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 20 - protein, 510 aa, Artificial Sequence
  hCD9-ABDX2-Nluc
MPVKGGTKCIKYLLFGFNFIFWLAGIAVLAIGLWLRFDSQTKSIMHLAEAKVLANRELDKYGVSDFYKRLINKAKTVEGVEALKLHILAALPMHFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLTRQHDEAVDANSLAEAKVLANRELDKYGVSDFYKRLINKAKTVEGVEALKLHILAALPTRAGGVEQFISDICPKKDVLETFTVKSCPDAIKEVFDNKFHIIGAVGIGIAVVMIFGMIFSMILCCAIRRNREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 21 - protein, 452 aa, Artificial Sequence
  hCD9-ABD 1st-Nluc
MPVKGGTKCIKYLLFGFNFIFWLAGIAVLAIGLWLRFDSQTKSIMHLAEAKVLANRELDKYGVSDFYKRLINKAKTVEGVEALKLHILAALPMHFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLTRAGGVEQFISDICPKKDVLETFTVKSCPDAIKEVFDNKFHIIGAVGIGIAVVMIFGMIFSMILCCAIRRNREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 22 - protein, 462 aa, Artificial Sequence
  hCD9-ABD 2nd-Nluc
MPVKGGTKCIKYLLFGFNFIFWLAGIAVLAIGLWLRFDSQTKSIMHFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLTRQHDEAVDANSLAEAKVLANRELDKYGVSDFYKRLINKAKTVEGVEALKLHILAALPTRAGGVEQFISDICPKKDVLETFTVKSCPDAIKEVFDNKFHIIGAVGIGIAVVMIFGMIFSMILCCAIRRNREMVGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 23 - protein, 412 aa, Artificial Sequence
  hCD63-Nluc
MAVEGGMKCVKFLLYVLLLAFCACAVGLIAVGVGAQLVLSQTGTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAGSANYTDWEKIPSMSKNRVPDSCCINVTVGCGINFNEKAIHKEGCVEKIGGWLRKNVLVVAAAALGIAFVEVLGIVFACCLVKSIRSGYEVMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 24 - protein, 518 aa, Artificial Sequence
  hCD63-ABDX2-Nluc
MAVEGGMKCVKFLLYVLLLAFCACAVGLIAVGVGAQLVLSQTGTLAEAKVLANRELDKYGVSDFYKRLINKAKTVEGVEALKLHILAALPGTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAGSQHDEAVDANSLAEAKVLANRELDKYGVSDFYKRLINKAKTVEGVEALKLHILAALPGSANYTDWEKIPSMSKNRVPDSCCINVTVGCGINFNEKAIHKEGCVEKIGGWLRKNVLVVAAAALGIAFVEVLGIVFACCLVKSIRSGYEVMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 25 - protein, 460 aa, Artificial Sequence
  hCD63-ABD 1st-Nluc
MAVEGGMKCVKFLLYVLLLAFCACAVGLIAVGVGAQLVLSQTGTLAEAKVLANRELDKYGVSDFYKRLINKAKTVEGVEALKLHILAALPGTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAGSANYTDWEKIPSMSKNRVPDSCCINVTVGCGINFNEKAIHKEGCVEKIGGWLRKNVLVVAAAALGIAFVEVLGIVFACCLVKSIRSGYEVMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 26 - protein, 470 aa, Artificial Sequence
  hCD63-ABD 2nd-Nluc
MAVEGGMKCVKFLLYVLLLAFCACAVGLIAVGVGAQLVLSQTGTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAGSQHDEAVDANSLAEAKVLANRELDKYGVSDFYKRLINKAKTVEGVEALKLHILAALPGSANYTDWEKIPSMSKNRVPDSCCINVTVGCGINFNEKAIHKEGCVEKIGGWLRKNVLVVAAAALGIAFVEVLGIVFACCLVKSIRSGYEVMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 27 - protein, 470 aa, Artificial Sequence
  hCD63-RC 2nd-Nluc
MAVEGGMKCVKFLLYVLLLAFCACAVGLIAVGVGAQLVLSQTGTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAGSGKGCKYVKLKCFHPFDSFGLINKPFIKVTNPILIKFPISQYFSFGQAVSIYSLIMLGSANYTDWEKIPSMSKNRVPDSCCINVTVGCGINFNEKAIHKEGCVEKIGGWLRKNVLVVAAAALGIAFVEVLGIVFACCLVKSIRSGYEVMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 28 - protein, 412 aa, Artificial Sequence
  hCD81-Nluc
MGVEGCTKCIKYLLFVFNFVFWLAGGVILGVALWLRHDPQTTNLLYLEMHLGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSTRSTLTALTTSVLKNNLCPSGSNIISNLFKEDCHQKIDDLFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 29 - protein, 518 aa, Artificial Sequence
  hCD81-ABDX2-Nluc
MGVEGCTKCIKYLLFVFNFVFWLAGGVILGVALWLRHDPQTTNLLYLEMHLAEAKVLANRELDKYGVSDFYKRLINKAKTVEGVEALKLHILAALPMHLGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSTRQHDEAVDANSLAEAKVLANRELDKYGVSDFYKRLINKAKTVEGVEALKLHILAALPTRSTLTALTTSVLKNNLCPSGSNIISNLFKEDCHQKIDDLFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 30 - protein, 460 aa, Artificial Sequence
  hCD81-ABD 1st-Nluc
MGVEGCTKCIKYLLFVFNFVFWLAGGVILGVALWLRHDPQTTNLLYLEMHLAEAKVLANRELDKYGVSDFYKRLINKAKTVEGVEALKLHILAALPMHLGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSTRSTLTALTTSVLKNNLCPSGSNIISNLFKEDCHQKIDDLFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 31 - protein, 470 aa, Artificial Sequence
  hCD81-ABD 2nd-Nluc
MGVEGCTKCIKYLLFVFNFVFWLAGGVILGVALWLRHDPQTTNLLYLEMHLGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSTRQHDEAVDANSLAEAKVLANRELDKYGVSDFYKRLINKAKTVEGVEALKLHILAALPTRSTLTALTTSVLKNNLCPSGSNIISNLFKEDCHQKIDDLFSGKLYLIGIAAIVVAVIMIFEMILSMVLCCGIRNSSVYGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 32 - protein, 504 aa, Artificial Sequence
  Lamp2B-Nluc
MVCFRLFPVPGSGLVLVCLVLGAVRSYALKFETGGAPMHISQAVHAAHAEINEAGRSIINFEKLCTSVYDFFVWLGRGHLLGRLAAIVGKQVLLGRKVVVVRSHCHWNDLAVIPAGVVHNWDFEPRKVSPSKPSFQEFVDWENVSPELNSTDQPFLCTGSGSGSGSGSLKPAGILIPIIVGAGLSGLIIVIVIAYVIGRRKSYAGAQTLGGGGSYIPEAPRDGQAYVRKDGEWVLLSTFLSPANGTPTPTPTPTGRSLYPSLEDLKVDKVIQAQTAFSANPANPAILSEASAPIPHDGNLYPRLYPELSQYMGLSLEGGQLGTPTPTPTPTGGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 33 - protein, 562 aa, Artificial Sequence
  Lamp2B-ABD-Nluc
MVCFRLFPVPGSGLVLVCLVLGAVRSYALKFEQHDEAVDANSLAEAKVLANRELDKYGVSDFYKRLINKAKTVEGVEALKLHILAALPFETGGAPMHISQAVHAAHAEINEAGRSIINFEKLCTSVYDFFVWLGRGHLLGRLAAIVGKQVLLGRKVVVVRSHCHWNDLAVIPAGVVHNWDFEPRKVSPSKPSFQEFVDWENVSPELNSTDQPFLCTGSGSGSGSGSLKPAGILIPIIVGAGLSGLIIVIVIAYVIGRRKSYAGAQTLGGGGSYIPEAPRDGQAYVRKDGEWVLLSTFLSPANGTPTPTPTPTGRSLYPSLEDLKVDKVIQAQTAFSANPANPAILSEASAPIPHDGNLYPRLYPELSQYMGLSLEGGQLGTPTPTPTPTGGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 34 - DNA, 1287 bp, Artificial Sequence
  mCD63-Nanoluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgcacaccatcatcaccatcattga

SEQ ID NO: 35 - DNA, 1647 bp, Artificial Sequence
  mCD63-ABDX2-Nanoluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatggatccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggatccgagctcatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtcgtacgcagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttccccgtacgtgtacatccggaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgcacaccatcatcaccatcattga

SEQ ID NO: 36 - DNA, 1449 bp, Artificial Sequence
  mCD63-ABD 1st Loop-Nanoluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatggatccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggatccgagctcatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgcacaccatcatcaccatcattga

SEQ ID NO: 37 - DNA, 1485 bp, Artificial Sequence
  mCD63-ABD 2nd Loop-Nanoluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtcgtacgcagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttccccgtacgtgtacatccggaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgcacaccatcatcaccatcattga

SEQ ID NO: 38 - DNA, 2094 bp, Artificial Sequence
  mCD63-ABDX2-Neo-Nanoluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatggatccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggatccgagctcagggagggtgttgagctttgcccaggaaataaatatgaaatgcgacgacatggaacaacccattcattggtgattcacgatgactcaggaagtcctttcccagctgccgttatcttgagggacgcattgcatatggcccgggggctcaagtatttgcaccaggagctcatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtcgtacgcagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttccccgtacgtgtacatccggagtgtatgactttttcgtttggttgggacgaggacatcttcttggacggttggcagccatcgtggggaaacaagtactccttgggagaaaagtagtcgtagtgcggtctcattgccattggaacgacttggctgttatccccgccggcgtcgtccataactgggactttgagccccgaaaagtaagttgtacaccctcaaagcctagttttcaggaatttgtcgactgggaaaacgtatcacccgaacttaattctactgaccaacctttcctgtccggaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgcacaccatcatcaccatcattga

SEQ ID NO: 39 - DNA, 1728 bp, Artificial Sequence
  mCD63-ABDX2-Mut30-Nanoluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatggatccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggatccgagctcatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtcgtacgcagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttccccgtacgtgtacaccctcaaagcctagttttcaggaatttgtcgactgggaaaacgtatcacccgaacttaattctactgaccaacctttcctgtccggaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgcacaccatcatcaccatcattga

SEQ ID NO: 40 - DNA, 1206 bp, Artificial Sequence
  mCD9-Nluc
atgccagtgaaaggaggatctaagtgtataaaataccttctttttggattcaattttattttttggctggctggtatagcagtgctggcgataggtttgtggctccgattcgattcccagactaagagtatgcatattttcgaacaagaaaataaccatagcagtttctatactggggtgtatatcctcattggagctggcgctttgatgatgctggtggggttccttggttgctgcggcgctgttcaggagtcacaatgtatgctcgggctctttttcggcttcctccttgttatcttcgctatagaaattgccgctgccgtatggggttacactcacaaggatgaggttataaaagaactccaggaattttacaaagatacgtaccaaaagctcaggagcaaagacgaaccccagagagaaacccttaaagccatacatatggcacttgattgttgtggaataacgcgtgcaggaccacttgagcaatttatctctgacacttgtcccaaaaagcagctgctggaatcatttcaagttaagccctgcccggaggccattagtgaggtatttaataacaagttccacataattggggccgttgggatcgggatagccgtggttatgatttttggcatgatattctcaatgatactttgttgtgctatccgaaggtcacgagaaatggtaggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 41 - DNA, 1524 bp, Artificial Sequence
  mCD9-ABDX2-Nluc
atgccagtgaaaggaggatctaagtgtataaaataccttctttttggattcaattttattttttggctggctggtatagcagtgctggcgataggtttgtggctccgattcgattcccagactaagagtatgcatcttgctgaagctaaggtactggctaatcgcgagttggataaatatggtgtttcagacttttataagcgacttataaataaagccaagacagtggaaggagtagaggccctcaaattgcacatcttggccgccctccctatgcatattttcgaacaagaaaataaccatagcagtttctatactggggtgtatatcctcattggagctggcgctttgatgatgctggtggggttccttggttgctgcggcgctgttcaggagtcacaatgtatgctcgggctctttttcggcttcctccttgttatcttcgctatagaaattgccgctgccgtatggggttacactcacaaggatgaggttataaaagaactccaggaattttacaaagatacgtaccaaaagctcaggagcaaagacgaaccccagagagaaacccttaaagccatacatatggcacttgattgttgtggaataacgcgtcagcacgacgaggccgttgacgctaactcactggctgaggcgaaggttctcgctaatagagagctcgacaaatacggagtgtccgacttttacaaacgcctcattaacaaagccaagactgtcgaaggtgtagaggctctgaaactccacattttggcggctctcccaacgcgtgcaggaccacttgagcaatttatctctgacacttgtcccaaaaagcagctgctggaatcatttcaagttaagccctgcccggaggccattagtgaggtatttaataacaagttccacataattggggccgttgggatcgggatagccgtggttatgatttttggcatgatattctcaatgatactttgttgtgctatccgaaggtcacgagaaatggtaggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 42 - DNA, 1350 bp, Artificial Sequence
  mCD9-ABD 1st-Nluc
atgccagtgaaaggaggatctaagtgtataaaataccttctttttggattcaattttattttttggctggctggtatagcagtgctggcgataggtttgtggctccgattcgattcccagactaagagtatgcatcttgctgaagctaaggtactggctaatcgcgagttggataaatatggtgtttcagacttttataagcgacttataaataaagccaagacagtggaaggagtagaggccctcaaattgcacatcttggccgccctccctatgcatattttcgaacaagaaaataaccatagcagtttctatactggggtgtatatcctcattggagctggcgctttgatgatgctggtggggttccttggttgctgcggcgctgttcaggagtcacaatgtatgctcgggctctttttcggcttcctccttgttatcttcgctatagaaattgccgctgccgtatggggttacactcacaaggatgaggttataaaagaactccaggaattttacaaagatacgtaccaaaagctcaggagcaaagacgaaccccagagagaaacccttaaagccatacatatggcacttgattgttgtggaataacgcgtgcaggaccacttgagcaatttatctctgacacttgtcccaaaaagcagctgctggaatcatttcaagttaagccctgcccggaggccattagtgaggtatttaataacaagttccacataattggggccgttgggatcgggatagccgtggttatgatttttggcatgatattctcaatgatactttgttgtgctatccgaaggtcacgagaaatggtaggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 43 - DNA, 1380 bp, Artificial Sequence
  mCD9-ABD 2nd-Nluc
atgccagtgaaaggaggatctaagtgtataaaataccttctttttggattcaattttattttttggctggctggtatagcagtgctggcgataggtttgtggctccgattcgattcccagactaagagtatgcatattttcgaacaagaaaataaccatagcagtttctatactggggtgtatatcctcattggagctggcgctttgatgatgctggtggggttccttggttgctgcggcgctgttcaggagtcacaatgtatgctcgggctctttttcggcttcctccttgttatcttcgctatagaaattgccgctgccgtatggggttacactcacaaggatgaggttataaaagaactccaggaattttacaaagatacgtaccaaaagctcaggagcaaagacgaaccccagagagaaacccttaaagccatacatatggcacttgattgttgtggaataacgcgtcagcacgacgaggccgttgacgctaactcactggctgaggcgaaggttctcgctaatagagagctcgacaaatacggagtgtccgacttttacaaacgcctcattaacaaagccaagactgtcgaaggtgtagaggctctgaaactccacattttggcggctctcccaacgcgtgcaggaccacttgagcaatttatctctgacacttgtcccaaaaagcagctgctggaatcatttcaagttaagccctgcccggaggccattagtgaggtatttaataacaagttccacataattggggccgttgggatcgggatagccgtggttatgatttttggcatgatattctcaatgatactttgttgtgctatccgaaggtcacgagaaatggtaggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 44 - DNA, 1266 bp, Artificial Sequence
  mCD63-Nluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 45 - DNA, 1626 bp, Artificial Sequence
  mCD63-ABDX2-Nluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatggatccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggatccgagctcatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtcgtacgcagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttccccgtacgtgtacatccggaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 46 - DNA, 1428 bp, Artificial Sequence
  mCD63-ABD 1st-Nluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatggatccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggatccgagctcatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 47 - DNA, 1464 bp, Artificial Sequence
  mCD63-ABD 2nd-Nluc
atggccgtcgaggggggtatgaagtgtgtgaaatttcttctttatgtgcttttgctggctttctgcgcatgcgccgtaggattgattgccataggggttgccgttcaagttgtactcaaacaagccatgcatattacacatgaaaccacagccggtagccttctcccagtggtgataatagccgttggtgccttccttttccttgtagccttcgtgggttgttgtggcgcttgtaaggagaattactgtctgatgatcacttttgctatttttctcagtttgatcatgttggtggaggttgccgtagcaatagcaggctacgtcttcagagaccaggtaaaaagcgagtttaataagtcattccagcagcagatgcaaaactacttgaaagataacaaaacagcaactatcctggacaagctgcagaaagagaacaattgctgtggggcaacgcgtcgtacgcagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttccccgtacgtgtacatccggaacgcgtagtaactacaccgattgggaaaacatacccggaatggcaaaagaccgcgtacccgattcttgttgtattaatattaccgtgggctgtgggaacgacttcaaagaatctaccatccatactcagggttgcgtcgagaccatcgccatctggctcaggaagaatatccttttggtcgcagcagctgctttgggcatcgcttttgtggaagtactcgggataatattcagctgctgccttgtgaaaagcatccgaagcggctatgaagtcatgggttcaggatcagggtcaggaagcggcagtgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 48 - DNA, 1236 bp, Artificial Sequence
  mCD81-Nluc
atgggggtggaaggatgcactaagtgtataaaatatctgttgtttgtgtttaattttgtcttctggctcgccggtggcgtcatattgggagtcgcattgtggctcaggcatgatccgcagaccacttcacttttgtacatgcatctggagctcggaaataagcccgcaccgaacacgttctacgttggaatttatatactcattgctgtaggagctgtcatgatgtttgttgggtttttgggatgctatggagctatacaggagtctcaatgtcttttgggcaccttctttacgtgtttggtgattttgttcgcctgtgaagttgctgcaggtatctggggctttgtaaataaggaccaaatagccaaggacgtgaaacaattctacgaccaagcactgcagcaagcggtaatggacgacgacgcaaataatgccaaagctgtcgtcaagacatttcatgaaacgcttaactgctgcggaagcacgcgtaatgcgctcaccacactgactactaccatacttaggaacagcctgtgcccttcaggaggaaatatacttactcccttgttgcagcaagactgtcatcaaaaaattgacgaacttttctcaggcaaactttacctgattgggattgctgctattgtggtagctgtaataatgatattcgaaatgattctgagcatggtgttgtgctgtggtattcggaactctagcgtgtatggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 49 - DNA, 1554 bp, Artificial Sequence
  mCD81-ABDX2-Nluc
atgggggtggaaggatgcactaagtgtataaaatatctgttgtttgtgtttaattttgtcttctggctcgccggtggcgtcatattgggagtcgcattgtggctcaggcatgatccgcagaccacttcacttttgtacatgcatctggcggaggcgaaggtcctggccaatcgagagcttgataagtatggtgtatcagatttctacaaaagactcattaataaagcaaagacggtcgaaggagtagaagctctcaaacttcacattctcgctgcactgccgatgcatctggagctcggaaataagcccgcaccgaacacgttctacgttggaatttatatactcattgctgtaggagctgtcatgatgtttgttgggtttttgggatgctatggagctatacaggagtctcaatgtcttttgggcaccttctttacgtgtttggtgattttgttcgcctgtgaagttgctgcaggtatctggggctttgtaaataaggaccaaatagccaaggacgtgaaacaattctacgaccaagcactgcagcaagcggtaatggacgacgacgcaaataatgccaaagctgtcgtcaagacatttcatgaaacgcttaactgctgcggaagcacgcgtcaacacgatgaagccgtggacgcaaacagcctcgcagaagcgaaggtcctcgctaacagggagcttgataaatacggtgtttccgatttctataagcgactcattaacaaggccaaaactgtagaaggcgtggaggcgcttaaattgcatatattggcagccttgccaacgcgtaatgcgctcaccacactgactactaccatacttaggaacagcctgtgcccttcaggaggaaatatacttactcccttgttgcagcaagactgtcatcaaaaaattgacgaacttttctcaggcaaactttacctgattgggattgctgctattgtggtagctgtaataatgatattcgaaatgattctgagcatggtgttgtgctgtggtattcggaactctagcgtgtatggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 50 - DNA, 1380 bp, Artificial Sequence
  mCD81-ABD 1st-Nluc
atgggggtggaaggatgcactaagtgtataaaatatctgttgtttgtgtttaattttgtcttctggctcgccggtggcgtcatattgggagtcgcattgtggctcaggcatgatccgcagaccacttcacttttgtacatgcatctggcggaggcgaaggtcctggccaatcgagagcttgataagtatggtgtatcagatttctacaaaagactcattaataaagcaaagacggtcgaaggagtagaagctctcaaacttcacattctcgctgcactgccgatgcatctggagctcggaaataagcccgcaccgaacacgttctacgttggaatttatatactcattgctgtaggagctgtcatgatgtttgttgggtttttgggatgctatggagctatacaggagtctcaatgtcttttgggcaccttctttacgtgtttggtgattttgttcgcctgtgaagttgctgcaggtatctggggctttgtaaataaggaccaaatagccaaggacgtgaaacaattctacgaccaagcactgcagcaagcggtaatggacgacgacgcaaataatgccaaagctgtcgtcaagacatttcatgaaacgcttaactgctgcggaagcacgcgtaatgcgctcaccacactgactactaccatacttaggaacagcctgtgcccttcaggaggaaatatacttactcccttgttgcagcaagactgtcatcaaaaaattgacgaacttttctcaggcaaactttacctgattgggattgctgctattgtggtagctgtaataatgatattcgaaatgattctgagcatggtgttgtgctgtggtattcggaactctagcgtgtatggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 51 - DNA, 1410 bp, Artificial Sequence
  mCD81-ABD 2nd-Nluc
atgggggtggaaggatgcactaagtgtataaaatatctgttgtttgtgtttaattttgtcttctggctcgccggtggcgtcatattgggagtcgcattgtggctcaggcatgatccgcagaccacttcacttttgtacatgcatctggagctcggaaataagcccgcaccgaacacgttctacgttggaatttatatactcattgctgtaggagctgtcatgatgtttgttgggtttttgggatgctatggagctatacaggagtctcaatgtcttttgggcaccttctttacgtgtttggtgattttgttcgcctgtgaagttgctgcaggtatctggggctttgtaaataaggaccaaatagccaaggacgtgaaacaattctacgaccaagcactgcagcaagcggtaatggacgacgacgcaaataatgccaaagctgtcgtcaagacatttcatgaaacgcttaactgctgcggaagcacgcgtcaacacgatgaagccgtggacgcaaacagcctcgcagaagcgaaggtcctcgctaacagggagcttgataaatacggtgtttccgatttctataagcgactcattaacaaggccaaaactgtagaaggcgtggaggcgcttaaattgcatatattggcagccttgccaacgcgtaatgcgctcaccacactgactactaccatacttaggaacagcctgtgcccttcaggaggaaatatacttactcccttgttgcagcaagactgtcatcaaaaaattgacgaacttttctcaggcaaactttacctgattgggattgctgctattgtggtagctgtaataatgatattcgaaatgattctgagcatggtgttgtgctgtggtattcggaactctagcgtgtatggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 52 - DNA, 1212 bp, Artificial Sequence
  hCD9-Nluc
atgcccgtcaagggcgggactaagtgtataaagtacttgctttttggtttcaatttcatcttctggcttgcgggaatcgcggtgcttgccattggactttggctgagattcgactcccagacaaagtccattatgcattttgaacaagagactaacaataataacagcagcttttataccggggtgtacatactgatcggcgccggagcattgatgatgttggtaggtttcctgggttgctgtggggctgttcaggaatctcaatgtatgctggggttgttctttgggttcctgctggtaatttttgctatagaaattgccgccgcgatttggggctattcacacaaggatgaggttattaaagaggtccaagaattttataaagatacctataacaaactgaaaacgaaggatgaaccacaacgggagacccttaaggcaatacactatgcgcttaactgttgcggcttgacgcgtgcagggggagtagagcagttcatcagtgatatatgtccaaaaaaagacgtacttgaaaccttcacggtcaaatcatgcccggatgccattaaggaggtgtttgataacaaattccacattataggggcggtgggtatcggcatagccgtcgtaatgatcttcgggatgatcttcagcatgatcctctgctgcgcgatccggcgaaaccgagaaatggttggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 53 - DNA, 1530 bp, Artificial Sequence
  hCD9-ABDX2-Nluc
atgcccgtcaagggcgggactaagtgtataaagtacttgctttttggtttcaatttcatcttctggcttgcgggaatcgcggtgcttgccattggactttggctgagattcgactcccagacaaagtccattatgcatctggctgaagctaaagtcctggctaatcgcgaattggacaagtatggtgtttctgacttttacaagaggctgattaataaagcgaagacggttgagggagtggaagccctcaagctccatatactcgcggctctgccaatgcattttgaacaagagactaacaataataacagcagcttttataccggggtgtacatactgatcggcgccggagcattgatgatgttggtaggtttcctgggttgctgtggggctgttcaggaatctcaatgtatgctggggttgttctttgggttcctgctggtaatttttgctatagaaattgccgccgcgatttggggctattcacacaaggatgaggttattaaagaggtccaagaattttataaagatacctataacaaactgaaaacgaaggatgaaccacaacgggagacccttaaggcaatacactatgcgcttaactgttgcggcttgacgcgtcaacacgatgaagctgtggatgccaatagccttgccgaggccaaggttcttgcgaacagggagcttgataagtatggggtgtctgatttttacaaacggctcattaacaaagcgaagacagttgagggtgtagaagcacttaaattgcacattttggcggcgctgccaacgcgtgcagggggagtagagcagttcatcagtgatatatgtccaaaaaaagacgtacttgaaaccttcacggtcaaatcatgcccggatgccattaaggaggtgtttgataacaaattccacattataggggcggtgggtatcggcatagccgtcgtaatgatcttcgggatgatcttcagcatgatcctctgctgcgcgatccggcgaaaccgagaaatggttggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 54 - DNA, 1356 bp, Artificial Sequence
  hCD9-ABD 1st-Nluc
atgcccgtcaagggcgggactaagtgtataaagtacttgctttttggtttcaatttcatcttctggcttgcgggaatcgcggtgcttgccattggactttggctgagattcgactcccagacaaagtccattatgcatctggctgaagctaaagtcctggctaatcgcgaattggacaagtatggtgtttctgacttttacaagaggctgattaataaagcgaagacggttgagggagtggaagccctcaagctccatatactcgcggctctgccaatgcattttgaacaagagactaacaataataacagcagcttttataccggggtgtacatactgatcggcgccggagcattgatgatgttggtaggtttcctgggttgctgtggggctgttcaggaatctcaatgtatgctggggttgttctttgggttcctgctggtaatttttgctatagaaattgccgccgcgatttggggctattcacacaaggatgaggttattaaagaggtccaagaattttataaagatacctataacaaactgaaaacgaaggatgaaccacaacgggagacccttaaggcaatacactatgcgcttaactgttgcggcttgacgcgtgcagggggagtagagcagttcatcagtgatatatgtccaaaaaaagacgtacttgaaaccttcacggtcaaatcatgcccggatgccattaaggaggtgtttgataacaaattccacattataggggcggtgggtatcggcatagccgtcgtaatgatcttcgggatgatcttcagcatgatcctctgctgcgcgatccggcgaaaccgagaaatggttggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 55 - DNA, 1386 bp, Artificial Sequence
  hCD9-ABD 2nd-Nluc
atgcccgtcaagggcgggactaagtgtataaagtacttgctttttggtttcaatttcatcttctggcttgcgggaatcgcggtgcttgccattggactttggctgagattcgactcccagacaaagtccattatgcattttgaacaagagactaacaataataacagcagcttttataccggggtgtacatactgatcggcgccggagcattgatgatgttggtaggtttcctgggttgctgtggggctgttcaggaatctcaatgtatgctggggttgttctttgggttcctgctggtaatttttgctatagaaattgccgccgcgatttggggctattcacacaaggatgaggttattaaagaggtccaagaattttataaagatacctataacaaactgaaaacgaaggatgaaccacaacgggagacccttaaggcaatacactatgcgcttaactgttgcggcttgacgcgtcaacacgatgaagctgtggatgccaatagccttgccgaggccaaggttcttgcgaacagggagcttgataagtatggggtgtctgatttttacaaacggctcattaacaaagcgaagacagttgagggtgtagaagcacttaaattgcacattttggcggcgctgccaacgcgtgcagggggagtagagcagttcatcagtgatatatgtccaaaaaaagacgtacttgaaaccttcacggtcaaatcatgcccggatgccattaaggaggtgtttgataacaaattccacattataggggcggtgggtatcggcatagccgtcgtaatgatcttcgggatgatcttcagcatgatcctctgctgcgcgatccggcgaaaccgagaaatggttggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 56 - DNA, 1236 bp, Artificial Sequence
  hCD63-Nluc
atggcggtggagggaggcatgaagtgcgtcaaatttctgttgtatgtcttgctgctcgccttctgcgcgtgcgctgtgggtctcatagccgttggagtcggagcccagctcgtcttgtctcagacgggtaccataatccaaggtgcaaccccggggtccctgcttccagtagtgatcattgcagtcggtgttttcctgttcttggtggctttcgtaggctgttgtggcgcttgcaaggaaaactattgtcttatgatcacgttcgctatattcctgagccttattatgctcgtggaagttgcggcggcaatcgcaggttacgtttttcgcgacaaagtgatgtccgaatttaacaacaacttccgacagcagatggagaattaccctaagaataaccatacagcatccatcctcgaccggatgcaggcggattttaaatgctgcggggcaggatccgctaactatacggattgggagaagataccatctatgtcaaaaaatcgcgtcccggacagctgttgcatcaatgttactgtaggctgtggtatcaatttcaatgaaaaagctatacataaggaaggctgcgtagaaaaaataggtggctggcttcgcaaaaacgtgcttgtggtggctgcggcagccctcggcattgcatttgtggaagttctcgggatagtatttgcatgctgccttgttaaaagtatccgaagtggatacgaagttatggtattcacacttgaggactttgtgggggattggcgacagactgctggctataatcttgaccaagttctggaacaggggggcgtcagcagtctctttcaaaatcttggcgtcagcgttaccccgatacaacgaatcgtactttctggggaaaatggacttaagatcgacatccatgttataatcccttacgaagggttgtccggcgatcagatggggcagattgaaaagatattcaaggtcgtatatccagtcgacgatcatcactttaaagtgatactccattatgggactctggtcattgacggtgtaactccgaacatgattgattatttcggcaggccctatgaggggattgctgtttttgatggcaaaaagatcaccgtcactggcacattgtggaatggtaacaaaatcattgacgaacggcttatcaatccggatggaagcttgcttttccgcgttacgattaatggtgttactggctggcgcctctgtgaacggattctggca

SEQ ID NO: 57 - DNA, 1554 bp, Artificial Sequence
  hCD63-ABDX2-Nluc
atggcggtggagggaggcatgaagtgcgtcaaatttctgttgtatgtcttgctgctcgccttctgcgcgtgcgctgtgggtctcatagccgttggagtcggagcccagctcgtcttgtctcagacgggtaccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggtaccataatccaaggtgcaaccccggggtccctgcttccagtagtgatcattgcagtcggtgttttcctgttcttggtggctttcgtaggctgttgtggcgcttgcaaggaaaactattgtcttatgatcacgttcgctatattcctgagccttattatgctcgtggaagttgcggcggcaatcgcaggttacgtttttcgcgacaaagtgatgtccgaatttaacaacaacttccgacagcagatggagaattaccctaagaataaccatacagcatccatcctcgaccggatgcaggcggattttaaatgctgcggggcaggatcccagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttcccggatccgctaactatacggattgggagaagataccatctatgtcaaaaaatcgcgtcccggacagctgttgcatcaatgttactgtaggctgtggtatcaatttcaatgaaaaagctatacataaggaaggctgcgtagaaaaaataggtggctggcttcgcaaaaacgtgcttgtggtggctgcggcagccctcggcattgcatttgtggaagttctcgggatagtatttgcatgctgccttgttaaaagtatccgaagtggatacgaagttatggtattcacacttgaggactttgtgggggattggcgacagactgctggctataatcttgaccaagttctggaacaggggggcgtcagcagtctctttcaaaatcttggcgtcagcgttaccccgatacaacgaatcgtactttctggggaaaatggacttaagatcgacatccatgttataatcccttacgaagggttgtccggcgatcagatggggcagattgaaaagatattcaaggtcgtatatccagtcgacgatcatcactttaaagtgatactccattatgggactctggtcattgacggtgtaactccgaacatgattgattatttcggcaggccctatgaggggattgctgtttttgatggcaaaaagatcaccgtcactggcacattgtggaatggtaacaaaatcattgacgaacggcttatcaatccggatggaagcttgcttttccgcgttacgattaatggtgttactggctggcgcctctgtgaacggattctggca

SEQ ID NO: 58 - DNA, 1380 bp, Artificial Sequence
  hCD63-ABD 1st-Nluc
atggcggtggagggaggcatgaagtgcgtcaaatttctgttgtatgtcttgctgctcgccttctgcgcgtgcgctgtgggtctcatagccgttggagtcggagcccagctcgtcttgtctcagacgggtaccttggcagaagcaaaggtgctcgccaaccgagaactcgataagtacggcgtctctgatttttataaaagactgataaataaggctaaaaccgtggagggggtggaagccttgaagcttcacatcctcgcagcccttcctggtaccataatccaaggtgcaaccccggggtccctgcttccagtagtgatcattgcagtcggtgttttcctgttcttggtggctttcgtaggctgttgtggcgcttgcaaggaaaactattgtcttatgatcacgttcgctatattcctgagccttattatgctcgtggaagttgcggcggcaatcgcaggttacgtttttcgcgacaaagtgatgtccgaatttaacaacaacttccgacagcagatggagaattaccctaagaataaccatacagcatccatcctcgaccggatgcaggcggattttaaatgctgcggggcaggatccgctaactatacggattgggagaagataccatctatgtcaaaaaatcgcgtcccggacagctgttgcatcaatgttactgtaggctgtggtatcaatttcaatgaaaaagctatacataaggaaggctgcgtagaaaaaataggtggctggcttcgcaaaaacgtgcttgtggtggctgcggcagccctcggcattgcatttgtggaagttctcgggatagtatttgcatgctgccttgttaaaagtatccgaagtggatacgaagttatggtattcacacttgaggactttgtgggggattggcgacagactgctggctataatcttgaccaagttctggaacaggggggcgtcagcagtctctttcaaaatcttggcgtcagcgttaccccgatacaacgaatcgtactttctggggaaaatggacttaagatcgacatccatgttataatcccttacgaagggttgtccggcgatcagatggggcagattgaaaagatattcaaggtcgtatatccagtcgacgatcatcactttaaagtgatactccattatgggactctggtcattgacggtgtaactccgaacatgattgattatttcggcaggccctatgaggggattgctgtttttgatggcaaaaagatcaccgtcactggcacattgtggaatggtaacaaaatcattgacgaacggcttatcaatccggatggaagcttgcttttccgcgttacgattaatggtgttactggctggcgcctctgtgaacggattctggca

SEQ ID NO: 59 - DNA, 1410 bp, Artificial Sequence
  hCD63-ABD 2nd-Nluc
atggcggtggagggaggcatgaagtgcgtcaaatttctgttgtatgtcttgctgctcgccttctgcgcgtgcgctgtgggtctcatagccgttggagtcggagcccagctcgtcttgtctcagacgggtaccataatccaaggtgcaaccccggggtccctgcttccagtagtgatcattgcagtcggtgttttcctgttcttggtggctttcgtaggctgttgtggcgcttgcaaggaaaactattgtcttatgatcacgttcgctatattcctgagccttattatgctcgtggaagttgcggcggcaatcgcaggttacgtttttcgcgacaaagtgatgtccgaatttaacaacaacttccgacagcagatggagaattaccctaagaataaccatacagcatccatcctcgaccggatgcaggcggattttaaatgctgcggggcaggatcccagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttcccggatccgctaactatacggattgggagaagataccatctatgtcaaaaaatcgcgtcccggacagctgttgcatcaatgttactgtaggctgtggtatcaatttcaatgaaaaagctatacataaggaaggctgcgtagaaaaaataggtggctggcttcgcaaaaacgtgcttgtggtggctgcggcagccctcggcattgcatttgtggaagttctcgggatagtatttgcatgctgccttgttaaaagtatccgaagtggatacgaagttatggtattcacacttgaggactttgtgggggattggcgacagactgctggctataatcttgaccaagttctggaacaggggggcgtcagcagtctctttcaaaatcttggcgtcagcgttaccccgatacaacgaatcgtactttctggggaaaatggacttaagatcgacatccatgttataatcccttacgaagggttgtccggcgatcagatggggcagattgaaaagatattcaaggtcgtatatccagtcgacgatcatcactttaaagtgatactccattatgggactctggtcattgacggtgtaactccgaacatgattgattatttcggcaggccctatgaggggattgctgtttttgatggcaaaaagatcaccgtcactggcacattgtggaatggtaacaaaatcattgacgaacggcttatcaatccggatggaagcttgcttttccgcgttacgattaatggtgttactggctggcgcctctgtgaacggattctggca

SEQ ID NO: 60 - DNA, 1410 bp, Artificial Sequence
  hCD63-RC 2nd-Nluc
atggcggtggagggaggcatgaagtgcgtcaaatttctgttgtatgtcttgctgctcgccttctgcgcgtgcgctgtgggtctcatagccgttggagtcggagcccagctcgtcttgtctcagacgggtaccataatccaaggtgcaaccccggggtccctgcttccagtagtgatcattgcagtcggtgttttcctgttcttggtggctttcgtaggctgttgtggcgcttgcaaggaaaactattgtcttatgatcacgttcgctatattcctgagccttattatgctcgtggaagttgcggcggcaatcgcaggttacgtttttcgcgacaaagtgatgtccgaatttaacaacaacttccgacagcagatggagaattaccctaagaataaccatacagcatccatcctcgaccggatgcaggcggattttaaatgctgcggggcaggatccgggaagggctgcaagtatgtgaagcttaagtgcttccaccccttcgacagttttggccttattaataagccgtttataaaagtcactaaccccatacttatcaagttccctattagccaatactttagcttcggccaagctgttagcatctacagcctcatcatgctgggatccgctaactatacggattgggagaagataccatctatgtcaaaaaatcgcgtcccggacagctgttgcatcaatgttactgtaggctgtggtatcaatttcaatgaaaaagctatacataaggaaggctgcgtagaaaaaataggtggctggcttcgcaaaaacgtgcttgtggtggctgcggcagccctcggcattgcatttgtggaagttctcgggatagtatttgcatgctgccttgttaaaagtatccgaagtggatacgaagttatggtattcacacttgaggactttgtgggggattggcgacagactgctggctataatcttgaccaagttctggaacaggggggcgtcagcagtctctttcaaaatcttggcgtcagcgttaccccgatacaacgaatcgtactttctggggaaaatggacttaagatcgacatccatgttataatcccttacgaagggttgtccggcgatcagatggggcagattgaaaagatattcaaggtcgtatatccagtcgacgatcatcactttaaagtgatactccattatgggactctggtcattgacggtgtaactccgaacatgattgattatttcggcaggccctatgaggggattgctgtttttgatggcaaaaagatcaccgtcactggcacattgtggaatggtaacaaaatcattgacgaacggcttatcaatccggatggaagcttgcttttccgcgttacgattaatggtgttactggctggcgcctctgtgaacggattctggca

SEQ ID NO: 61 - DNA, 1236 bp, Artificial Sequence
  hCD81-Nluc
atgggagtcgaaggatgcacgaagtgcatcaaatacctcttgtttgtttttaattttgtcttctggttggccggcggtgtaatccttggtgtagctctttggcttaggcatgatccgcaaactactaatttgctttatttggagatgcatctcggtgataaacctgctcccaatactttctatgtcggtatttatattctgattgcagttggcgcagttatgatgtttgttggtttccttgggtgttacggtgcgatacaagagtcacagtgcctccttggtactttttttacatgtctggtaatattgttcgcttgtgaggttgcagcagggatctggggttttgtaaacaaggatcaaattgcaaaagatgtcaaacaattttacgaccaagccctgcaacaagcagtggtggacgacgatgctaataacgctaaggctgtagtaaaaacttttcatgaaactctggactgttgtggatcaacgcgttctacgcttacggcactcacaacttctgtattgaaaaataacttgtgtcccagcggtagcaacattataagcaaccttttcaaggaggactgccaccaaaaaattgacgacctgtttagcgggaagctctatctcatcgggatcgcggctattgtagtggcagtcataatgatatttgaaatgatactgtccatggttctgtgttgtggcattagaaacagttctgtttacggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 62 - DNA, 1554 bp, Artificial Sequence
  hCD81-ABDX2-Nluc
atgggagtcgaaggatgcacgaagtgcatcaaatacctcttgtttgtttttaattttgtcttctggttggccggcggtgtaatccttggtgtagctctttggcttaggcatgatccgcaaactactaatttgctttatttggagatgcatcttgcagaagccaaggtccttgcgaatagagaactcgacaaatacggcgtctctgacttttataagagactgatcaacaaagctaaaaccgtcgagggcgttgaggccctcaaacttcatattcttgcagctcttcctatgcatctcggtgataaacctgctcccaatactttctatgtcggtatttatattctgattgcagttggcgcagttatgatgtttgttggtttccttgggtgttacggtgcgatacaagagtcacagtgcctccttggtactttttttacatgtctggtaatattgttcgcttgtgaggttgcagcagggatctggggttttgtaaacaaggatcaaattgcaaaagatgtcaaacaattttacgaccaagccctgcaacaagcagtggtggacgacgatgctaataacgctaaggctgtagtaaaaacttttcatgaaactctggactgttgtggatcaacgcgtcagcacgacgaagctgtagatgccaactcactggcagaggccaaggtacttgccaaccgggagcttgataaatacggagtctctgatttttacaagagattgattaacaaggccaagaccgttgaaggtgtggaagccctcaaactgcacatccttgcggccttgcctacgcgttctacgcttacggcactcacaacttctgtattgaaaaataacttgtgtcccagcggtagcaacattataagcaaccttttcaaggaggactgccaccaaaaaattgacgacctgtttagcgggaagctctatctcatcgggatcgcggctattgtagtggcagtcataatgatatttgaaatgatactgtccatggttctgtgttgtggcattagaaacagttctgtttacggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 63 - DNA, 1380 bp, Artificial Sequence
  hCD81-ABD 1st-Nluc
atgggagtcgaaggatgcacgaagtgcatcaaatacctcttgtttgtttttaattttgtcttctggttggccggcggtgtaatccttggtgtagctctttggcttaggcatgatccgcaaactactaatttgctttatttggagatgcatcttgcagaagccaaggtccttgcgaatagagaactcgacaaatacggcgtctctgacttttataagagactgatcaacaaagctaaaaccgtcgagggcgttgaggccctcaaacttcatattcttgcagctcttcctatgcatctcggtgataaacctgctcccaatactttctatgtcggtatttatattctgattgcagttggcgcagttatgatgtttgttggtttccttgggtgttacggtgcgatacaagagtcacagtgcctccttggtactttttttacatgtctggtaatattgttcgcttgtgaggttgcagcagggatctggggttttgtaaacaaggatcaaattgcaaaagatgtcaaacaattttacgaccaagccctgcaacaagcagtggtggacgacgatgctaataacgctaaggctgtagtaaaaacttttcatgaaactctggactgttgtggatcaacgcgttctacgcttacggcactcacaacttctgtattgaaaaataacttgtgtcccagcggtagcaacattataagcaaccttttcaaggaggactgccaccaaaaaattgacgacctgtttagcgggaagctctatctcatcgggatcgcggctattgtagtggcagtcataatgatatttgaaatgatactgtccatggttctgtgttgtggcattagaaacagttctgtttacggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 64 - DNA, 1410 bp, Artificial Sequence
  hCD81-ABD 2nd-Nluc
atgggagtcgaaggatgcacgaagtgcatcaaatacctcttgtttgtttttaattttgtcttctggttggccggcggtgtaatccttggtgtagctctttggcttaggcatgatccgcaaactactaatttgctttatttggagatgcatctcggtgataaacctgctcccaatactttctatgtcggtatttatattctgattgcagttggcgcagttatgatgtttgttggtttccttgggtgttacggtgcgatacaagagtcacagtgcctccttggtactttttttacatgtctggtaatattgttcgcttgtgaggttgcagcagggatctggggttttgtaaacaaggatcaaattgcaaaagatgtcaaacaattttacgaccaagccctgcaacaagcagtggtggacgacgatgctaataacgctaaggctgtagtaaaaacttttcatgaaactctggactgttgtggatcaacgcgtcagcacgacgaagctgtagatgccaactcactggcagaggccaaggtacttgccaaccgggagcttgataaatacggagtctctgatttttacaagagattgattaacaaggccaagaccgttgaaggtgtggaagccctcaaactgcacatccttgcggccttgcctacgcgttctacgcttacggcactcacaacttctgtattgaaaaataacttgtgtcccagcggtagcaacattataagcaaccttttcaaggaggactgccaccaaaaaattgacgacctgtttagcgggaagctctatctcatcgggatcgcggctattgtagtggcagtcataatgatatttgaaatgatactgtccatggttctgtgttgtggcattagaaacagttctgtttacggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 65 - DNA, 1512 bp, Artificial Sequence
  Lamp2B-Nluc
atggtttgttttagactgttcccagtcccaggaagcgggttggttctggtatgtttggtacttggcgcagtgcgaagctacgcccttaagttcgaaaccggtggcgcgccaatgcatatttcacaagcagtacacgcagcccacgcagaaatcaacgaggcgggccgatctatcatcaactttgagaaattgtgtacatctgtgtatgacttttttgtgtggctgggcaggggacaccttctcgggcgccttgcggctattgtcgggaagcaggttcttctcggccggaaagttgtggtggtccgaagtcattgccattggaacgacctggcagtaataccagcgggggtggtacacaactgggacttcgagcccaggaaagttagtccatctaaacccagcttccaggaatttgtagactgggagaacgtttcacctgaactcaattctacagatcagccgtttctctgtacaggttcaggatcagggtcaggaagcggcagtcttaagcctgcaggtattctgattccaatcatagtcggagctggtcttagcggacttattattgtaatagttattgcctatgtaataggtagaagaaaaagctacgccggagcgcaaactcttggcggtggcggctcatacattccggaggctccacgagatggacaggcatacgtcagaaaagatggggagtgggtactgctctcaaccttccttagtccagctaatggaacaccaacaccgacgcccacgcctaccggacggtctctgtatcccagtcttgaggacttgaaagtggacaaagttatacaggctcaaacagcctttagcgcgaatccggcaaatccggccatactcagtgaggcgtccgcgccaataccccatgatggaaacctgtacccccgcctgtatcctgagctatcacagtatatgggattgagcctagagggtggccaactgggcacgcccacaccaactcccacaccgaccgggggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

SEQ ID NO: 66 - DNA, 1686 bp, Artificial Sequence
  Lamp2B-ABD-Nluc
atggtttgttttagactgttcccagtcccaggaagcgggttggttctggtatgtttggtacttggcgcagtgcgaagctacgcccttaagttcgaacagcatgatgaggctgtagatgctaacagcttggccgaagctaaagtattggctaatagggaacttgataagtatggggttagtgacttttataaacggcttattaataaggccaaaactgtcgaaggggtggaagcacttaagcttcacatacttgcagcccttcccttcgaaaccggtggcgcgccaatgcatatttcacaagcagtacacgcagcccacgcagaaatcaacgaggcgggccgatctatcatcaactttgagaaattgtgtacatctgtgtatgacttttttgtgtggctgggcaggggacaccttctcgggcgccttgcggctattgtcgggaagcaggttcttctcggccggaaagttgtggtggtccgaagtcattgccattggaacgacctggcagtaataccagcgggggtggtacacaactgggacttcgagcccaggaaagttagtccatctaaacccagcttccaggaatttgtagactgggagaacgtttcacctgaactcaattctacagatcagccgtttctctgtacaggttcaggatcagggtcaggaagcggcagtcttaagcctgcaggtattctgattccaatcatagtcggagctggtcttagcggacttattattgtaatagttattgcctatgtaataggtagaagaaaaagctacgccggagcgcaaactcttggcggtggcggctcatacattccggaggctccacgagatggacaggcatacgtcagaaaagatggggagtgggtactgctctcaaccttccttagtccagctaatggaacaccaacaccgacgcccacgcctaccggacggtctctgtatcccagtcttgaggacttgaaagtggacaaagttatacaggctcaaacagcctttagcgcgaatccggcaaatccggccatactcagtgaggcgtccgcgccaataccccatgatggaaacctgtacccccgcctgtatcctgagctatcacagtatatgggattgagcctagagggtggccaactgggcacgcccacaccaactcccacaccgaccgggggatccgtgtttaccctcgaagatttcgttggagattggcggcagactgcaggctacaatctggaccaagtccttgagcaagggggcgtgtcctctcttttccaaaaccttggtgtgtcagtgaccccaatacagcggatcgttctcagtggcgagaacgggctgaagatagatatccatgtcatcataccttacgagggcctgtctggagatcaaatggggcagatagagaaaatcttcaaggtcgtgtacccagtagacgaccaccatttcaaagtaatcctccactacgggacactggtcatcgacggcgtaacccctaatatgatcgattactttgggcgaccttatgaaggaattgcagtatttgatggtaaaaagataaccgtcacaggtaccctgtggaatggaaacaagataatagacgagagactgatcaaccctgacggaagcttgctgttcagggttaccattaacggcgtgacaggatggcggctttgtgaacggatacttgca

(19) United States
(12) Patent Application Publication
(10) Pub. No.: US 2023/0301926 A1
(43) Pub. Date: Sep. 28, 2023

(54) EXTRACELLULAR VESICLES WITH IMPROVED HALF-LIFE

(71) Applicant: Evox Therapeutics Ltd, Oxford Oxfordshire (GB)

(72) Inventors: Xiuming LIANG, Stockholm (SE); Samir EL ANDALOUSSI, Huddinge (SE)

(21) Appl. No.: 18/012,115

(22) PCT Filed: Jul. 5, 2021
(86) PCT No.: PCT/EP2021/068526

§ 371 Date: Dec. 21, 2022

(30) Foreign Application Priority Data
Jul. 3, 2020 (GB) 2010278.6

(51) Int. Cl. A61K 9/50; C07K 14/705; C07K 14/765; A61K 35/12

(52) U.S. Cl. CPC A61K 9/5068; A61K 35/12; C07K 14/70596; C07K 14/765; C07K 2317/94; C07K 2319/61

(57) ABSTRACT

The present invention pertains to extracellular vesicles (EVs), wherein the EVs comprise domains present on the surface of the EV, which modify the circulation time of such EVs. Such EVs display improved pharmacokinetics and are therefore useful as therapeutics. Such EVs may also be useful for affinity purification of the EV drug substance.

### TECHNICAL FIELD

The present invention relates to genetically engineered extracellular vesicles (EVs) with improved pharmacokinetic profiles, increased half-life and increased tumor accumulation in vivo and increased stability whilst in storage. The present invention also relates to uses of said EVs in therapy, methods of production of said EVs and purification of said EVs.

### BACKGROUND ART

EVs (such as exosomes) are typically nanometer-sized vesicles produced endogenously by most cell types and functioning as the body’s natural transport system for proteins, nucleic acids, peptides, lipids, and various other molecules between cells. EVs have a number of potential therapeutic uses and EVs are already being investigated as delivery vehicles for protein, nucleic acid and small molecule therapeutics. However, the promising potential clinical applications of EVs for treatment of diseases is currently impacted by the rapid clearance of EVs after especially intravenous administration. Due to the short half-life, the majority of untargeted EVs home to the liver and spleen, meaning that the therapeutic applications of untargeted EV therapeutics are predominantly focused on these target organs.

The short circulation time of EVs in vivo is one of the major limitations to exploiting their considerable therapeutic potential. Furthermore, EVs are known to be predominantly taken up by the liver and the spleen. This distinct biodistribution pattern means that targeting organs other than the hepatosplenic system requires large doses and/or the inclusion of specific targeting moieties to direct the EVs to other target organs, which is a problem that the present invention seeks to overcome. It is desirable to divert EVs away from uptake by the liver and the spleen so that this will increase delivery to other organs and thus improve biodistribution (especially of targeted EVs for instance to the brain).

There are a number of commonly employed techniques to increase the half-life of drugs and biologics. Typically, one of four general strategies for prolongation of half-life are used:

1) Noncovalent binding or genetic fusion of a pharmacologically active peptide or protein to a naturally long-half-life protein or protein domain e.g., Fc fusion, transferrin, or albumin fusion or fusion to an inert polypeptide, e.g., XTEN, a homo-amino acid polymer, a proline-alanine-serine polymer, or an elastin-like peptide.

2) Increasing the hydrodynamic radius by chemical conjugation of the pharmacologically active peptide or protein to repeat chemical moieties, e.g., to polyethylene glycol (PEG) (PEGylation) or hyaluronic acid.

3) Significantly increasing the negative charge of the pharmacologically active peptide or protein by polysialylation; or, alternatively, fusing a negatively charged, highly sialylated peptide (e.g., carboxy-terminal peptide [CTP; of chorionic gonadotropin (CG) β-chain]), known to extend the half-life of natural proteins such as human CG β-subunit, to the biological drug candidate.

4) Coating or conjugating the pharmacological agents (e.g. lipid or polymer nanoparticle or a therapeutic protein) with PEG of different lengths and structure, in order to decrease the electrostatic charge of a cargo molecule and shield it from immune systems cells, renal or liver clearance mechanisms.

All these current approaches, except for PEGylation, have only been used successfully clinically with either recombinant proteins or in some instances RNA therapeutics, but never with a large macromolecular assembly in the form of a nanoparticle, such as an EV, which is impacted by completely different biophysical and immunological considerations in vivo. In particular, EVs in addition to being much larger than a single protein or RNA therapeutic, also carry a very different charge, which makes the translation of existing methods of altering pharmacokinetics! pharmacodynamics into the EV context very unpredictable.

However, the existing methods described above have some significant drawbacks. The main concern with Fc fusion is the stability, aberrant glycosylation in linkers and fusion proteins, while the main concern for HSA fusion is that the biodistribution may be more limited to specific organs. Fusion of Fc proteins is also bulky and can lead to problems with interference of the fusion protein with the activity of the therapeutic.

PEGylation of EVs has been attempted in the past with little success. Firstly, it is a challenge to conjugate PEG to the EVs without disturbing the EV topology and phenotype. Secondly, PEG is an artificial substance which may cause toxic side effects when injected. It is furthermore immunogenic, which can also cause toxicity as well as clearance of the drug product from circulation.

To use sialylation in the context of EVs to increase their half-life would be very unlikely to work, since there are receptors that recognise sialylated moieties that drive uptake for certain EV subclasses. For example, B-cell exosomes are taken up by sialoadhesin in spleen. To increase the glycosylation pattern on EVs is furthermore not as easy as it is for more simple protein therapeutics, since the EV already comprises a membrane, similar to the plasma membrane, covering the EVs; hence they already have glycosylated proteins on their surface. Proteins commonly found on EVs are known to be heavily glycosylated and thereby negatively charged. There is therefore probably no benefit to increase the sialylation of the EV surface.

### SUMMARY OF INVENTION

It is hence an object of the present invention to overcome the above-identified problems associated with half-life and biodistribution of EVs.

In a first aspect the present invention relates to an EV modified to comprise at least one albumin binding domain (ABD) present on the surface of the EV. The ABD may form part of a fusion protein with an EV protein, optionally wherein the EV protein is a transmembrane EV protein or an EV protein associated with the outer surface of the EV membrane.

In a second aspect the present invention relates to a method for producing an EV according to the first aspect, comprising: (i) introducing into an EV-producing cell at least one polynucleotide construct encoding an ABD-EV protein fusion construct; and (ii) expressing said construct in the EV-producing cell, thereby generating an EV comprising ABD present on the surface of the EV.

In a third aspect the present invention relates to a pharmaceutical composition comprising at least one EV of the first aspect and a pharmaceutically acceptable excipient or carrier.

In a fourth aspect the present invention relates to an EV of the first aspect and/or a pharmaceutical composition of the third aspect, for use in medicine.

### BRIEF DESCRIPTION OF FIGURES

FIG. 1: Western blot showing good expression of fusion constructs in stable cells.

FIG. 2: Western blot showing good expression of fusion constructs in EVs.

FIG. 3: In vivo half-life extension showing the presence of ABD significantly increased the half-life of EVs in circulation and, by making them more stable, resulted in more than 14 times more EVs in the circulation compared to the control group.

FIG. 4: In vivo biodistribution of ABD EVs showing significant fold increases in EVs in target organs compared to control EVs.

FIG. 5: In vivo biodistribution of ABD EVs showing tumor accumulation of ABD EVs compared to control EVs.

FIG. 6: In vivo biodistribution of ABD EVs showing lymph node accumulation of ABD EVs compared to control EVs.

FIG. 7: ABD-EV Albumin In vitro Binding Assay showing that, compared to control EVs, ABD EVs are able to bind albumin.

FIG. 8: Flow Cytometry analysis of ABD EVs′ capacity to bind albumin compared to control EVs.

FIG. 9: Further ABD-EV albumin in vitro binding assays testing a range of additional constructs,

FIG. 10: Further in vivo half-life extension experiments assays testing a range of additional constructs.

FIG. 11: In vitro albumin binding data combined with in vivo plasma half-life extension data for EVs expressing ABD fused to a single pass transmembrane EV protein.

FIG. 12: In vivo biodistribution of ABD EVs showing lymph node and tumor accumulation of ABD EVs compared to control EVs.

FIG. 13: Graphs showing ABD EV half-life in plasma after delivery by different routes of administration.

FIG. 14: Graphs showing improved plasma half-life of ABD EVs derived from an alternative cell source.

FIG. 15: Gradient separation (Dot-Blot quantification) & wide-field microscopy images showing co-location of albumin and ABD expressing EVs.

FIG. 16: Graph showing in vivo ABD binding assay expressed as quantified Dot-Blot confirming that in vivo EVs expressing ABD bind albumin.

FIG. 17: Further gradient separation analysis by Dot-Blot showing ABD EVs bind albumin.

FIG. 18: Schematic outlining construct design for fusion proteins.

### DETAILED DESCRIPTION OF THE INVENTION

The present invention relates to EVs comprising at least one ABD present on the surface of the EVs. The present invention also relates to methods of making and purifying those EVs and their use in therapy. The EVs of the present invention have a number of distinct advantages due to the presence of ABD on their surface. Without wishing to be bound by theory, principally the ABD present on the surface of the EVs is able to extend the half-life of the EVs in circulation, presumably by mediating interaction between the EV and serum albumin. This extension of half-life can be applied broadly across any and all EVs. It applies to EVs loaded with any cargo and any targeting moiety, i.e., this invention is not specific to the cargo or targeting moiety; it is broadly applicable. Previous attempts to increase half-life of biologics has required specific tailoring to the biologic in question. The present invention is remarkable in that it is applicable not to the therapeutic cargo, but to the delivery vesicle, thus making it extremely adaptable to increase the half-life of any cargo molecule that is capable of being loaded into or onto the EV. Without wishing to be bound by theory, the increased half-life of ABD EVs can also result in altered biodistribution of the ABD EVs. This alteration of EV biodistribution, and therefore the pharmacokinetics of the drug cargo carried in or on the EV, is critically important for prolonging the circulation time of the EV and improving the opportunities for targeted delivery of therapeutic EVs. Albumin proteins are also capable of binding to the recycling receptor FcRN, which is involved in receptor mediated endocytosis. As such, uptake of the EV or exosome into target cells and/or tissue is enabled. Without wishing to be bound by theory, this results in greater therapeutic efficacy, due to higher payloads being delivered to the target organ. For instance, ABD EVs are ideally suited for targeting the brain. By exploiting a protein such as albumin, all of the aforementioned drawbacks of known methods to increase the half-life of EVs, such as PEGylation, can also be avoided.

For convenience and clarity, certain terms employed herein are collected and described below. Unless otherwise defined, all technical and scientific terms used herein have the same meaning as commonly understood by one of ordinary skill in the art to which this invention belongs. In the specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. “About” can be understood as within 10%, 9%, 8%, 7%, 6%, 5%, 4%, 3%, 2%, 1%, 0.5%, 0.1%, 0.05%, or 0.01 % of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present specification, including definitions, will control. In addition, the materials, methods, and examples are illustrative only and are not intended to be limiting. Other features and advantages of the disclosure will be apparent from the following detailed description and claims.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the ABD proteins and ABD fusion proteins/polypeptides described herein in connection with the EVs are to be understood to be disclosed, relevant and compatible with all other aspects, teachings and embodiments herein, for instance aspects and/or embodiments relating to the methods for producing or purifying the EVs or relating to the corresponding polynucleotide constructs described herein or the engineered EV-producing cells from which the EVs derive. Furthermore, certain embodiments described in connection with certain aspects, for instance the administration routes of the EVs comprising the therapeutic cargo molecule and optionally the fusion polypeptides, as described in relation to aspects pertaining to treating certain medical indications, may naturally also be relevant in connection with other aspects and/or embodiments, such as those pertaining to the pharmaceutical compositions comprising such EVs. Furthermore, all polypeptides and proteins identified herein can be freely combined in fusion proteins using conventional strategies for fusing polypeptides. As a non-limiting example, albumin binding domain protein described herein may be freely combined in any combination with one or more EV proteins, optionally combined with all other polypeptide domains, regions, sequences, peptides, groups herein, e.g. any multimerization domains, linker sequences, release domains, therapeutic cargo molecules, endosomal escape domains and/or targeting moieties. Moreover, any and all features (for instance any and all members of a Markush group) can be freely combined with any and all other features (for instance any and all members of any other Markush group), e.g. any ABD may be combined with any EV protein. Furthermore, when teachings herein refer to EVs in singular and/or to EVs as discrete natural nanoparticle-like vesicles, it should be understood that all such teachings are equally relevant for and applicable to a plurality of EVs and populations of EVs. As a general remark, albumin binding domains, the EV proteins, the EV-producing cell sources, the additional domains and moieties, the therapeutic cargo molecules, the targeting moieties and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences, as long as any given molecule retains the ability to carry out the desired technical effect associated therewith. As long as their biological properties are maintained, the polypeptide and/or polynucleotide sequences according to the present application may deviate with as much as 50% (calculated using, for instance, BLAST or ClustalW) as compared to the native sequence, although a sequence identity or similarity that is as high as possible is preferable (for instance 60%, 70%, 80%, or e.g. 90% or higher). Standard methods in the art may be used to determine sequence identity or homology. For example, PILEUP and BLAST algorithms can be used to calculate homology or line up sequences. The combination (fusion) of e.g. several polypeptides implies that certain segments of the respective polypeptides may be replaced and/or modified and/or that the sequences may be interrupted by insertion of other amino acid stretches, meaning that the deviation from the native sequence may be considerable as long as the key properties (e.g. ability to bind albumin and therefore extend half-life, ability to traffic a fusion construct to an EV, targeting capabilities, etc.) are conserved. Similar reasoning thus naturally applies to the polynucleotide sequences encoding for such polypeptides. Any SEQ ID NOs mentioned herein in connection with peptides, polypeptides and proteins shall only be seen as examples and for information only, and all peptides, polypeptides and proteins shall be given their ordinary meaning as the skilled person would understand them. Thus, as above-mentioned, the skilled person will also understand that the present invention encompasses not merely the specific SEQ ID NOs and/or accession numbers referred to herein, but also variants and derivatives thereof, for example, removal of His tag (HHHHHH) from SEQ ID NOs: 5-10. All proteins, polypeptides, peptides, nucleotides and polynucleotides mentioned herein are to be construed according to their conventional meaning as understood by a skilled person.

The term “at least one” as used herein can mean at least two, at least three, at least four, at least five, at least six, at least seven, at least eight, at least nine, at least ten or more.

As used herein, the terms, “half-life” and “T½”, can mean the time it takes for the blood plasma concentration of a substance to halve its steady-state (plasma half-life) when circulating in the full blood of an organism.

The terms “extracellular vesicle” or “EV” or “exosome” or “genetically modified/genetically engineered EV/exosome”, or “engineered/modified EV/exosome” are used interchangeably herein and can be understood to relate to any type of vesicle that is obtainable from a cell in any form, for instance a microvesicle (e.g. any vesicle shed from the plasma membrane of a cell), an exosome (e.g. any vesicle derived from the endosomal, lysosomal and/or endo-lysosomal pathway and/or from the plasma membrane or any other membrane of a cell), an apoptotic body (obtainable from apoptotic cells, ARRDC1-mediated microvesicles (ARMMs) (arrestin domain containing protein 1 [ARRDC1 ]-mediated microvesicles), a microparticle (which may be derived from platelets), an ectosome (derivable from e.g. neutrophils and monocytes in serum), prostatosome (e.g. obtainable from prostate cancer cells), or a cardiosome (e.g. derivable from cardiac cells), etc. Exosomes, microvesicles and ARMMs represent particularly preferable EVs, but other EVs may also be advantageous in various circumstances. The terms “genetically modified” and “genetically engineered” EV indicates that the EV is derived from a genetically modified/engineered cell usually comprising a recombinant fusion protein product which is incorporated into the EVs produced by those cells. The term “modified EV” indicates that the vesicle has been modified either using genetic or chemical approaches, for instance via genetic engineering of the EV-producing cell or via e.g. chemical conjugation, for instance to attach moieties to the exosome surface.

The size of EVs may vary considerably, but an EV typically has a nano-sized hydrodynamic radius, i.e. a radius below 1000 nm. Exosomes often have a size of between 30 and 300 nm, typically in the range between 40 and 250 nm, which is a highly suitable size range. Clearly, EVs may be derived from any cell type, both in vivo, ex vivo and in vitro (further details of source cells are described below).

The term “albumin binding domain” or “ABD” can be understood to relate to any protein, peptide, antibody or nanobody, or fragment or domain thereof capable of binding to albumin. ABDs may be derived from any species; preferably the ABD has specific binding affinity for human serum albumin (HSA). Commonly known ABDs are antibodies or nanobodies that are raised against albumin or ABDs derived from PAB protein from Peptostreptococcus magnus and protein G from group C and G streptococci, both of which bind to albumin with high affinity.

ABDs are small, three-helical protein domains found in various surface proteins often expressed, for instance, by gram-positive bacteria. ABDs may be engineered by specific mutagenesis to achieve a broader specificity for different albumin, an increased stability, lower immunogenicity or an improved binding affinity. The albumin bound by the ABD in the present invention may be recombinant albumin or albumin derived from any species, but preferably it is HSA.

Exemplary ABD sequences used in the present invention include: SEQ ID NO: 1 (G418-GA3, ABD001 (WT)) SEQ ID NO:2 (ABD011), SEQ ID NO:3 (ABD013), and SEQ ID NO:4 (ABD035). The present invention also encompasses variants or derivatives of these sequences, which have at least a 50%, 60%, 70%, 75%, 80%, 85%, 90%, 95% or 99% sequence identity or homology to these sequences and are capable of binding to albumin. The present invention relates to EVs comprising ABDs present on the surface of the EVs, wherein the ABD has about 50%, 60%, 70%, 80%, 90% or 95% sequence identity or homology to any of SEQ ID NOs:1-4. The ABDs may be synthesized using standard peptide synthesis methods known in the art.

The ABD of the present invention may be an antibody, single-chain variable fragment (scFv) nanobody, heavy chain antibody (hcAb), single domain antibody (sdAb) such as VHH or VNAR, or a fragment thereof, which is capable of binding to albumin. sdAbs and antibody fragments are particularly preferred due to their small size, which allows for other additional domains to be introduced into the fusion protein and simple construct generation and expression.

The ABDs according to the present invention are present on the surface of the EVs so that they are able to bind to albumin found primarily in the circulatory systems of patients to be treated or albumin present in the drug product formulation or in any storage buffer or other solution to which the EV is exposed prior to administration to a patient. The ABD may be presented on the surface of the EV in any number of ways known to the skilled person, provided that the ABD is exposed on the outer surface of the EV such that it is capable of binding albumin. Commonly the ABD will form part of a fusion protein with an EV protein. The EV may comprise more than one ABD, i.e. a plurality of ABDs. The plurality of ABDs may be the same or different from one another.

The present invention does not require the inclusion of the full albumin protein in the fusion construct (as has often been the approach in the past for increasing half-life of biologics). Instead the present invention relies on the binding of the EV to the subject’s native albumin after injection into circulation or binding ex vivo to albumin present in e.g. a formulation or a buffer. Albumin has a Mr of 66.5 kDa; ABDs are typically much smaller than albumin itself. Without wishing to be bound by theory, the smaller size of the ABD allows for simpler generation of stable cell lines, enables more copies of ABD to be fused into the fusion polypeptides of the invention and is also beneficial because the small size is better for allowing other proteins, such as cargo proteins and/or targeting moieties, to be included within the same fusion construct. Without wishing to be bound by theory, this can allow the ABD EVs to act as a highly flexible platform from which any number of different therapeutic EVs can be produced because the ABD EVs are able to easily be modified to accommodate more cargo/s and/or targeting moieties.

In one embodiment, EVs of the present invention comprise at least one ABD present on the surface of the EV, wherein the ABD forms part of a fusion protein with an EV protein.

In another embodiment, EVs of the present invention comprise at least one ABD present on the surface of the EV, wherein the ABD forms part of a fusion protein with a transmembrane EV protein or an EV protein associated with the outer surface of the EV membrane. Inclusion of an EV protein as part of a fusion with ABD enables the ABD to be actively loaded into the EVs because the presence of the EV protein actively drives loading of the fusion construct into the EV. The EV protein used in this fusion protein may be a single or multi-pass transmembrane protein.

The EV protein which is comprised in a fusion protein as per the present invention may be selected from the group consisting of the following non-limiting examples: CD9, CD53, CD63, CD81, CD54, CD50, FLOT1, FLOT2, CD49d, CD71, CD133, CD138, CD235a, AAAT, AT1B3, AT2B4, ALIX, Annexin, BASI, BASP1, BSG, Syntenin-1, Syntenin-2, Lamp2, Lamp2a, Lamp2b, TSN1, TSN3, TSN4, TSN5 TSN6, TSN7, TSPAN8, TSN31, TSN10, TSN11, TSN12, TSN13, TSN14, TSN15, TSN16, TSN17, TSN18, TSN19, TSN2, TSN4, TSN9, TSN32, TSN33, TNFR, TfR1, syndecan-1, syndecan-2, syndecan-3, syndecan-4,, CD37, CD82, CD151, CD224, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, CLIC1, CLIC4, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD53, CD86, CD110, CD111, CD115, CD117, CD125, CD135, CD184, CD200, CD279, CD273, CD 274, CD362, COL6A1, AGRN, EGFR, FPRP, GAPDH, GLUR2, GLUR3, GP130, GPI anchor proteins, GTR1, HLAA, HLA-DM, HSPG2, ITA3, Lactadherin, L1CAM, LAMB1, LAMC1, LIMP2, MYOF, ARRDC1, ATP2B2, ATP2B3, ATP2B4, BSG, IGSF2, IGSF3, IGSF8, ITGB1, ITGA4, ATP1A2, ATP1A3, ATP1A4, ITGA4, SLC3A2, ATP transporters, ATP1A1, ATP1B3, ATP2B1, LFA-1, LGALS3BP, Mac-1 alpha, Mac-1 beta, MFGE8, a member of the myristoylated alanine rich Protein Kinase C substrate (MARCKS) protein family such as MARCKSL1, matrix metalloproteinase-14 (MMP14), PDGFR, PTGFRN, PRPH2, ROM1, SLIT2, SLC3A2, SSEA4, STX3, TCRA, TCRB, TCRD, TCRG, TFR1, UPK1A, UPK1B, VTI1A, VTI1B, and any other EV proteins, and any combinations, derivatives, domains, variants, mutants, or regions thereof. Mutations may be introduced into the wild type sequence of the EV protein to alter its function. A preferred mutant according to the invention is CD63(Y235A). Without wishing to be bound by theory, the use of EV proteins has the effect of driving loading of the ABD into EVs, such that ABD is actively loaded into EVs (as above). As a result, EV proteins are sometimes referred to as carrier proteins. Particularly advantageous EV proteins include tetraspanins, CD63, CD81, CD9, CD82, CD44, CD47, CD55, LAMP2B, LIMP2, ICAMs, integrins, ARRDC1, syndecan, syntenin, TNFR, TfR1, and Alix, as well as derivatives, domains, variants, mutants, or regions thereof.

In another embodiment, the EVs of the present invention comprise at least one ABD present on their surface, wherein the ABD is engineered into an extravesicular loop or loops of a multi-pass transmembrane protein, optionally wherein the multi-pass transmembrane protein is a tetraspanin such as CD36, CD9, CD81 or any of TSPAN1 -TSPAN33. The ABD may be fused into the first, second, third, fourth or any subsequent loop of the multi-pass transmembrane EV protein or into more than one of the loops. Suitably the ABD is fused into the first, second or any subsequent loop of the multi-pass transmembrane EV protein. The present inventors have surprisingly found that incorporation of the ABD into the loops of transmembrane proteins does not prevent the expression of the fusion protein nor its insertion into the membrane and importantly it has been shown that ABD in the loop(s) of the transmembrane protein also does not affect the structure and function of the ABD despite this highly a-typical fusion protein location. Advantageously, ABDs may be incorporated into more than one of the loops of said multi-pass transmembrane protein and/or more than one ABD i.e. a plurality of ABDs, may be incorporated into each loop of said multi-pass transmembrane protein. The plurality of ABDs present in the loop or loops may be the same or different ABDs. The data in the present application show that more than one ABD may be incorporated into the transmembrane fusion protein without affecting the expression of the transmembrane protein. The presence of more than one ABD per transmembrane EV protein means that more albumin molecules can be bound to the EV per fusion protein expressed. This is advantageous as it increases the amount of albumin coating the EV and thus increases the shielding effect of the ABD. This, in turn, increases the half-life of the ABD EV, whilst allowing the additional constructs to be expressed in the same EV, for instance, additional constructs may comprise therapeutic cargos and/or targeting moieties. The engineering of ABD into one loop of a multi-pass transmembrane EV protein and another entity, such as a targeting entity or therapeutic cargo protein into another loop of the same transmembrane EV protein allows multiplexing of different components into a single fusion protein which dramatically increases the versatility of the resultant EV. Multiplexing of features enables this engineered EV technology to become a highly versatile platform technology where component parts can be added or exchanged with relative ease. Technically, having more than one feature on a single EV protein is also simpler as it only requires generation of a single stable cell line but creates an EV with multiple different functional elements.

In another embodiment, the ABD may be fused to the N terminal domain (NTD) or C terminal domain (CTD) of the EV protein.

In another embodiment, the EVs of the present invention comprise additional sequences or domains within the ABD EV protein fusion protein construct. In one advantageous embodiment, the ABD EV protein further comprises at least one multimerization domain.

Multimerization domains according to the present invention may be homomultimerization domains or heteromultimerization domains. The multimerization domains of the present invention may be dimerization domains, trimerization domains, tetramerization domains, or any higher order of multimerization domain. Without wishing to be bound by theory, multimerization domains can enable dimerization, trimerization, or any higher order of multimerization of the fusion polypeptides, which increases the sorting and trafficking of the fusion polypeptides into EVs and may also contribute to increasing the yield of vesicles produced by EV-producing cells. Exemplary multimerization domains include, but are not limited to: leucine zipper, fold-on domain, fragment X, collagen domain, 2G12 IgG homodimer, mitochondrial antiviral-signaling protein CARD filament, Cardiac phospholamban transmembrane pentamer, parathyroid hormone dimerization domain, Glycophorin A transmembrane, human immunodeficiency virus (HIV) Gp41 trimerisation domain, HPV45 oncoprotein E7 C-terminal dimer domain, and any combination thereof.

In another advantageous embodiment, the ABD EV protein fusion protein further comprises at least one endosomal escape domain. Endosomal escape domains according to the present invention include: HA2, VSVG, GALA, B18. Other exemplary endosomal escape domains include, but are not limited to: HIV TAT PDT (peptide/protein transduction domain), HIV Gp-120, KALA, GALA and INF-7 (derived from the N-terminal domain of influenza virus hemagglutinin HA-2 subunit), endosomal escape moieties that act by causing membrane fusion such as Diphtheria toxin T domain, proton sponge type endosomal escape moieties such as peptides or lipids with histidine or imidazole moieties and cell penetrating peptides (CPPs) and other moieties that enable endosomal escape. As would be appreciated by the skilled person, CPPs are typically less than 50 amino acids, but may also be longer, are typically highly cationic and rich in arginine and/or lysine amino acids and have the ability to gain access to the interior of virtually any cell type. Exemplary CPPs are transportan, transportan 10, penetratin, MTS, VP22, CADY peptides, MAP, KALA, PpTG20, proline-rich peptides, MPG peptides, PepFect peptides, Pep-1, L-oligomers, calcitonin peptides, various arginine-rich CPPs, such as poly-Arg, tat and combinations thereof. The presence of endosomal escape domains advantageously assists to drive endosomal escape and thereby enhance the bioactive delivery of the EV per se. Use of endosomal escape strategies can be used in the treatment of diseases where the cargo carried within the EV is required to be delivered into the cytosol of the recipient cell or within any other compartment that is outside of the endo-lysosomal system.

In another advantageous embodiment, the ABD EV protein fusion protein further comprises at least one linker, spacer and/or scaffold sequence. The presence of linkers, spacers and/or scaffold sequences allows flexibility and enables ABD to be positioned optimally for display on the surface of the EV.

Linkers according to the invention are useful in providing increased flexibility, improving pharmacokinetics (PK), increasing expression and improving biological activity of the fusion polypeptide constructs, and also to the corresponding polynucleotide constructs, and may also be used to ensure avoidance of steric hindrance and maintained functionality of the fusion polypeptides. Exemplary linkers according to the present invention include glycine or serine linkers, which increase stability or flexibility, such as (GGGGS)ₙ (n=1, 2, 4) or (Gly)₆, (Gly)₈, rigid linkers such as (EAAAK)n (n=1-3, and A(EAAAK)4ALEA(EAAAK)4A, bending linkers (XP)ₙ or cleavable linkers such as disulphide, protease sensitive sequences.

In a specific embodiment, an EV of the invention comprises more than one ABD, i.e. a plurality of ABDs. The plurality of ABDs may be the same or different from one another. As described above this may be as a result of more than one ABD being present in a single fusion protein, alternatively it may be as a result of multiple fusion proteins being loaded into a single EV. Said multiple fusion proteins may comprise the same or different EV protein. Without wishing to be bound by theory, the presence of more than one ABD on a single EV can be advantageous as it increases the amount of albumin coating the EV and thus increases the shielding effect of the ABD, which in turn can increase the half-life of the ABD EV. In some embodiments, the EVs of the invention comprises at least one ABD EV fusion protein, wherein the at least one ABD EV fusion protein comprises at least one, or at least two, or at least three, or at least four, or at least five, or at least six, or at least seven, or at least eight, or at least nine, or at least ten ABDs.

EVs comprising ABDs may be produced using any of the methods disclosed herein.

In one embodiment of the present invention, the EVs of the invention are further loaded with a therapeutic cargo, optionally wherein the therapeutic cargo is a protein, nucleic acid, virus, viral genome, antigen or small molecule or combination thereof.

The cargo to be loaded according to the present invention may be essentially any type of drug cargo, such as for instance: a nucleic acid such as an RNA molecule, a DNA molecule or a mixmer, mRNA, an antisense or splice-switching oligonucleotide, siRNA, shRNA, miRNA, plasmid DNA (pDNA), a supercoiled or unsupercoiled plasmid, or a mini-circle; a peptide or protein including: a transporter, enzyme, receptor such as a decoy receptor, membrane protein, cytokine, antigen or neoantigen, ribonuclear protein, nucleic acid binding protein, antibody, nanobody or an antibody fragment; an antibody-drug conjugate; a small molecule drug; gene editing technology such as CRISPR-Cas9, a TALEN, meganuclease; or a vesicle-based cargo such as a virus (e.g. an AAV, lentivirus, etc.). In one embodiment, the cargo may be a mixture of protein, nucleic acid, virus, viral genome, antigen and/or small molecule.

In more detail, the nucleic acid cargo molecule of the invention may be selected from the group comprising shRNA, siRNA, saRNA, miRNA, an anti-miRNA, mRNA, gRNA, pri-miRNA, pre-miRNA, circular RNA, piRNA, tRNA, rRNA, snRNA, IncRNA, ribozymes, mini-circle DNA, plasmid DNA, RNA/DNA vectors, trans-splicing oligonucleotides, splice-switching oligonucleotides, CRISPR guide strands, morpholinos (PMO) antisense oligonucleotides (ASO), peptide-nucleic acids (PNA), a viral genome and viral genetic material (for instance, a naked AAV genome), but essentially any type of nucleic acid molecule can be delivered by the EVs of the present invention. Both single-stranded and double-stranded nucleic acid molecules are within the scope of the present invention, and the nucleic acid molecule may be naturally occurring (such as RNA or DNA) or may be a chemically synthesised RNA and/or DNA molecule, which may comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, thionucleotides, phosphoramidate, PNA, PMO, etc.

A “nucleic acid” refers to a polynucleotide and includes polyribonucleotides and poly-deoxyribonucleotides. Nucleic acids according to the present invention may include any polymer or oligomer of pyrimidine and purine bases, e.g., cytosine (C), thymine (T) and uracil (U), and adenine (A) and guanine (G), respectively (see Albert L. Lehninger, Principles of Biochemistry, at 793-800 (Worth Pub. 1982) and G. Michael Blackburn, Michael J. Gait, David Loakes and David M. Williams, Nucleic Acids in Chemistry and Biology 3^{rd} edition, (RSC publishing 2006), which are herein incorporated in their entirety for all purposes). Indeed, the present invention contemplates any deoxyribonucleotide or ribonucleotide component, and any chemical variants thereof. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” can mean a nucleic acid ranging from at least 2, at least 8, at least 15 or at least 25 nucleotides in length, but may be up to 50, 100, 1000, 5000, 10000, 15000, or 20000 nucleotides long or a compound that specifically hybridises to a polynucleotide. Polynucleotides include sequences of DNA or RNA or mimetics thereof, which may be isolated from natural sources, recombinantly produced or artificially synthesised. A further example of a polynucleotide as employed in the present invention may be a peptide nucleic acid (PNA; see U.S. Patent No. 6,156,501, which is hereby incorporated by reference in its entirety.) The invention also encompasses situations in which there is a non-traditional base pairing, such as Hoogsteen base pairing, which has been identified in certain tRNA molecules and postulated to exist in a triple helix. “Polynucleotide” and “oligonucleotide” are used interchangeably herein. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, “polynucleotide” includes, for instance, cDNA, RNA, DNA/RNA hybrid, antisense RNA, siRNA, mRNA, ribozyme, genomic DNA, synthetic forms, and mixed polymers, both sense and antisense strands, and may be chemically or biochemically modified to contain non-natural or derivatised, synthetic, or semi-synthetic nucleotide bases. Also contemplated are alterations of a wild type or synthetic gene, including, but not limited to, deletion, insertion, substitution of one or more nucleotides, or fusion to other polynucleotide sequences.

The present invention specifically relates to ABD EVs which are further loaded with nucleic acids such as siRNAs, which target oncogenes known to be involved with the development of cancer. The genes targeted by the nucleic acids according to the present invention may be ABL, AF4/HRX, AKT-2, ALK, ALK/NPM, AML1, AML1/MTG8, AXL, BCL-2, 3, 6, BCR/ABL, c-MYC, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HER2/neu, HOX11, HST, IL-3, INT-2, JUN, KIT, KS3, K-SAM, LBC, LCK, LMO1, LMO2, L-MYC, LYL-1, LYT-10, LYT-10/Ca1, MAS, MDM-2, MLL, MOS, MTG8/AML1, MYB, MYH11/CBFB, NEU, N-MYC, OST, PAX-5, PBX1/E2A, PIM-1, PRAD-1, RAF, RAR/PML, RAS-H, RAS-K, RAS-N, REL/NRG, RET, RHOM1, RHOM2, ROS, SKI, SIS, SET/CAN, SRC, TAL1, TAL2, TAN-1, TIAM1, TSC2, TRK.

In more detail, the therapeutic protein cargos according to the present invention include: antibodies, intrabodies, nanobodies, scFvs, affibodies, bi- and multi-specific antibodies or binders including bispecific T-cell engagers (BiTEs), receptors, ligands, transporters, enzymes for e.g. enzyme replacement therapy (ERT) or gene editing, tumour suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance, pseudomonas exotoxins), structural proteins, neurotrophic factors such as NT¾, brain-derived neurotrophic factor (BDNF) and nerve growth factor (NGF) and its individual subunits such as the 2.5S beta subunit, ion channels, membrane transporters, proteostasis factors, proteins involved in cellular signaling, translation-and transcription-related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins such as cytokines and inhibitors of such cytokines (cytokines may include: CXCL8, GMCSF, interleukins including: IL-1 family, IL-2, IL-4, IL-6, IL-6-like, IL-9, IL-10, IL12, IL-13, IL-17, interferons including INF-alpha/beta/gamma, TNF family members, CD40 and CD40L, TRAIL, and TGF-beta family), mitochondrial proteins, and heat shock proteins, etc. The cargo protein may be a reporter protein such as green fluorescent protein (GFP) or nanoLuc. In one preferred embodiment, the encoded protein is a CRISPR-associated (Cas) polypeptide (such as Cas9) with intact nuclease activity, which is associated with (i.e. carries with it) an RNA strand that enables the Cas polypeptide to carry out its nuclease activity in a target cell once delivered by the peptide. Alternatively, in another preferred embodiment, the Cas polypeptide may be catalytically inactive, to enable targeted genetic engineering. Yet another alternative may be any other type of CRISPR effector such as the single RNA guided endonuclease, Cpf1. The inclusion of Cpf1 is a particularly preferred embodiment of the present invention, as it cleaves target DNA via a staggered double-stranded break. Cpf1 may be obtained from species such as Acidaminococcus or Lachnospiraceae. In yet another exemplary embodiment, the Cas polypeptide may also be fused to a transcriptional activator (such as the P3330 core protein), to specifically induce gene expression.

The present invention also relates, in some embodiments, to nucleic acid cargos, which are loaded by fusion of an EV protein to a nucleic acid binding protein (NA-binding protein), which then binds to the nucleic acid cargo molecule and causes loading of the nucleic acid cargo into the EV. Non-limiting examples of NA-binding proteins are hnRNPA1, hnRNPA2B1, DDX4, ADAD1, DAZL, ELAVL4, IGF2BP3, SAMD4A, TDP43, FUS, FMR1, FXR1, FXR2, EIF4A13, the MS2 coat protein, as well as any domains, parts or derivates, thereof. More broadly, particular subclasses of RNA-binding proteins and domains, e.g. mRNA binding proteins (mRBPs), pre-rRNA-binding proteins, tRNA-binding proteins, small nuclear or nucleolar RNA-binding proteins, non-coding RNA-binding proteins, miRNA-binding proteins, shRNA-binding proteins and transcription factors (TFs). Furthermore, various domains and derivatives may also be used as an NA-binding domain to transport a nucleic acid cargo into EVs. Non-limiting examples of RNA-binding domains include small RNA-binding domains (RBDs) (which can be both single-stranded and double-stranded RBDs (ssRBDs and dsRBDs) such as DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1 CTR, PAM2, Xpo1, Piwi, CSD, and Ribosomal_L7Ae). Such RNA-binding domains may be present in a plurality, alone or in combination with others, and may also form part of a larger RNA-binding protein construct as such, as long as their key function (i.e. the ability to transport a nucleic acid cargo of interest, e.g. an mRNA or a short RNA) is maintained.

In preferred embodiments, the present invention relates to two groups of NA-binding domains, namely PUF proteins and CRISPR-associated polypeptides (Cas), specifically Cas6 and Cas13, as well as various types of NA-binding aptamers.

The present invention uses the term “PUF proteins” to encompass all related proteins and domains of such proteins (which may also be termed “PUM proteins”), for instance, human Pumilio homolog 1 (PUM1), PUMx2 or PUFx2, which are duplicates of PUM1, etc., or any NA-binding domains obtainable from any PUF (PUM) proteins. As would be appreciated by the skilled person, PUF proteins are typically characterized by the presence of eight consecutive PUF repeats, each of approximately 40 amino acids, often flanked by two related sequences, Csp1 and Csp2. Each repeat has a core consensus’ containing aromatic and basic residues. The entire cluster of PUF repeats is required for RNA binding. Remarkably, this same region also interacts with protein co-regulators, and is sufficient to rescue, to a large extent, the defects of a PUF protein mutant, which makes the PUF proteins highly suitable for mutations used in the present invention. Furthermore, PUF proteins are examples of releasable NA-binding domains which bind with suitable affinity to nucleic acid cargo molecules, thereby enabling a releasable, reversible attachment of the PUF protein to the nucleic acid cargo. PUF proteins are found in most eukaryotes and are involved in embryogenesis and development. PUFs have one domain that binds RNA that is composed of 8 repeats generally containing 36 amino acids, which is the domain typically utilized for RNA binding in the present invention. Each repeat binds a specific nucleotide and it is commonly the amino acids in position 12 and 16 that confer the specificity with a stacking interaction from amino acid 13. The PUFs can bind the nucleotides, adenosine, uracil and guanosine, and engineered PUFs can also bind the nucleotide, cytosine. Hence the system is modular and the 8-nucleotide sequence that the PUF domain binds to can be changed by switching the binding specificity of the repeat domains. Hence, the PUF proteins as per the present invention can be natural or engineered to bind anywhere in an RNA molecule, or alternatively one can choose PUF proteins with different binding affinities for different sequences and engineer the RNA molecule to contain said sequence. There is furthermore engineered and/or duplicated PUF domains that bind 16 nucleotides in a sequence-specific manner, which can also be utilized to increase the specificity for the nucleic acid cargo molecule further. Hence the PUF domain can be modified to bind any sequence, with different affinity and sequence length, which makes the system highly modular and adaptable for any RNA cargo molecule as per the present invention. PUF proteins and regions and derivatives thereof that may be used as NA-binding domains as per the present invention include the following non-limiting list of PUF proteins: FBF, FBF/PUF-8/PUF-6,-7,-10, all from C. elegans; Pumilio from D. melanogaster, Puf5p/Mpt5p/Uth4p, Puf4p/Ygl014wp/Ygl023p, Puf5p/Mpt5p/Uth4p, Puf5p/Mpt5p/Uth4p, Puf3p, all from S. cerevisiae; PufA from Dictyostelium; human PUM1 (Pumilio 1, sometimes known also as PUF-8R) and any domains thereof, polypeptides comprising NA-binding domains from at least two PUM1, any truncated or modified or engineered PUF proteins, such as for instance PUF-6R, PUF-9R, PUF-10R, PUF-12R, and PUF-16R or derivatives thereof; and X-Puf1 from Xenopus. Particularly suitable NA-binding PUFs as per the present invention include the following: PUF 531, PUF mRNA loc (sometimes termed PUFengineered or PUFeng), and/or PUFx2, (sequences of which are available in International Patent Publication No. WO 2019/092145) and any derivatives, domains, and/or regions thereof. The PUF/PUM proteins are highly advantageous as they may be selected to be of human origin.

Furthermore, as is the case with the PUF proteins, Cas proteins such as Cas6 and Cas13 are examples of releasable NA-binding domains, which bind with suitable affinity to nucleic acid cargo molecules, thereby enabling a releasable, reversible attachment of the Cas protein to the nucleic acid cargo. As with the PUF-based NA-binding domains, the Cas proteins represent a releasable, irreversible NA-binding domain with programmable, modifiable sequence specificity for the target nucleic acid cargo molecule, enabling higher specificity at a lower total affinity, thereby allowing for both loading of the nucleic acid cargo into EVs and release of the nucleic acid cargo in a target location.

Additional preferred embodiments include therapeutic protein cargos selected from the group comprising enzymes or transporters for lysosomal storage disorders, for instance, glucocerebrosidases such as imiglucerase, alpha-galactosidase, alpha-L-iduronidase, iduronate-2-sulfatase and idursulfase, arylsulfatase, galsulfase, acid-alpha glucosidase (GAA), sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alphaN-acetyl neuraminidase, GlcNAc phosphotransferase, mucolipin1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2 and hexoaminidase.

Additional preferred embodiments include therapeutic protein cargos selected from the group comprising enzymes associated with urea cycle disorders including: N-acetylglutamate synthase, carbamoyl phosphate synthetase, ornithine transcarbamoylase (OTC), argininosuccinic acid synthase, argininosuccinic acid lyase, arginase, mitochondrial ornithine transporter, citrin, y+L amino acid transporter 1 and uridine monophosphate synthase (UMPS).

In other preferred embodiments, the therapeutic protein cargo may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, mini-dystrophin, micro-dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The therapeutic protein cargos are typically proteins or peptides of human origin unless indicated otherwise by their name, any other nomenclature, or as known to a person skilled in the art, and they can be found in various publicly available databases such as Uniprot, RCSB, etc.

In another preferred embodiment, the therapeutic cargo is an antigen/neoantigen, optionally wherein the antigen/neoantigen is suitable for use in cancer immunotherapy.

Any antigen/neoantigen may be incorporated into the EVs of the present invention. The antigens may be suitable for raising immune responses against pathogens, such as bacteria, viruses and funguses, or the antigen may be a tumor antigen useful in eliciting an immune response against a tumor for cancer immunotherapy. There may be one or more antigens/neoantigens present in any EV according to the invention. The one or more antigens/neoantigens may be endogenous/autologous (coming from the subject itself) or exogenous/ allogenic (coming from another subject) or, in the case of more antigens/neoantigens being incorporated into/onto the EVs, the antigens/neoantigens may be any mix of autologous /allogenic antigens. Preferably the antigens are autologous. Moreover, the one or more antigens/neoantigens may have any origin, such as e.g. viral or bacterial, or may be a tumour antigen and, furthermore, may be immunostimulatory or immunosuppressive or a combination thereof. The antigen/neoantigen maybe be useful in the treatment of any disease by immunotherapy. The treatment of cancer by immunotherapy is a particularly preferred embodiment. Where the antigen is a neo-antigen it may be identified by sequencing of a tumour to identify the neo-antigen.

Exemplary tumour antigens include, but are not limited to: alphafetoprotein (AFP), carcinoembryonic antigen (CEA), CA-125, MUC-1, epithelial tumor antigen (ETA), melanoma-associated antigen (MAGE), WT-1, NY-ESO-1, LY6K, IMP3, DEPDC1, CDCA-1,abnormal products of ras, p53, KRAS, or NRAS, CTAG1B, peptides derived from chromosomal translocations such as BCR-ABL or ETV6-AML1, viral antigens such as peptides from HPV-related cancers, peptides derived from proteins such as tyrosinase, gp100/pmel17, Melan-A/MART-1, gp75/TRP1, or TRP2, and overexpressed antigens such as MOK (RAGE-1), ERBB2 (HER2/NEU).

Where the therapeutic cargo is an antigen or neoantigen, the EV or pharmaceutical composition comprising the EV (discussed further below) may optionally further comprise at least one adjuvant. Where the antigen is administered with an adjuvant to stimulate the immune response, the adjuvant may be, but is not limited to: an inorganic compound, such as aluminium hydroxide, aluminium phosphate, calcium phosphate hydroxide, a mineral oil such as paraffin oil, bacterial products such as killed bacteria Bordetella pertussis, Mycobacterium bovis, toxoids, a nonbacterial organic such as squalene, a detergent such as Quil A, a plant saponin, a cytokine such as IL-1, IL-2, IL-12, or Ribi Adjuvants (muramyl dipeptides) or immunostimulating complexes (ISCOM) such as stimulator of interferon genes (STING) agonists, which can include cyclic dinucleotides. Such adjuvants may protect the therapeutic EV from rapid dispersal by sequestering it in a local deposit, or they may contain substances that stimulate the host to secrete factors that are chemotactic for macrophages and other components of the immune system. Adjuvants that can be incorporated into a vaccine are well-known by a person skilled in the art and will be selected in such a way that they do not negatively affect the immunological activity of the EV.

The present invention also relates to ABD EVs which are loaded with viral cargos, optionally wherein the ABD EV further comprises one or more immune effector molecules that provide immune effector functions. Exemplary viral cargos include, but are not limited to, a viral vector, which is an adeno-associated viral (AAV) vector or a lentiviral vector. In some embodiments, the viral vector is an AAV vector. In some embodiments, the AAV vector comprises a capsid from human AAV serotype AAV1, AAV2, AAV3, AAV4, AAV5, AAV6, AAV7, AAV8, AAV9, AAV 10, AAV11 or AAV12. In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9 or AAV10. In some embodiments, the AAV capsid and the AAV ITR are from the same serotype or from different serotypes.

In some embodiments of the above aspects, the viral vector is a lentiviral vector. In some embodiments, the lentiviral vector is derived from HIV, a simian immunodeficiency virus or a feline immunodeficiency virus. In some embodiments, the lentiviral vector is non-replicating. In some embodiments, the lentiviral vector is non-integrating.

In some embodiments, the viral vector comprises a viral capsid and a viral genome, the viral genome comprising one or more heterologous transgenes. In preferred embodiments, the heterologous transgene encodes a polypeptide or protein. The protein encoded within the viral genome may be any one of the protein cargos according to the invention allowing the viral cargo to act as a gene replacement therapy.

In some embodiments, the cargo-loaded ABD EV may additionally comprise one or more molecules that provide immune effector functions. Immune effector molecules are particularly useful in the case of ABD EVs loaded with a viral (e.g. AW or lentiviral) cargo, but may equally be used where the ABD EV is loaded with any cargo according to the invention. The immune effector may act to reduce immunogenicity of the ABD EV. In some embodiments, the immune effector functions stimulate immune inhibitors. In other embodiments, the immune effector functions inhibit immune stimulating molecules. In some embodiments, ABD-EV comprises molecules that stimulate immune inhibitors and molecules that inhibit immune stimulating molecules.

Exemplary immune effector molecules include, but are not limited to, one or more of CTLA4, B7-1, B7-2, PD-I, PD-L1, PD-L2, CD28, or VISTA. In some embodiments, the EV further comprises CTLA4 and PD-L1, CTLA and PD-L2, CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-LI and VISTA.

The immune effector molecule may form part of the ABD EV fusion construct, the cargo, the targeting moiety or may form part of an entirely separate fusion protein construct comprising an immune effector molecule fused to any EV protein according to the present invention.

The present invention also relates to ABD EVs which are loaded with small molecule cargos. The terms, “small molecule”, “small molecule cargo”, “small molecule drug” and “small molecule therapeutic” are used interchangeably herein and can be understood to relate to any molecular agent which may be used for the treatment, prophylaxis and/or diagnosis of a disease and/or disorder. Small molecule agents are normally synthesized via chemical synthesis means, but may also be naturally derived, for instance via purification from natural sources, or may be obtained through any other suitable means or combination of techniques. A brief definition of a “small molecule” is any organic compound with a molecular weight of less than 900 g/mol (Dalton) that may help to regulate a biological process. For the purposes of this invention, a small molecule may be substantially larger than 900 g/mol, for instance 1500 g/mol, 3000 g/mol, or occasionally even larger. Although many small molecules exhibit good oral bioavailability, many small molecule drugs need to be given intravenously or via some other route of administration, be it for pharmacokinetic, pharmacodynamic, toxicity and/or stability reasons. Examples of small molecules include anticancer agents such as doxorubicin, methotrexate, 5-fluorouracil or other nucleoside analogues such as cytosine arabinoside, proteasome inhibitors such as bortezomib, or kinase inhibitors such as imatinib or seliciclib, or non-steroidal anti-inflammatory drugs (NSAIDs) such as naproxen, aspirin, or celecoxib, antibiotics such as heracillin, or antihypertensives such as angiotensin-converting enzyme (ACE) inhibitors such as enalapril, ARBs such as candesartan, etc. The present invention is naturally applicable also to other small molecules without departing from the gist of the invention, as would be clear to a person skilled in the art.

In certain embodiments, the therapeutic cargo carried by the ABD EVs may be present on the inside of the EV, on the outside of the EV or in the membrane of the EV. The desired location of the therapeutic cargo will depend on the nature of the cargo and its mechanism of action, for instance, a membrane protein will preferably be located in the membrane of the EV, a decoy receptor will preferably be present on the surface of the EV, but a cargo designed to be delivered into the cytosol or nucleus of the recipient cell, such as a silencing RNA, will preferably be located inside the lumen of the EV.

The therapeutic cargo may be loaded passively into the EVs by the therapeutic cargo being present in the cytosol of the EV producing cells. Such passive loading applies, for instance, to nucleic acids, small molecules, viruses, soluble proteins or membrane proteins that are naturally loaded into the EVs.

In certain embodiments, the therapeutic cargo is actively loaded into the ABD EVs of the present invention. One form of active loading of cargos involves exogenous active loading, which involves cargo being loaded using any known exogenous loading method including: electroporation, transfection with transfection reagents such as cationic transfection agents, lipofectamine (RTM), conjugation of the cargo to a membrane-anchoring moiety, such as a lipid or cholesterol tail, or loading by means of a CPP, either in the form of a CPP-cargo conjugate or in the form of a CPP-cargo non-covalent complex. Again, this type of active loading may result in the therapeutic cargo protein being located on the inside of the EV, on the outside of the EV or located within the membrane of the EV.

In certain embodiments, the therapeutic cargo is actively loaded into the ABD EVs of the present invention by the use of fusion proteins. In this case, the therapeutic cargo carried by the EV forms part of the EV protein-ABD fusion protein or alternatively the therapeutic cargo carried by the EV forms part of an additional fusion protein with an EV-protein separate to the EV-protein-ABD fusion protein. In either case the therapeutic cargo protein may be fused into the fusion protein such that it is located on the inside of the EV, on the outside of the EV or located within the membrane of the EV. The presence of the EV protein in the fusion protein actively loads the therapeutic protein into the EV. The therapeutic cargo protein may be engineered so as to be fused to a single or multi-pass transmembrane protein at either the C or N terminus, so as to display the therapeutic protein on the surface of the EV or protect the therapeutic cargo within the EV. Any EV protein, as defined above, may be employed as a fusion partner for loading therapeutic protein cargos. EV proteins which may be employed to load cargo proteins into EVs may be transmembrane, but need not be. Where the therapeutic cargo forms part of an additional fusion protein with an EV-protein separate to the EV-protein-ABD fusion protein, it is clear that this fusion protein may include an EV protein which is membrane associated, rather than transmembrane. For instance, the fusion protein may employ EV proteins, which associate with the luminal/intravesicular surface of the EV membrane, ensuring that the therapeutic cargo is loaded into the lumen of the EV. Alternatively the cargo may be fused to an EV protein, which associates with the outer surface of the EV membrane. Particularly advantageous EV proteins include CD63, CD81, CD9, CD82, CD44, CD47, CD55, LAMP2B, ICAMs, integrins, ARRDC1, syndecan, syntenin, and Alix, as well as derivatives, domains, variants, mutants, or regions thereof.

The therapeutic protein may also be engineered into an extravesicular or intravesicular loop or loops of a multi-pass transmembrane protein, optionally wherein the multi-pass transmembrane protein is a tetraspanin, such as CD36, CD9, CD81 or any of TSPAN1-TSPAN33. Advantageously, cargo proteins may be incorporated into more than one of the loops of said multi-pass transmembrane protein and/or more than one cargo protein may be incorporated into each loop of said multi-pass transmembrane protein. More than one cargo protein may be incorporated into the transmembrane fusion protein without affecting the expression of the transmembrane protein. Without wishing to be bound by theory, the presence of more than one therapeutic cargo protein per transmembrane EV protein means that more cargo molecules can be bound to the EV per fusion protein expressed, which can increase the therapeutic potency of the EV and allows for different therapeutic cargos to be incorporated into the same EV, significantly increasing the versatility of the EVs produced.

In embodiments where the therapeutic cargo is actively loaded into the EVs utilizing a separate fusion protein construct, i.e. on an additional fusion construct, which does not comprise the albumin binding domain, said additional fusion construct may also further comprise: (i) at least one multimerization domain; (ii) at least one endosomal escape domain; (iii) at least one linker/spacer/scaffold sequence; (iv) at least one release domain or releasable linker capable of cleavage to release the therapeutic cargo; (v) at least one immune effector molecule; and/or (vi) at least one targeting moiety.

Suitable release domains according to the present invention include, but are not limited to, cis-cleaving sequences such as inteins, light induced monomeric or dimeric release domains such as Kaede, KikGR, EosFP, tdEosFP, mEos2, PSmOrange, the GFP-like Dendra proteins, Dendra and Dendra2, CRY2-CIBN, etc. Alternatively, nuclear localization signal (NLS) - nuclear localization signal-binding protein (NLSBP) (NLS-NLSBP) release system may be employed. Protease cleavage sites may also be incorporated into the fusion proteins for spontaneous release etc., depending on the desired functionality of the fusion polypeptide. In the case of nucleic acid cargos, specific nucleic acid cleaving domains may be included. Non-limiting examples of nucleic acid cleaving domains include, but are not limited to, endonucleases such as Cas6, Cas13, engineered PUF nucleases, site specific RNA nucleases etc.

Without wishing to be bound by theory, the inclusion of release domains can enable release of particular parts or domains from the original fusion polypeptide. This is particularly advantageous when the release of parts of the fusion polypeptide would increase bioactive delivery of the cargo and/or when a particular function of the fusion polypeptide works better when part of a smaller construct.

In a further embodiment, the EVs as per the present invention may comprise at least one targeting moiety to enable targeted delivery to a cell, tissue, organ, and/or compartment of interest. The targeting moiety may be comprised in a fusion polypeptide with an EV protein. In an advantageous embodiment, the targeting moiety is part of a fusion protein with a transmembrane EV protein to enable display of the targeting moiety on the surface of the EVs. In one embodiment, the targeting moiety may be comprised as part of the ABD EV protein fusion construct; alternatively the targeting moiety may be present as part of a separate fusion with an EV protein. Targeting moieties may be proteins, peptides, single chain fragments or any other derivatives of antibodies, obtainable from either humans or from non-human animals etc. The targeting moiety may form part of the ABD EV fusion construct or alternatively it may form part of a separate polypeptide construct which is comprised in the EV. EVs comprising targeting moieties may be produced using any of the methods disclosed herein.

The presence of the targeting moiety, combined with the improved biodistribution of ABD EVs, makes them especially useful in organ targeting. Without wishing to be bound by theory, once the ABD EV is coated in albumin it can remain in circulation much longer, avoiding uptake by the liver or cells of the immune systems, and thus is able to reach the desired target organ.

The targeting moiety may be fused to any EV protein according to the invention. The targeting moiety-EV protein fusion may be engineered so as to display the targeting moiety on the surface of the EV by fusion to the extravesicular portion of a single pass transmembrane protein. Alternatively, the targeting moiety may be engineered into an extravesicular loop or loops of a multi-pass transmembrane protein, optionally wherein the multi-pass transmembrane protein is a tetraspanin, such as CD63, CD9, CD81 or any of TSPAN1-TSPAN33.

In embodiments where the targeting moiety is present on a separate fusion protein construct, i.e. on an additional fusion construct which does not comprise the albumin binding domain, said additional fusion construct may also further comprise: (i) at least one multimerization domain; (ii) at least one endosomal escape domain; (iii) at least one immune effector molecule; and/or (iv) at least one linker/spacer/scaffold sequence.

Targeting moieties may be used to target the EVs to cell, subcellular locations, tissues, organs or other bodily compartments. Organs and cell types that may be targeted include, but are not limited to, the brain, neuronal cells, the blood brain barrier, muscle tissue, the eye, lungs, liver, kidneys, heart, stomach, intestines, pancreas, red blood cells, white blood cells including B cells and T cells, lymph nodes, bone marrow, spleen and cancer cells.

As would be appreciated by the skilled person, targeting can be achieved by a variety of means, for instance, the use of targeting peptides. Such targeting peptides may be anywhere from a few amino acids in length to several 100 s of amino acids in length, e.g. anywhere in the interval of about 3-100 amino acids, about 3-30 amino acids, about 5-25 amino acids, e.g. about 7 amino acids, about 12 amino acids, about 20 amino acids, etc. Targeting peptides of the present invention may also include full length proteins, such as receptors, receptor ligands, etc. Furthermore, the targeting peptides as per the present invention may also include antibodies and antibody derivatives, e.g. monoclonal antibodies, scFvs, other antibody domains, etc. Exemplary targeting moieties include brain targeting moieties such as rabies virus glycoprotein (RVG), nerve growth factor (NGF), melanotransferrin and the FC5 Peptide and muscle targeting moieties such as Muscle Specific Peptide (MSP). The targeting moiety may also be attached to the EV by way of an Fc binder fused to an EV protein. Targeting moieties which contain Fc domains such as antibodies or proteins which have been engineered to comprise an Fc domain may be attached to the surface of the EV by the presence of an Fc binder being incorporated into the targeting fusion construct. Examples of Fc binder proteins are: Protein A, Protein G, Protein A/G, Protein L, Protein LG, Z domain, ZZ domain, human FCGRI, human FCGR2A, human FCGR2B, human FCGR2C, human FCGR3A, human FCGR3B, human FCGRB, human FCAMR, human FCERA, human FCAR, mouse FCGRI, mouse FCGRIIB, mouse FCGRIII, mouse FCGRIV, mouse FCGRn, SPH peptide, SPA peptide, SPG2, SpA mimic 1, SpA mimic 2, SpA mimic 3, SpA mimic 4, SpA mimic 5, SpA mimic 6, SpA mimic 7, SpA mimic 8, SpA mimic 9, SpA mimic 10, Fc_{Y} mimic 1, Fc_{Y} mimic 2. In a specific embodiment the Fc binder is incorporated into the same multi-pass transmembrane protein as the ABD, for example, the ABD may be engineered into the 1^{st} loop and the targeting moiety may be engineered into the second loop or vice versa. In a more specific embodiment an EV may comprise at least one multi-pass transmembrane protein which has engineered into its loops both an ABD and an Fc binder acting as an anchor for a targeting moiety which comprises an Fc domain, for instance an antibody.

The present invention also relates to a population of EVs comprising at least one ABD present on the surface of the EV. In certain embodiments, the average number of ABDs per EV in the population of EVs according to the invention is above one ABD per EV, but it may also be below one per EV. Furthermore, in certain embodiments, in the population of EVs according to the invention, the average number of cargo molecules per EV is above or below one cargo molecule per EV. In another embodiment, in the population of EVs according to the invention, at least 5%, at least 10%, at least 20%, at least 50%, at least 70%, at least 75%, at least 80%, at least 85%, at least 90%, and/or at least 95% of all EVs comprise at least one ABD and optionally also at least one cargo molecule.

The present invention also relates to fusion proteins comprising at least one ABD and at least one EV protein, and polynucleotide constructs encoding such fusion proteins, as well as vectors, EVs and cells comprising such constructs.

The EV protein may optionally be a transmembrane EV protein or an EV protein associated with the outer membrane of the EV. Where the EV protein is a multi-pass transmembrane EV protein, the ABD may be engineered into an extravesicular loop or loops of the multi-pass transmembrane protein. In preferred embodiments, the multi-pass transmembrane EV protein of the fusion protein is a tetraspanin. The fusion proteins of the invention may advantageously comprise more than one ABD i.e. a plurality of ABDs. The plurality of ABDs may the same or different to one another.

Fusion proteins according to the present invention may further comprise: (i) at least one multimerization domain; (ii) at least one endosomal escape domain; (iii) at least one linker/spacer/scaffold sequence; (iv) at least one therapeutic cargo protein, optionally further comprising a release domain or releasable linker capable of cleavage to release the therapeutic cargo; (v) at least one immune effector molecule; and/or (vi) at least one targeting moiety.

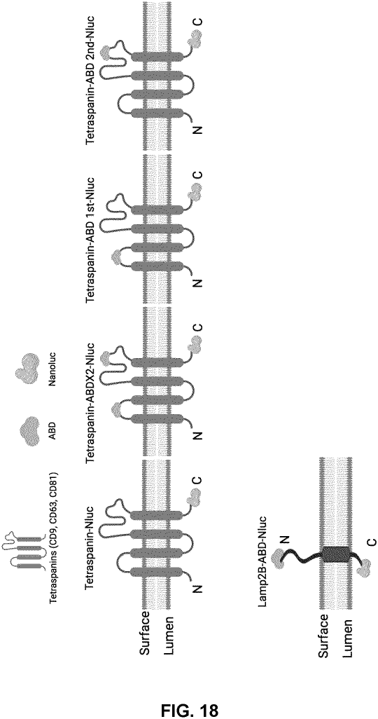

For clarity, the structures of a few ABD-EV protein fusion proteins are illustrated below and in FIG. 18:
EV protein-ABD
EV protein domain I-ABD-EV protein domain II
EV protein domain I-ABD-ABD-EV protein domain II
EV protein domain I-ABD-EV protein domain II-ABD-EV protein domain III
EV protein domain I-ABD-EV protein domain II-therapeutic cargo
Therapeutic cargo-EV protein domain I-ABD-EV protein domain II
EV protein domain I-ABD-EV protein domain II-targeting moiety
EV protein domain I-ABD-EV protein domain II-targeting moiety-EV protein domain III
EV protein domain I-ABD-EV protein domain II-therapeutic cargo-EV protein domain III

The present invention relates to EVs comprising the above identified fusion proteins.

Generally, EVs may be derived from essentially any cell source, be it a primary cell source or an immortalized cell line. The EV source cells may be any embryonic, fetal, or adult somatic stem cell types, including induced pluripotent stem cells (iPSCs) and other stem cells derived by any method, as well as any adult cell source. The source cells per the present invention may be selected from a wide range of cells and cell lines, for instance mesenchymal stem or stromal cells (obtainable from e.g. bone marrow, adipose tissue, Wharton’s jelly, perinatal tissue, chorion, placenta, tooth buds, umbilical cord blood, skin tissue, etc.), fibroblasts, amnion cells and more specifically amnion epithelial (AE) cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, erythrocytes, erythroid progenitors, chondrocytes, mesenchymal stromal cells (MSCs) of different origin, amnion cells, AE cells, CEVEC’s CAP^{®} cells, any cells obtained through amniocentesis or from the placenta, airway or alveolar epithelial cells, fibroblasts, endothelial cells, etc. Also, immune cells, such as B cells, T cells, NK cells, macrophages, monocytes, dendritic cells (DCs) are also within the scope of the present invention, and essentially any type of cell which is capable of producing EVs is also encompassed herein. The source cell may be either allogeneic, autologous, or even xenogeneic in nature to the patient to be treated, i.e. the cells may be from the patient him/herself or from an unrelated, matched or unmatched donor.

In particular, the present invention relates to cells which have been stably modified to comprise at least one monocistronic, bicistronic or multicistronic polynucleotide construct according to the invention (as defined above) encoding a fusion protein of an EV protein and at least one ABD protein. Such cells may be stably or transiently transfected with the polynucleotides according to the present invention to render them ABD-EV producing cells. Such cells may also be stably or transiently modified so as to include a construct encoding for a therapeutic cargo protein which optionally may form part of a fusion protein with an EV protein. Such cells may also be stably or transiently modified so as to include a construct encoding for a targeting moiety which comprises a fusion protein of the targeting moiety and an EV protein. The cells of the present invention may be of a monoclonal cell or a polyclonal cell line.

Preferred producer cells according to the present invention may include, but are not limited to, a HEK cell, a HEK293 cell, a HEK293T cell, an MSC, in particular a WJ-MSC cell or a BM-MSC cell, a fibroblast, an amnion cell, an AE cell, CEVEC’s CAP^{®} cells, a placenta-derived cell, a cord blood cell, an immune system cell, an endothelial cell, an epithelial cell or any other cell type, wherein said cells may be, for instance, adherent cells, suspension cells, and/or suspension-adapted cells.

The present invention relates to a method for producing the EVs according to the invention. The method for producing EVs comprises:
(i) introducing into an EV-producing cell at least one polynucleotide construct encoding an ABD-EV protein fusion construct; and
(ii) expressing said construct in the EV-producing cell, thereby generating an EV comprising ABD present on the surface of the EV.

Advantageously the production of the EVs by the present method results in endogenous loading of the ABD-EV protein fusion construct into the EV. Endogenous loading of proteins is essential for the proper protein confirmation and insertion of membrane proteins into the membrane as well as any necessary post translational modifications. The use of membrane proteins as the scaffold to anchor the ABD to the EV whilst also being present on the surface of the EV is essential to displaying the ABD on the EV whilst retaining all the intrinsic benefits of naturally derived EVs such as their native RNA and protein cargo and their immune-silent nature. Avoiding immunogenicity is important for therapeutics and is one of the intrinsic benefits of EVs over synthetic or in vitro produced therapeutics.

The method for producing the EVs may further comprise a step of loading the EV with at least one cargo molecule. Said cargo loading step may be by endogenous loading or exogenous loading.

Where the cargo loading step is an exogenous loading step, the loading step may comprise: loading of the cargo by any exogenous loading method including electroporation, microfluidics, transfection with transfection reagents such a cationic transfection agents, lipofectamine (RTM), conjugation of the cargo to a membrane anchoring moiety such as a lipid or cholesterol tail or loading by means of a CPP, either in the form of a CPP-cargo conjugate or in the form of a CPP-cargo non-covalent complex.

Where the cargo loading step is endogenous, the endogenous loading step may comprise introducing the cargo protein into the ABD-EV protein fusion construct or introducing into the EV-producing cell a further nucleic acid construct encoding the therapeutic cargo. It is also possible that a single nucleic construct might encode separately both the ABD-EV protein fusion protein as well as the cargo protein by the use of a bidirectional plasmid. Said therapeutic cargo construct may simply be expressed by the EV-producing cell and passively loaded into the EVs or the therapeutic cargo may be comprised as a fusion protein with an EV protein, so that the cargo protein, when translated, is endogenously and actively loaded into the EVs produced by the EV-producing cell.

The method for producing the EVs may further comprise a step of loading the EV with at least one targeting moiety. In this particular embodiment, the targeting moiety loading step is an endogenous loading step and may comprise introducing into the EV-producing cell a further nucleic acid construct encoding the targeting moiety, which is comprised as a fusion protein with an EV protein, so that, when translated, the targeting moiety is endogenously and actively loaded into the EVs produced by the EV-producing cell.

Without wishing to be bound by theory, the benefit of generating the cells as double or multiply stable cells is that a large library of producer cell lines can be generated quickly and easily by swapping out the cargo and/or targeting construct. Additionally, each separate construct can be placed under the control of a different promoter and thus the expression levels of the ABD, cargo and/or targeting moiety can be carefully and individually controlled. Alternatively, when the therapeutic cargo and/or the targeting moiety forms part of the ABD-EV protein fusion construct, the advantage of a single ABD-EV protein-cargo/targeting-moiety construct is that it only requires the cells to be made single stable, which results in the generation of simple and robust cell lines. The choice of a single, double or multiply stable cell line will depend upon the cargo and targeting moieties desired, their size and desired location on the EV.

Purification of EVs is achieved by any method including, but not limited to, techniques comprising liquid chromatography (LC), high-performance liquid chromatography (HPLC), bead-eluate chromatography, ionic exchange chromatography, spin filtration, tangential flow filtration (TFF), hollow fiber filtration, centrifugation, immunoprecipitation, flow field fractionation, dialysis, microfluidic-based separation, etc., or any combination thereof. In an advantageous embodiment, the purification of the EVs is carried out using a sequential combination of filtration (preferably ultrafiltration (UF), TFF or hollow fibre filtration) and affinity chromatography, optionally also including size exclusion LC or bead-eluate LC. Combining purification steps normally enhances the purity of the resulting samples and, in turn, leads to superior therapeutic activity. Further, as compared to ultracentrifugation (UC), which is routinely employed for purifying exosomes, sequential filtration-chromatography is considerably faster and possible to scale to higher manufacturing volumes, which is a significant drawback of the current UC methodology that dominates the prior art. Another advantageous purification method is TFF, which offers scalability and purity, and which may be combined with any other type of purification technique.

In some embodiments, the EVs of the present invention are isolated EVs. Accordingly, the present invention provides isolated EVs comprising at least one ABD present on the surface of the EV, wherein the ABD forms part of a fusion protein with an EV protein.

The present invention also relates to pharmaceutical compositions comprising at least one EV according to the invention and a pharmaceutically acceptable excipient or carrier.

The term, “excipient” or “carrier”, refers to an inert substance added to a pharmaceutical composition to further facilitate administration of a compound. The terms encompass any of the agents approved by a regulatory agency such as the FDA or EMEA or listed in the U.S. Pharmacopeia for use in animals, including humans, as well as any carrier or diluent that does not cause significant irritation to a subject and does not abrogate the biological activity and properties of the therapeutic cargo. Included are excipients and carriers that are useful in preparing a pharmaceutical composition and are generally safe and non-toxic.

Exemplary excipients include degradation or loss of activity stabiliser excipients including proteins such as HSA, polyols such as glycerol, sorbitol and erythritol, amino acids such as arginine, aspartic acid, glutamic acid, lysine, proline, glycine, histidine and methionine, polymers such as polyvinylpyrrolidone and hydroxypropyl cellulose, surfactants such as polysorbate 80, polysorbate 20 and pluronicF68, antioxidants such as ascorbic acid and alpha-tocopherol (vitamin E), buffers such as acetate, succinate, citrate, phosphate, histidine, tris(hydroxymethyl)aminomethane (TRIS), metal ion/chelators such as Ca²⁺, Zn²⁺ and EDTA, cyclodextrin-based excipients such as hydroxypropyl β-cyclodextrin and others such as polyanions and salts, stabilisers or bulking agents such as lactose, trehalose, dextrose, sucrose, sorbitol, glycerol, albumin, gelatin, mannitol and dextran, or preservatives such as benzyl alcohol, m-cresol, phenol, 2-phenoxyethanol.

The EVs as per the present invention may be administered to a human or animal subject via various different administration routes, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EVs, the cargo molecule in question, or the EV population as such.

It will be clear to the skilled artisan that, when describing medical and scientific uses and applications of the EVs, the present invention normally relates to a plurality of EVs, i.e. a population of EVs which may comprise thousands, millions, billions or even trillions of EVs. EVs may be present in concentrations such as about 10⁵, 10⁸, 10¹⁰, 10¹¹, 10¹², 10¹³, 10¹⁴, 10¹⁵, 10¹⁸, 10²⁵,10³⁰ EVs (often termed “particles”) per unit of volume or per unit of weight (for instance per ml or per L or per kg of body weight), or any other number larger, smaller or anywhere in between. In the same vein, the term “population”, which may e.g. relate to an EV comprising a certain ABD or cargo, can be understood to encompass a plurality of entities constituting such a population. In other words, individual EVs, when present in a plurality, constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of the cargo of interest, the ABD, any targeting moieties present on the EVs, the pharmaceutical formulation, etc.

It is envisaged that any dosage regime would be applicable to the ABD EVs of the invention. The dosage regime chosen will depend on the cargo being delivered by the ABD EVs and the disease to be treated and any additional therapies being administered which will be determined by the skilled physician.

It is envisaged that the ABD EVs of the present invention will be administered multiple times, i.e. more than once, but normally more than two times or potentially for chronic, long-term treatment (i.e. administered tens to hundreds to thousands of times). Preferably, if the cargo is an antigen that is being administered as a vaccine, the immunization schedule will involve two or more administrations of the polypeptide, spread out over several weeks. Similarly, if the cargo is e.g. an RNA agent such as an siRNA or mRNA or a protein such as an antibody or an enzyme or a transporter, the ABD EVs comprising the cargo in question will likely be administered more than once, normally multiple times as part of a chronic treatment regimen.

The present invention also relates to EVs according to the invention for use in medicine. The present invention also relates to a pharmaceutical composition according to the invention for use in medicine. The present invention also relates to a method of treatment comprising administering to a patient in need thereof, at least one effective amount of the EVs according to the invention or at least one effective amount of a pharmaceutical composition of the invention. The present invention also relates to a method of treating at least one disease, disorder and/or condition in a patient, the method comprising administering to the patient at least one effective amount of the EVs according to the invention or at least one effective amount of a pharmaceutical composition of the invention.

The medical use or method of treatment may be by delivery of any kind of cargo according to the invention. For instance, the medical use or treatment may be by delivery of functional proteins as protein replacement therapy, delivery of mRNA encoding functional proteins to also act as a protein replacement therapy. Such a protein replacement therapy may, for instance, be ERT for diseases caused by inborn errors in metabolism, such as phenylketonuria (PKU), urea cycle disorders, or lysosomal storage disorders. The medical use or treatment may be by delivery of gene silencing RNAs, splice switching RNAs, or CRISPR-Cas9 for gene editing. The medical use or treatment may be gene therapy by delivery of plasmid DNA, mini-circles or viral gene therapies such as AAVs or lentiviruses. The medical use or treatment may be by presentation of an antigen or neoantigen for immunotherapy, in effect acting as a vaccine to induce an immune response. For instance, the EV may act by delivery and/or presentation of a tumour antigen for cancer immunotherapy, or viral, bacterial or fungal antigens for immunization against pathogens. The medical use or treatment may be by delivery of small molecules, antibodies or antibody-drug conjugates capable of mediating a therapeutic effect once delivered into a cell or the extracellular matrix. In one embodiment, the medical use or treatment may be effected by the EVs comprising more than one type of therapeutic cargo, i.e. the therapeutic cargo may be a mixture of protein, nucleic acid, virus, viral genome, antigen and/or small molecule.

Importantly, the present invention relates to use of the EVs or pharmaceutical composition described herein in the prophylaxis and/or treatment and/or alleviation of a variety of diseases, typically via the delivery of essentially any type of drug cargo, such as for instance: a nucleic acid such as an RNA molecule, a DNA molecule or a mixmer, mRNA, antisense or splice-switching oligonucleotides, siRNA, shRNA, miRNA, pDNA, supercoiled or unsupercoiled plasmids, mini-circles, peptides or proteins including transporters, enzymes, receptors such as decoy receptors, membrane proteins, cytokines, antigens and neoantigens, ribonuclear proteins, nucleic acid binding proteins, antibodies, nanobodies, antibody fragments, antibody-drug conjugates, small molecule drugs, gene editing technology such as CRISPR-Cas9, TALENs, meganucleases, or vesicle-based cargos such as viruses (e.g. AAVs, lentiviruses, etc.). In one embodiment, the cargo may be a mixture of protein, nucleic acid, virus, viral genome, antigen and/or small molecule.

Non-limiting examples of diseases and conditions that are suitable targets for treatment using the EVs and pharmaceutical compositions described herein include the following non-limiting examples: autoimmune diseases (such as celiac disease, Crohn’s disease, diabetes mellitus type 1, Graves’ disease, inflammatory bowel disease, multiple sclerosis, psoriasis, rheumatoid arthritis, systemic lupus erythematosus, ulcerative colitis, ankylosing spondylitis, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis), stroke, acute spinal cord injury, vasculitis, Guillain-Barré syndrome, acute myocardial infarction, acute respiratory distress syndrome (ARDS), sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, Duchenne muscular dystrophy (DMD) and other muscular dystrophies, in-born errors of metabolism including disorders of carbohydrate metabolism, e.g., G6PD deficiency galactosemia, hereditary fructose intolerance, fructose 1,6-diphosphatase deficiency and the glycogen storage diseases, disorders of organic acid metabolism (organic acidurias), such as alkaptonuria, 2-hydroxyglutaric acidurias, methylmalonic or propionic acidemia, multiple carboxylase deficiency, disorders of amino acid metabolism, such as PKU, maple syrup urine disease, glutaric acidemia type 1, aminoacidopathies, e.g., hereditary tyrosinemia, nonketotic hyperglycinemia, and homocystinuria, hereditary tyrosinemia, Fanconi syndrome, Primary Lactic Acidoses e.g., pyruvate dehydrogenase, pyruvate carboxylase and cytochrome oxidase deficiencies, disorders of fatty acid oxidation and mitochondrial metabolism, such as short, medium, and long- chain acyl-CoA dehydrogenase deficiencies also known as Beta-oxidation defects, Reye’s syndrome, medium-chain acyl-coenzyme A dehydrogenase deficiency (MCADD), mitochondrial encephalopathy lactic acidosis and stroke-like episodes (MELAS), myoclonic epilepsy with ragged red fibers (MERRF), pyruvate dehydrogenase deficiency, disorders of porphyrin metabolism such as acute intermittent porphyria, disorders of purine or pyrimidine metabolism such as Lesch-Nyhan syndrome, disorders of steroid metabolism such as lipoid congenital adrenal hyperplasia, congenital adrenal hyperplasia, disorders of mitochondrial function such as Kearns-Sayre syndrome, disorders of peroxisomal function such as Zellweger syndrome and neonatal adrenoleukodystrophy, congenital adrenal hyperplasia or Smith-Lemli-Opitz, Menkes syndrome, neonatal hemochromatosis, urea cycle disorders such as N-acetylglutamate synthase deficiency, carbamoyl phosphate synthetase deficiency, OTC deficiency, citrullinemia (deficiency of argininosuccinic acid synthase), argininosuccinic aciduria (ASA; deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1), orotic aciduria (deficiency in the enzyme, UMPS), all of the lysosomal storage diseases, for instance alpha-mannosidosis, beta-mannosidosis, aspartylglucosaminuria, cholesteryl ester storage disease, cystinosis, Danon disease, Fabry disease, Farber disease, fucosidosis, galactosialidosis, Gaucher disease Type I, Gaucher disease Type II, Gaucher disease Type III, GM1 gangliosidosis Type I, GM1 gangliosidosis Type II, GM1 gangliosidosis Type III, GM2 - Sandhoff disease, GM2 - Tay-Sachs disease, GM2 - gangliosidosis, AB variant, mucolipidosis II, Krabbe disease, lysosomal acid lipase deficiency, metachromatic leukodystrophy, MPS I - Hurler syndrome, MPS I -Scheie syndrome, MPS I - Hurler-Scheie syndrome, MPS II - Hunter syndrome, MPS IIIA - Sanfilippo syndrome Type A, MPS IIIB - Sanfilippo syndrome Type B, MPS IIIB - Sanfilippo syndrome Type C, MPS IIIB - Sanfilippo syndrome Type D, MPS IV Morquio Type A, MPS IV - Morquio Type B, MPS IX - hyaluronidase deficiency, MPS VI - Maroteaux-Lamy, MPS VII - Sly syndrome, Mucolipidosis I - sialidosis, mucolipidosis IIIC, mucolipidosis Type IV, mucopolysaccharidosis, multiple sulfatase deficiency, neuronal ceroid lipofuscinosis T1, neuronal ceroid lipofuscinosis T2, neuronal ceroid lipofuscinosis T3, neuronal ceroid lipofuscinosis T4, neuronal ceroid lipofuscinosis T5, neuronal ceroid lipofuscinosis T6, neuronal ceroid lipofuscinosis T7, neuronal ceroid lipofuscinosis T8, neuronal ceroid lipofuscinosis T9, neuronal ceroid lipofuscinosis T10, Niemann-Pick disease Type A, Niemann-Pick disease Type B, Niemann-Pick disease Type C (NPC), Pompe disease, pycnodysostosis, Salla disease, Schindler disease and Wolman disease, etc., cystic fibrosis, primary ciliary dyskinesia, pulmonary alveolar proteinosis, arthrogryposis-renal dysfunction-cholestasis (ARC) syndrome, Rett syndrome, neurodegenerative diseases including Alzheimer’s disease, Parkinson’s disease, GBA associated Parkinson’s disease, Huntington’s disease and other trinucleotide repeat-related diseases, prion diseases, dementia including frontotemporal lobe dementia, amyotrophic lateral sclerosis (ALS), motor neuron disease, multiple sclerosis, cancer-induced cachexia, anorexia, diabetes mellitus type 2, and various cancers.

The present invention provides EVs comprising at least one ABD, wherein the EVs accumulate within a tumor and/or the lymph nodes at a level greater than the level of accumulation exhibited by otherwise identical EVs that lack the at least one ABD. Specifically the level of tumor and/or lymph node accumulation as compared to EVs lacking ABD may be at least 2-fold, or at least 3-fold, or at least 4-fold, or at least 5 fold, or at least 6 fold, or at least 7 fold or at least 8 fold, or at least 9 fold, or at least 10 fold, or at least 20 fold or more.

The present invention is especially advantageous for the treatment of cancers due to the accumulation of ABD EVs in tumors; specifically the treatment of cancer by immunotherapy, due to accumulation in the lymph nodes.

Specifically, it is envisaged that the present invention is useful in the treatment of cancer by cancer immunotherapy, i.e. the presentation of cancer antigens on the surface of ABD EVs so that those antigens raise an immune response against the cancer antigen. Virtually all types of cancer are relevant disease targets for the present invention, for instance, acute lymphoblastic leukemia (ALL), acute myeloid leukemia, adrenocortical carcinoma, AIDS-related cancers, AIDS-related lymphoma, anal cancer, appendix cancer, astrocytoma, cerebellar or cerebral, basal-cell carcinoma, bile duct cancer, bladder cancer, bone tumor, brainstem glioma, brain cancer, brain tumor (cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt’s lymphoma, carcinoid tumor (childhood, gastrointestinal), carcinoma of unknown primary, central nervous system lymphoma, cerebellar astrocytoma/malignant glioma, cervical cancer, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, extracranial germ cell tumor, extragonadal germ cell tumor, extrahepatic bile duct cancer, eye Cancer (intraocular melanoma, retinoblastoma), gallbladder cancer, gastric (stomach) cancer, gastrointestinal carcinoid tumor, gastrointestinal stromal tumor (GIST), germ cell tumor (extracranial, extragonadal, or ovarian), gestational trophoblastic tumor, glioma (glioma of the brain stem, cerebral astrocytoma, visual pathway and hypothalamic glioma), gastric carcinoid, hairy cell leukemia, head and neck cancer, heart cancer, hepatocellular (liver) cancer, Hodgkin lymphoma, hypopharyngeal cancer, intraocular melanoma, islet cell carcinoma (endocrine pancreas), Kaposi sarcoma, kidney cancer (renal cell cancer), laryngeal cancer, leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia)), lip and oral cavity cancer, liposarcoma, liver cancer (primary), lung cancer (non-small cell, small cell), lymphomas, AIDS-related lymphoma, Burkitt lymphoma, cutaneous T-cell lymphoma, Hodgkin lymphoma, non-Hodgkin, medulloblastoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic/myeloproliferative diseases, yelogenous leukemia, chronic myeloid leukemia (acute, chronic), myeloma, nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic islet cell cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, pleuropulmonary blastoma, prostate cancer, rectal cancer, renal cell carcinoma (kidney cancer), retinoblastoma, rhabdomyosarcoma, salivary gland cancer, sarcoma (Ewing family of tumors sarcoma, Kaposi sarcoma, soft tissue sarcoma, uterine sarcoma), Sézary syndrome, skin cancer (nonmelanoma, melanoma), small intestine cancer, squamous cell, squamous neck cancer, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine cancer, uterine sarcoma, vaginal cancer, vulvar cancer, Waldenström macroglobulinemia, and/or Wilm’s tumor.

The present invention is also specifically advantageous for the treatment of brain and CNS disorders due to the increased biodistribution of ABD EVs to the brain. The present invention provides EVs comprising at least one ABD, wherein the EVs accumulate within the brain at a level greater than the level of accumulation exhibited by otherwise identical EVs that lack the at least one ABD. Specifically the level of tumor accumulation as compared to EVs lacking ABD may be at least 2-fold, or at least 3-fold, or at least 4-fold, or at least 5 fold, or at least 6 fold, or at least 7 fold or at least 8 fold, or at least 9 fold, or at least 10 fold, or at least 20 fold or more. The present invention specifically relates to EVs with ABD present on the surface of the EV, which are loaded with any type of cargo according to the invention, but preferably a nucleic acid cargo, such as silencing RNAs, such as siRNAs, which target RNAs known to be involved with neurodegenerative diseases such as Alzheimer’s disease, Parkinson’s disease, Huntington’s disease, spinocerebellar ataxia, ALS, frontal temporal dementia, motor neuron disease, multiple sclerosis, Wallerian degeneration and bullous retinoschisis, Goldberg-Shprintzen syndrome, kuru, autoimmune GFAP astrocytopathy, methyl CpG binding protein 2 (MECP2) duplication syndrome, aquaporin-4 (AQP4)-astrocytopathy, familial pain syndromes such as erythromelalgia, paroxysmal extreme pain disorder and congenital insensitivity to pain, Pelizaeus-Merzbacher disease, prion diseases including Creutzfeldt-Jakob disease (CJD), Gerstmann-Sträussler-Scheinker syndrome (GSS), and fatal familial insomnia (FFI), leukodystrophies including demyelinating, adult-onset, autosomal dominant and leukodystrophy.

The present invention specifically relates to EVs with ABD present on the surface, which are further loaded with any therapeutic cargo according to the invention, the therapeutic cargo may be both protein and/or nucleic acid cargos, for the treatment of DMD, diseases caused by in-born errors of metabolism including lysosomal storage disorders including NPC and Pompe disease, urea cycle disorders such as ASA and citrullinemia and OTC deficiency, metachromatic leukodystrophy and PKU.

Increasing the shelf-life of complex products, such as therapeutic exosomes, is vitally important to enabling the enormous therapeutic benefits of EVs to be realised. Furthermore, EVs are known to be structurally susceptible to damage due to the exposure of vulnerable phosphatidylserine to repeated freeze-thaw cycles. The present invention aims to overcome these problems.

The present invention also relates to a method of improving half-life of EVs in storage, the method comprising: a) obtaining EVs according to the invention, and b) formulating said EVs in a storage or formulation buffer which comprises albumin. Any form of albumin may be utilised, optionally the albumin is recombinant albumin, human albumin, serum albumin, HSA, recombinant serum albumin, recombinant HSA, Albunorm® or any fragment or domain therefore that binds to the ABD of the invention. HSA has a particularly long half-life, so is preferred. This fact also means that the half-life extensions observed in the mouse data in this application can be expected to be improved still further when the ABD-EVs of the present invention are tested in humans.

Without wishing to be bound by theory, the presence of ABD on the surface of the EVs, combined with the presence of albumin in the storage/formulation buffer, has the advantage of allowing the albumin to bind to the ABD on the surface of the EVs, thus creating a protective shield around the EVs. This possibly prevents aggregation of EVs, but also encourages the formation of larger heterogenous nanoparticles, which protect the EVs from damaging freeze-thaw cycles and shear stress during processing. This results in a much more robust EV population with a long shelf life. Without wishing to be bound by theory, it is believed that the albumin coat prevents unwanted interaction of the EV membrane with the walls of the containers, meaning that more EVs are retrieved after storage and additionally those that are retrieved are of high quality.

The additional benefit of the use of ABD and albumin in extending the shelf life of these compositions is that the albumin-coated EVs can be directly administered to patients and the albumin will then function to increase the half-life of the EV in circulation. Thus by only one modification to the EVs, the inclusion of the ABD domain, the EVs are made more robust during storage and formulation. This means they have greater therapeutic efficacy, loss of EVs due to adherence to container surfaces is reduced and, importantly, when those EVs are then administered to a patient, they also have increased circulation time in vivo.

The present invention provides EVs comprising at least one ABD, wherein the EVs exhibit a shelf-life that is at least 5%, at least 10%, at least 20%, at least 30%, at least 50%, at least 70%, at least 90% or more than the shelf-life exhibited by otherwise identical EVs that lack at the least one ABD. The EVs may exhibit a shelf-life that is weeks, months or years greater than the shelf-life exhibited by otherwise identical EVs that lack at least one ABD, for instance, the shelf-life may be increased by 5 weeks, 10 weeks, 20 weeks, 1 month, 3 months, 6 months, 9 months, 1 year, 2 years or more.

It is important to note that albumin does not contain blood-derived impurities that otherwise could activate unwanted cell pathways and result in a product which is unable to meet regulatory approval requirements regarding host cell protein levels. Recombinant albumin can help maintain the safety and efficacy of final drug products during storage by avoiding the presence of host cell proteins.

Precoating of ABD-EVs with albumin, such as recombinant albumin, present in the storage or formulation buffer has the additional benefit of creating a pre-coated form of the ABD-EV. Such a pre-coated ABD-EV can be administered to a patient and there be no delay in the ABD-EV acquiring the albumin from the patient’s circulation required to extend the half-life of the ABD-EV. Such a pre-coated ABD-EV will therefore display significantly reduced uptake of the ABD-EVs at the injection site (i.e. a reduction in off target uptake). This, again, results in more of the administered dose of ABD-EV being present in circulation for longer, thus increasing the therapeutic efficacy of the pre-coated ABD-EVs.

The present invention is also directed to nanoparticle complexes comprising an ABD-EV according to the invention, wherein some or all of the ABDs of the ABD-EVs are bound to albumin (i.e. the ABD-EVs are decorated or pre-coated on the surface of the EV with at least one type of albumin). The present invention also relates to pharmaceutical compositions comprising the nanoparticle complexes of the invention combined with a pharmaceutically acceptable excipient or carrier.

In further aspects, the present invention thus also relates to EVs, EV-protein complexes, and/or pharmaceutical compositions comprising such EVs and EV-protein complexes for use in medicine, preferably in the treatment of diseases which would benefit from antibody- or Fc domain-containing protein-based treatment, antibody-drug conjugate (ADC)-based treatment, and/or antibody-mediated targeting.

As described above, the precoating of ABD-EVs with albumin, such as recombinant albumin, has the additional benefit of creating a pre-coated form of the ABD-EV, which does not rely on binding of albumin from the patient’s own blood. This speeds up the protective effect of the albumin, which, in turn, increases the half-life and thus increases the therapeutic efficacy of the ABD-EVs. Another significant benefit of employing nanoparticle complexes is that the strength of the binding of the albumin to the ABD can be pre-defined by genetic engineering of the ABD binding site and genetic engineering of the albumin used. This allows for high, medium or weak strength associations between the ABD and albumin, which gives the nanoparticle complex a modulatable binding affinity, so half-life can be controlled in a very refined manner.

The present invention also pertains to affinity chromatography isolation and purification of the EVs of the invention, wherein the EVs are engineered to enable highly specific binding to e.g. chromatography matrices, and optionally subsequent elution. EVs according to the present invention comprise ABD proteins on the surface of the EV. Purification of said EVs by affinity purification is possible utilising affinity of the ABD present on the EVs for its binding partner, albumin.

Conventional methods to prepare and isolate EVs (e.g. exosomes) involve a series of differential centrifugation steps to separate the vesicles from cells or cell debris present in the culture medium into which the EVs are released by EV-producing cells. Typically, series of centrifugations at e.g. 300 g, 10,000 g and 70,000 g or 100,000 g are applied, upon which the resulting pellet at the bottom of the tube is resuspended to a fraction of its original volume with a saline solution to constitute a concentrated EV or exosome solution. However, these methods are essentially unsuitable for clinical applications for a number of reasons: (1) the extended length of time needed for the entire process, (2) issues around scale-up and validation in a GMP environment, (3) significant risk of contamination by cell debris, resulting in host cell protein contamination at levels unacceptable for regulatory approval, (4) poor reproducibility due to operator variability, (5) aggregation of EVs/exosomes resulting from pelleting of the vesicles, (6) low recovery at end of processing, and (7) negative impact on vesicle morphology and thereby biodistribution and activity. There is therefore a need for improved methods of preparing membrane vesicles, suitable with industrial constraints and allowing production of vesicle preparations of therapeutic quality. To that end, International Patent Publication No. WO 2000/044389 discloses methods for preparing membrane vesicles from biological samples through chromatographic techniques, such as anion exchange chromatography and/or gel permeation chromatography. However, there is room for significant improvement over said disclosures, especially as the EV therapeutics field advances toward clinical translation and impact of EV-based therapies. The previously known methods for purifying exosomes are not ideally suited to large scale production and scale up that would be necessary for commercial production of EV therapeutics. The present invention allows much larger scale purification of engineered exosomes with high affinity than would be achievable with previously known methods.

The present invention achieves these, and other, objectives by utilizing chromatography matrices comprising albumin domains, which have affinity for the EVs of the present invention (which are engineered to comprise ABD polypeptides on the surface of the EVs). As such, the present invention thus relates to various aspects and embodiments surrounding processes for isolating and/or purifying EVs. Advantageously, the ABD is used to purify the EVs and additionally, once the ABD-EVs are produced and purified, the ABD present on the surface endows the EVs with increased shelf life and a prolonged half-life. It is clear that the addition of ABD to the EVs has the benefit of a dual function of allowing both purification and improved half-life and shelf-life. The addition of ABD therefore generates extremely versatile EVs with only a single genetic engineering step.

The present invention provides EVs comprising at least one ABD, wherein the EVs exhibit a half-life in a human that is at least 5%, at least 10%, at least 20%, at least 30%, at least 50% or more than the half-life exhibited by otherwise identical EVs that lack at least one ABD. For instance the EVs may exhibit a half-life in a human that is at least 10 minutes, at least 20 minutes, at least 30 minutes, at least one hour, at least two hours, at least three hours greater than the half-life exhibited by otherwise identical EVs that lack at least one ABD.

The affinity purification method of the present invention comprises the steps of: (i) contacting a medium comprising the ABD EVs with a chromatography matrix comprising albumin or a fragment or domain thereof, (ii) allowing the ABD EVs of the invention to adsorb to the albumin or fragment/domain thereof, and (iii) eluting the ABD EVs by passing across the chromatography matrix a medium that releases the ABD EVs from the albumin or fragment or domain thereof. As above-mentioned, the EVs of the present invention are engineered to comprise and display on their surface ABDs such as the ABDs given in SEQ ID NOs:1-4.

The principle of this process is what is known as affinity purification and/or affinity chromatography, i.e. purification of a particular target solute (in this case EVs, such as exosomes) from a complex biological fluid containing various types of solutes, based on the specific interaction between a generic ligand and a generic corresponding receptor, in this case an albumin or a fragment or domain thereof and an ABD. The albumin or fragment/domain thereof is thus attached to a stationary phase, whereas the ABD polypeptide is present on the EVs, which are comprised in the liquid phase, e.g. cell culture medium. The processes and methods of the present invention are easily applied to any type of cell culture medium, and various cell culture medium used for both adherent and suspension cells have been tested in the affinity chromatography methods of the present invention, for instance, RPMI, EMEM, DMEM, MEM, PMEM, PEM, Opti-MEM, IMDM, Advanced DMEM, McCoy’s medium, medium with or without additives such serum, antibiotics, nutrients, etc.

In a further embodiment, the process may comprise triggering release of the EVs from the albumin or fragment/domain thereof by exposing the albumin - ABD bond to a medium with a suitable pH. This is achieved by running the EV-containing medium (i.e. the liquid phase) through e.g. a chromatography column comprising, as stationary phase, a chromatography matrix having attached to it albumin or fragment/domain thereof, letting the ABD polypeptides of the EVs adsorb to the albumin or fragment/domain thereof present on the matrix, and then running a solution with a suitable pH through the chromatography column. The pH of the solution that is intended to trigger release of the EVs from the column may be below pH 8, below pH 7, or below pH 6. Both the process of capturing the EVs and the process of releasing the EVs may be repeated multiple times, e.g. anywhere from once to up to e.g. 500 times.

In further embodiments, the albumin or fragment/domain thereof may be attached to the chromatography matrix via different types of chemical and biochemical linkages and bonds. Covalent bonds between the matrix and the protein comprising the albumin or fragment/domain thereof, e.g. HSA, may be conventional amide bonds, disulfide bonds, ether bonds, esther bonds, thio-ether bonds, thio-esther bonds, glutathione-GST interactions, streptavidin-biotin interaction, etc. The matrix may be chemically activated to facilitate binding to the albumin protein or fragment/domain thereof using chemical conjugation moieties such as N-hydroxysuccinimide (NHS; for NHC-EDC/EDAC coupling), thiols, cyanogen bromide (CNBr), epoxy, thiopropyl, primary amines, sulfhydryls, carboxylic acids, aldehydes, iodoacetyl, azlactones, carbonyldiimidazole (CDI), maleimide, etc., as is well known to a person skilled in the art.

In preferred embodiments, the processes of the present invention are carried out in chromatography columns, which comprise the chromatography matrix comprising the albumin or fragment/domain thereof.

The chromatography matrix for use in capturing ABD EVs may consist of essentially any type of material suitable as a stationary chromatography phase. Non-limiting examples include one or more of agarose, dextran, lectin, heparin, cellulose, starch, dextran, agar, agarose, poly(meth)acrylate, polyacrylamide, polysulfone, a polyvinyl polymer, polystyrene, silica, alumina, zirconium oxide, titanium oxide, polysaccharide-mineral structure, polysaccharide-synthetic polymer, synthetic polymer-mineral structure, or any combination thereof. The matrix may be in the form of beads, fibers, irregularly shaped particles, membranes, flat structure, porous mineral materials or essentially any type of suitable stationary phase. Naturally, the albumin or fragment/domain thereof attached to the matrix may also be directly attached to various surfaces using chemical bonds and linkers. This could be particularly useful for methods such as e.g. surface plasmon resonance.

The affinity purification methods as per the present invention may further comprise additional EV purification step(s), which may be carried out prior to the affinity capture step(s) of the present invention. Suitable purification methods are as disclosed herein in connection with the second aspect of the invention.

Importantly, as above-mentioned, the affinity purification of ABD EVs may be run multiple times, essentially indefinitely, but at least anywhere between 2 and 500 times. Sequential purification of albumin-binding EVs enables exogenous drug loading between purification steps. For instance, albumin-binding EVs may be purified directly from the conditioned medium (CM) of the EV-producing cell source, followed by an exogenous drug loading step, and yet another round of purification. Schematically, this can be illustrated as follows:
Secretion of EVs into cell culture medium by EV-producing cells;
Affinity purification of EVs using the processes and methods of the present invention;
Drug loading (for instance, loading of siRNA into EVs/onto the surface of EVs by electroporation or transfection reagent);
Affinity purification of EVs using the processes and methods of the present invention.

The additional exogenous loading step may involve cargo being loaded using any known exogenous loading method including: electroporation, transfection with transfection reagents such a cationic transfection agents, lipofectamine (RTM), conjugation of the cargo to a membrane-anchoring moiety, such as a lipid or cholesterol tail, or loading by means of a CPP, either in the form of a CPP-cargo conjugate or in the form of a CPP-cargo non-covalent complex.

Any of the above aspects can be combined with any other aspect.

### EXAMPLES

### Example 1: Expression of Constructs in Producer Cells

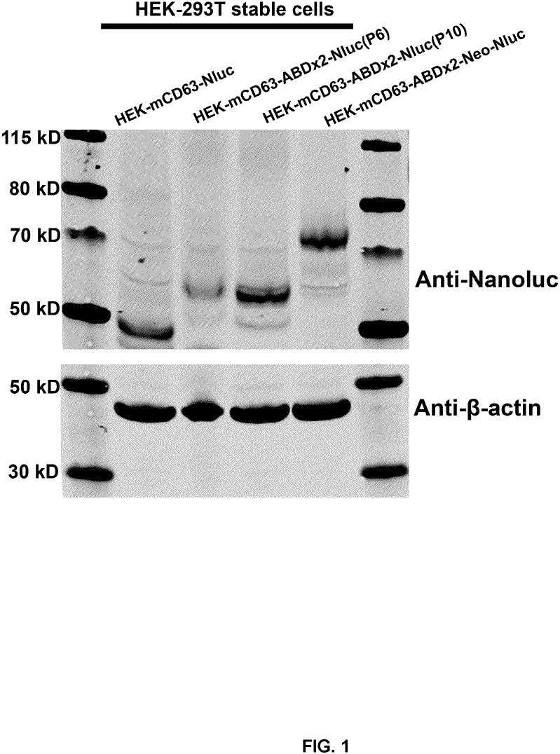

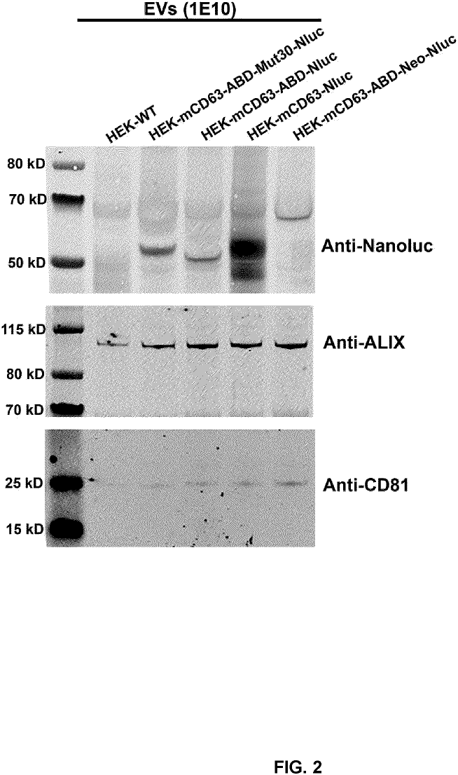

Nucleic acid fusion constructs encoding the fusion proteins described in the Key below were designed and introduced into EV producing cells using technology as disclosed herein, so that those cells expressed the fusion protein. This fusion protein was then incorporated into the EVs produced by the producer cell, due to the presence of the EV protein in the fusion construct. Expression of constructs in stably transduced HEK-293T producer cells was tested by western blot. Nanoluc and actin primary antibodies were used respectively for detection. The data shown in FIG. 1 show that all the constructs expressed well in HEK-293T stable cells.

### Key:

mCD63-Nluc (SEQ ID NO:5): control fusion protein comprising fusion protein of an exosome protein (CD63) and nanoluc reporter (lacking ABD).
mCD63-ABDX2-Nluc (SEQ ID NO:6): fusion protein of exosomal protein (CD63) with ABDs engineered so as to be present on the surface of the EVs and expressing nanoluc reporter (P6= passage 6, P10 = passage 10).
mCD63-ABDX2-Neo-Nluc (SEQ ID NO:9): fusion protein of exosomal protein (CD63) with ABDs engineered so as to be present on the surface of the EVs and expressing nanoluc reporter additionally comprising tumor antigens (neoantigen = 6 tumor antigens).

Neoantigens are included to deliver the neoantigen cargo for use as a vaccine to treat cancer by immunotherapy. The addition of neoantigens into the construct shows that additional sequences and motifs can be added into the same CD63-ABD construct without affecting the expression of the construct. Any other therapeutic protein could be inserted into the fusion construct in place of the neoantigens in this example.

All constructs expressed well in HEK-293T stable cells. The expression of mCD63-ABDX2-Nluc construct did not decrease after several passages showing that the constructs are stable over a number of passages despite significant genetic engineering, which is known can affect the expression of certain expression cassettes after several passages. The ABD used in the constructs of Example 1 was ABD035 (SEQ ID N0:4).

### Example 2: Expression of Constructs in EV Fraction

Once the expression of constructs in stably transduced HEK-293T producer cells was established, the expression of the constructs in the EV fraction after purification was then tested by western blot.

### Key:

HEK-WT: EVs from HEK-293T WT cells as negative control.
mCD63-ABDX2-Mut30-Nluc (SEQ ID NO:10): fusion protein of exosomal protein (CD63) with ABD engineered so as to be present on the surface of the EVs and expressing nanoluc reporter. Additionally comprising tumor antigen (Mut30).
mCD63-ABDX2-Nluc (SEQ ID NO:6): fusion protein of exosomal protein (CD63) with ABDs engineered so as to be present on the surface of the EVs and expressing nanoluc reporter.
mCD63-Nluc (SEQ ID NO:5): control fusion protein comprising fusion protein of an exosome protein (CD63) and nanoluc reporter (lacking ABD).
mCD63-ABDX2-Neo-Nluc (SEQ ID NO:9): fusion protein of exosomal protein (CD63) with ABD engineered so as to be present on the surface of the EVs and expressing nanoluc reporter additionally comprising tumor antigens (neoantigen = 6 tumor antigens).

All the constructs expressed well in EVs purified from HEK-293T stable cells. The expression of the constructs did not decrease after several passages.

Nanoluc, ALIX and CD81 primary antibodies were used respectively for detection (ALIX and CD81 being exosomal marker proteins). The ABD used in the constructs of Example 2 was ABD035 (SEQ ID NO: 4)

### Example 3: Improved Half-Life of ABD EVs in Vivo

The half-life of ABD EVs was tested in vivo in NMRI mice compared to EVs lacking an ABD domain. EVs engineered to express ABD on the surface plus luminescent nanoluc reporter (mCD63-ABDX2-Nanoluc (SEQ ID NO:6)) and EVs engineered to only express nanoluc (mCD63-Nanoluc (SEQ ID NO:5)) were compared. The ABD used in the constructs of Example 3 was ABD035 (SEQ ID NO: 4). 6 NMRI mice per group were used and EVs were injected intravenously at a dose of 1e11/mouse. The number of EVs remaining in circulation at a certain time point is calculated based on relative light unit (RLU)/injected EV number (Total RLU/1E11). From this, the percentage of injected EVs in plasma is derived.

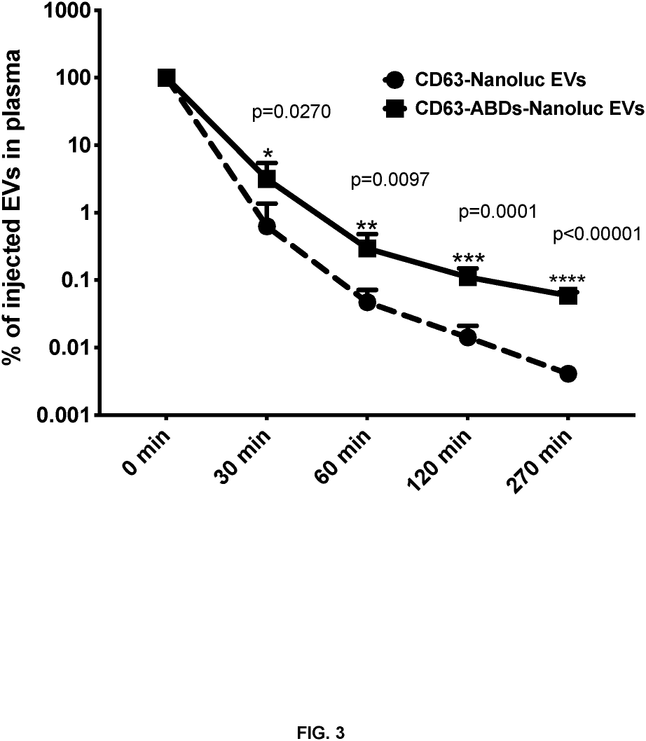

The data in FIG. 3 show that the presence of ABD significantly increased the half-life of EVs in circulation, by making them more stable at least up to the 4.5 h time point.

The presence of ABD on the surface of EVs resulted in more than 14 times more EVs in the circulation compared to the control group which, as can be seen from FIG. 3, led to an impressively large increase in the half-life of EVs in circulation.

### Example 4: Biodistribution of ABD EVs Compared to Control EVs

It was predicted that the improved half-life of EVs would impact on the biodistribution of those EVs. In order to investigate the biodistribution of ABD EVs (mCD63-ABDx2-NanoLuc (SEQ ID NO:6)) compared to control EVs (mCD63-NanoLuc (SEQ ID NO:5)), blood was sampled and the internal organs were harvested 270 min after injection. The ABD used in the constructs of Example 4 was ABD035 (SEQ ID NO: 4). Total RLUs in each organ or plasma were measured and the percentage of injected EVs was calculated based on RLU/injected EV number (Total RLU/1E11).

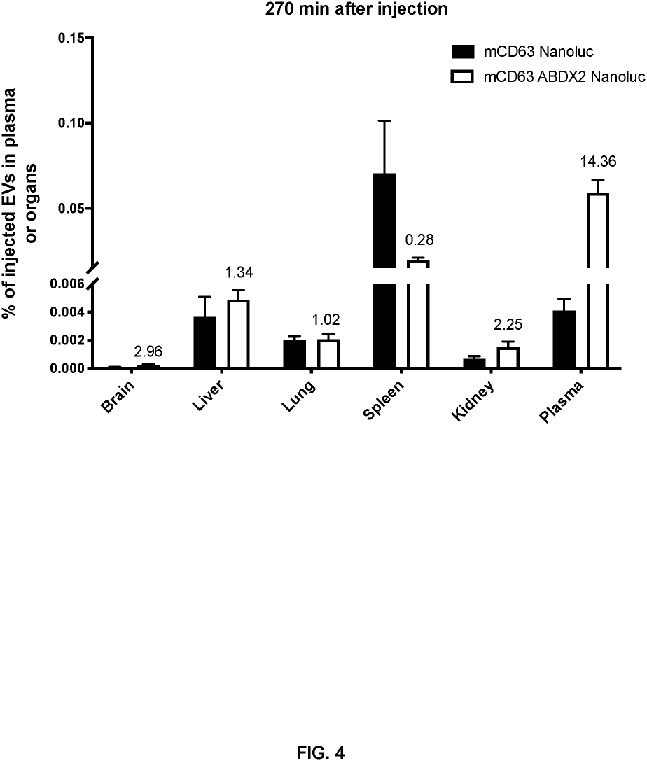

The data in FIG. 4 show that the presence of ABD on the surface of EVs increased the EV numbers in brain, kidney, plasma and liver. The numbers above the columns in FIG. 4 indicate the fold changes compared to the control group. A 3-fold increase was seen in the brain, a 2-fold increase was seen in the kidneys, a 14-fold increase was seen in plasma and a 1.3-fold increase was seen in liver. The presence of ABD on the surface of EVs did not affect EV biodistribution in the lung and decreased the EV biodistribution in spleen.

The presence of ABD is therefore beneficial for altering the biodistribution of EVs. This is especially true for EVs where the intended target organ is the brain or for the treatment of cancer, where high levels of EVs are desired in the plasma and lymph nodes. It is clear that the present invention would be extremely useful in the case of EVs which additionally comprise a targeting moiety; for instance, EVs which are engineered to target a particular organ or disease state, such as brain-targeted EVs or EVs comprising cancer targeting moieties.

### Example 5: Tumor Accumulation of ABD EVs

Following the discovery that the presence of ABD on the surface of EVs extends the half-life of the EVs in circulation and alters the biodistribution of EVs, it was predicted that improved half-life and altered biodistribution of EVs would impact on the tumor accumulation of those EVs. In addition, EVs may accumulate in tumor tissues by enhanced permeability and retention (EPR) effect because tumor vessels are permeable and tumor tissues lack functional lymphatic system. The EVs cannot circulate back to circulatory system. Furthermore, the tumor tissue normally expresses the receptors for albumin, and EVs binding to albumin will target to tumors in this way. In order to investigate the tumor accumulation of ABD EVs (mCD63-ABDx2-NanoLuc (SEQ ID NO:6)) compared to control EVs (mCD63-NanoLuc (SEQ ID NO:5)), C57 mice (3 mice per group) were injected with either control or albumin-binding EVs intravenously (1E11 EVs/ mouse). The ABD used in the constructs of Example 5 was ABD035 (SEQ ID NO: 4). 4.5 hours after injection, tumors were harvested and lysed in buffer (0.1% (v/v) TritonX-100) with tissue lyser. Nanoluc signals from the lysate were measured by luminometer.

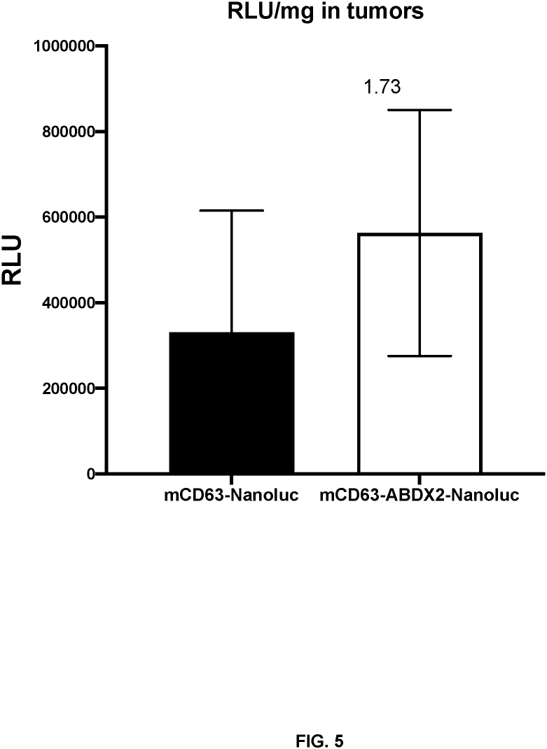

The data in FIG. 5 show that the presence of ABD on the surface of EVs increased the accumulation of EVs in tumors by 1.73-fold compared to the control group.

The presence of ABD is therefore beneficial for altering the biodistribution of EVs, enabling them to accumulate in tumors. This is especially useful in the treatment of cancers by EV therapeutics, which may entail loading the EV with a therapeutic cargo, such as a small oncostatic molecule, an anti-cancer antibody or an anti-cancer silencing RNA. Alternatively, or additionally, the therapeutic EV may comprise a neo-antigen, which is used to raise an immune response against the cancer by immunotherapy. Furthermore, the addition to the EV of a tumor targeting moiety would further increase the tumor accumulation of the ABD EVs.

### Example 6: ABD Increases Lymph Node Accumulation

Following the discovery that the presence of ABD on the surface of EVs extends the half-life of the EVs in circulation and alters the biodistribution of EVs, it was also predicted that improved half-life and altered biodistribution of EVs would impact on the accumulation of those EVs in lymph nodes. In addition, conjugation of ABD with nanoparticles has been shown to accumulate nanoparticles in lymph nodes. In order to investigate the lymph node accumulation of ABD EVs (mCD63-ABDx2-NanoLuc (SEQ ID NO:6)) compared to control EVs (mCD63-NanoLuc (SEQ ID NO:5)), NMRI mice (6 mice per group) were injected with either control or albumin-binding EVs intravenously (1E11 EVs/ mouse). The ABD used in the constructs of Example 6 was ABD035 (SEQ ID NO: 4). 4.5 hours after injection, lymph nodes were harvested and lysed in buffer (0.1% (v/v) TritonX-100) with tissue lyser. Nanoluc signals from the lysate were measured by luminometer.

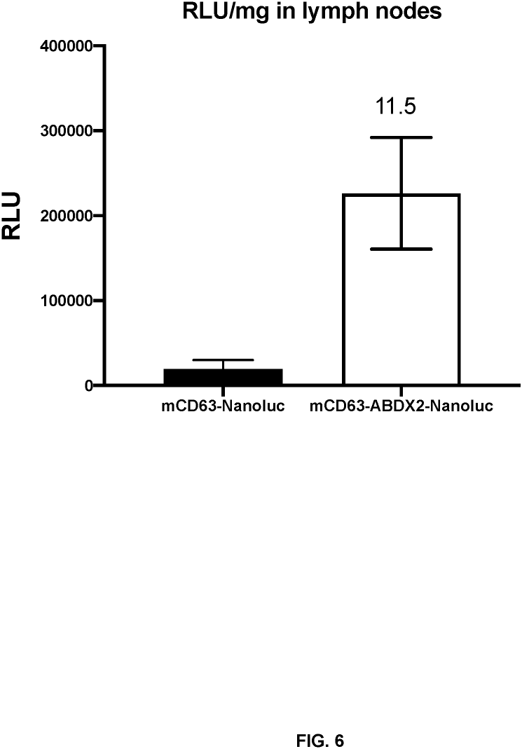

The data in FIG. 6 show that the presence of ABD on the surface of EVs drastically increased the accumulation of EVs in lymph nodes by 11.5-fold compared to the control group.

The presence of ABD is therefore beneficial for altering the biodistribution of EVs, enabling them to also accumulate in lymph nodes. This is especially useful when the therapeutic cargo is designed to elicit an immune response, i.e. an EV loaded with an antigen or multiple antigens designed to act as a vaccine, because T-cell-antigen presenting cell cross talk is known to occur in lymph nodes. Taking the data in FIGS. 5 and 6 together it can be seen that, in particular, ABD EVs would be especially useful in the treatment of cancers by immunotherapy, but treatment of any other condition by immunotherapy is also envisaged.

### Example 7: ABD-EV Albumin in Vitro Binding Assay

In order to verify the ABD present on the surface of EV is capable of binding albumin, an in vitro binding assay was performed using chromatography. The binding of fluorescein isothiocyanate (FITC)-labeled albumin to either ABD-EVs (mCD63-ABD 2^{nd} loop-NanoLuc (SEQ ID NO:8)) or control EVs lacking the ABD (mCD63-NanoLuc (SEQ ID NO:5)) plus a phosphate buffered saline (PBS) control.

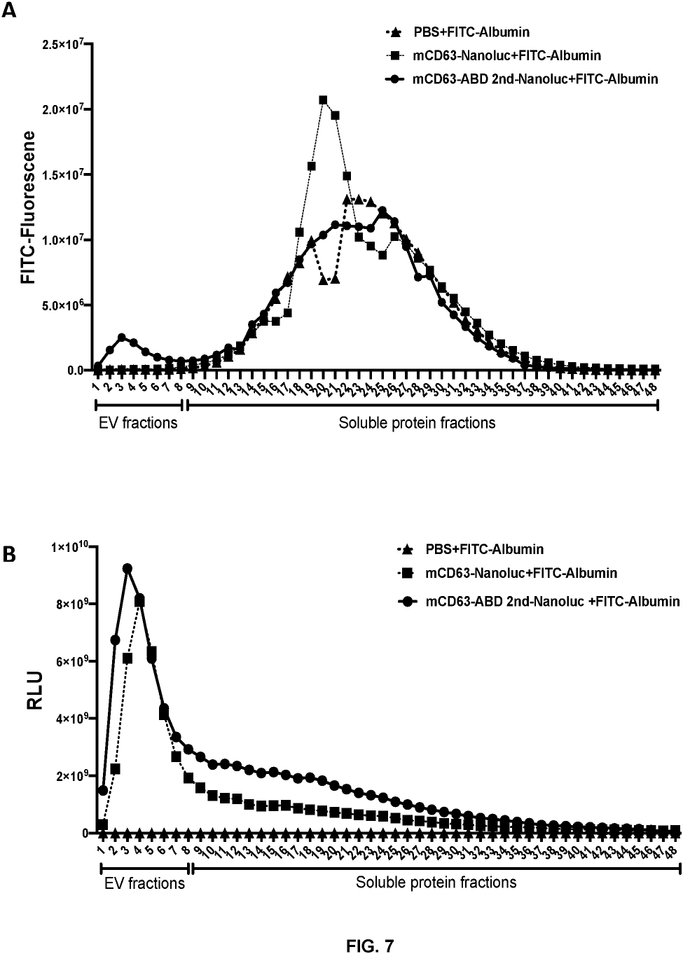

1E11 EVs were incubated with FITC-human albumin (360 µg/ml) at 37° C. for 2 h. Following this incubation, size exclusion chromatography was used to obtain 48 fractions for each sample, with fractions 1 to 8 as the EV fractions and 9 to 48 as soluble protein fractions. These data are presented in FIG. 7A.

The data in FIG. 7A for the EV fractions (1 to 8) show that only albumin-binding EVs (ABD-EVs) showed obvious FITC-fluorescence detected by SpectraMax, indicating the binding of EVs with FITC-albumin. Fractions 3 and 4 had the highest fluorescence signals.

FIG. 7B shows the nanoluc signals, which were tested in all 48 fractions to confirm that fractions 1 to 8 were EV fractions. Fractions 3 and 4 showed the highest nanoluc signals in the albumin-binding EV group (ABD-EVs), corresponding to the fluorescence signals detected in the FITC experiment.

### Example 8: ABD-EV Albumin Binding Assay by Flow Cytometry

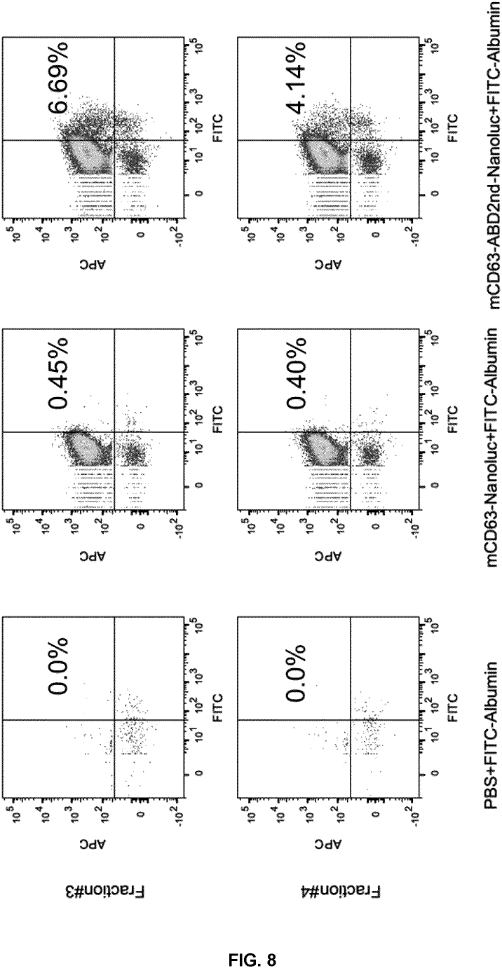

Following on from the chromatography data shown in FIG. 7, further in vitro analysis of the binding to albumin of ABD-EVs was undertaken. In order to verify that the ABD present on the surface of EVs is capable of binding albumin, fractions 3 and 4 from the chromatography experiment in Example 7 were taken for flow cytometry analysis by CellStream. A pan (CD9-CD63-CD81)-adenomatous polyposis coli (APC) antibody was used to stain the samples. The data in FIG. 8 show that only the albumin-binding EV group (ABD-EVs) showed double (APC and FITC) positive signals, with 6.69% and 4.14% in fractions 3 and 4, respectively.

It is important to note that, due to the need to dilute the samples during flow cytometry, this is likely to have caused dissociation of albumin from the EVs. Therefore the true percentage of double positive EVs is likely to be significantly higher than that presented in FIG. 8. Nevertheless, the data provided in FIG. 8 clearly show that albumin binds to engineered ABD-EVs. The data correlate well with the fluorescent signals detected by SpectraMax and nanoluc signals detected by luminometer.

### Example 9: Additional ABD-EV Albumin in Vitro Binding Assays

Following the success of the in vitro binding assay described in Example 7 and shown in FIGS. 7A and 7B. The same protocol as outlined in Example 7 was used but a wider range of constructs were tested. These constructs include a range of different EV proteins (human CD81, CD9 and CD63) and a range of different locations for the ABD (1^{st} loop, second loop or both loops(x2)).

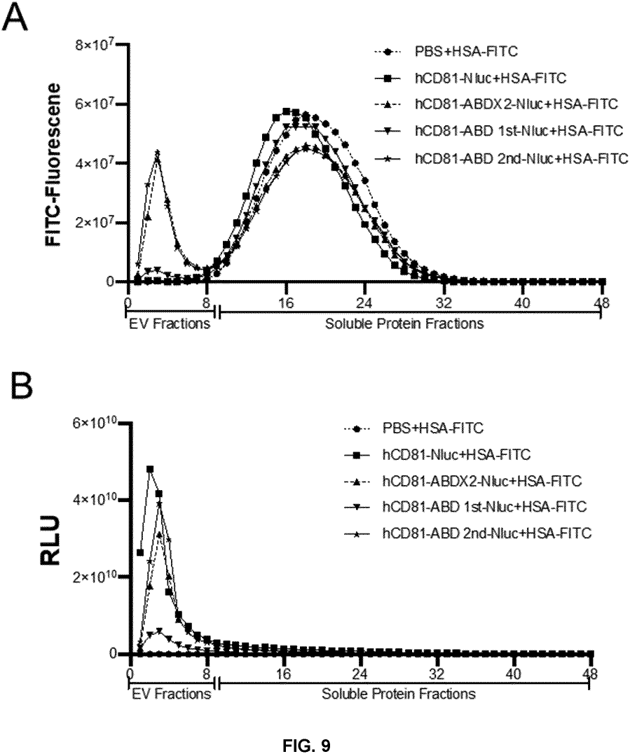

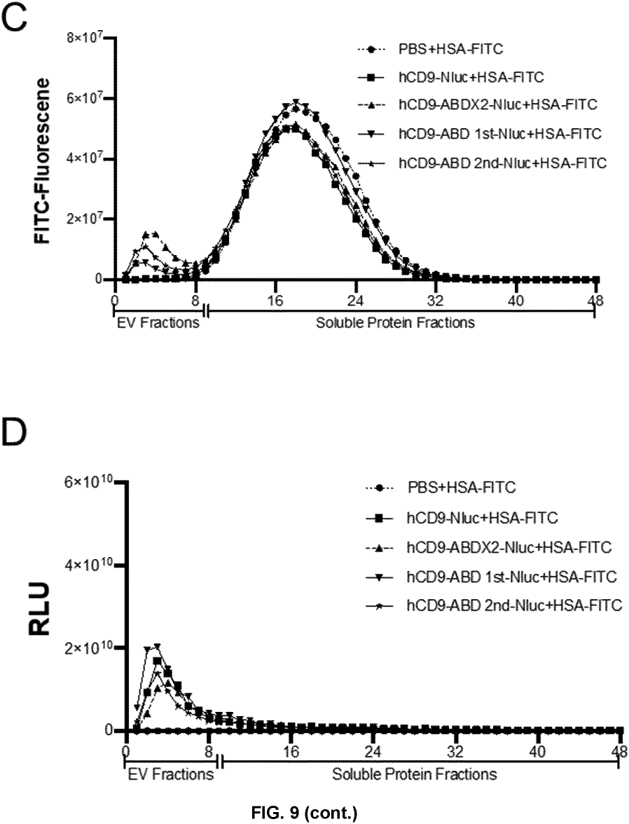

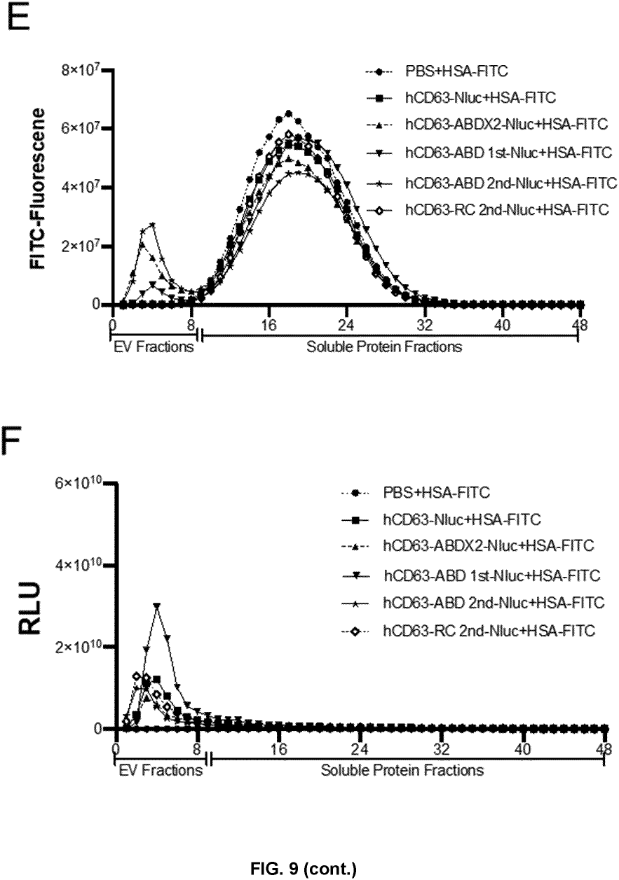

FIGS. 9 A-F show similar results to FIG. 7 indicating that FITC-albumin is bound on the surface of EVs when ABD is engineered into a fusion construct with a range of different EV proteins and at a range of different locations within the EV protein (1^{st} loop, second loop or both loops). This shows that the binding of albumin to the ABD is not dependent on the type of EV protein present in the fusion construct, nor the location of the ABD within the fusion protein.

Importantly, experiments where ABD is fused into both loops show that it is possible to engineer functional proteins into more than one loop of a multi-pass transmembrane EV protein without the EV protein being disrupted or affecting membrane insertion.

### Example 10: Additional in Vivo Half-Life Extension Experiments

Following the success of the experiments shown in Example 3 additional constructs were tested in vivo to assess the effect of ABD in combination with a range of different EV proteins and ABD located in different loops. The constructed tested were:
■ mouseCD81-ABD2nd loop
■ mouseCD9-ABD2nd loop
■ mouseCD63-ABD2nd loop
■ humanCD81-ABD2nd loop
■ humanCD9-ABD2nd loop
■ humanCD63-ABD2nd loop
■ mouseCD9-ABDx2 (both loops)
■ humanCD9-ABDx2 (both loops)

The protocol used is identical to that given in Example 3. In summary, NMRI mice, IV injection of 1E11 EVs and plasma collected after 4.5 h.

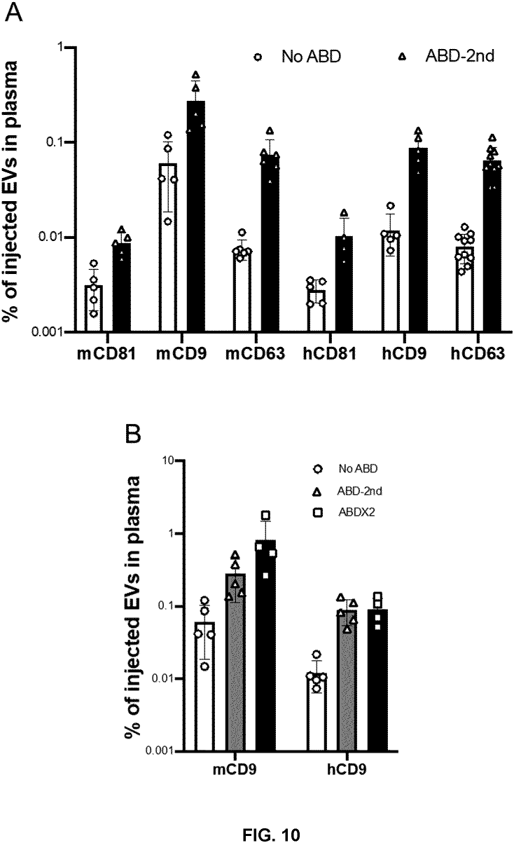

The data in FIGS. 10 A&B show that the presence of ABD significantly increases the number of EVs in circulation. The presence of ABD on the surface of EVs resulted in significant increases in the percentage of injected EVs in plasma across all constructs.

### Example 11: Additional in Vitro and in Vivo Half-life Extension Experiments using Single Pass Transmembrane Protein.

Experiments similar in design to those described in Examples 7 and 3 were performed to test the in vitro binding potential and in vivo half-life extension capability of fusion protein constructs utilizing a single pass transmembrane protein rather than a multi-pass transmembrane protein.

The protocol used is identical to that given in Examples 7 and 3. The constructs used the single-pass transmembrane EV protein Lamp2b:
Control: Lamp2b-NanoLuc
Active: Lamp2b-ABD-NanoLuc

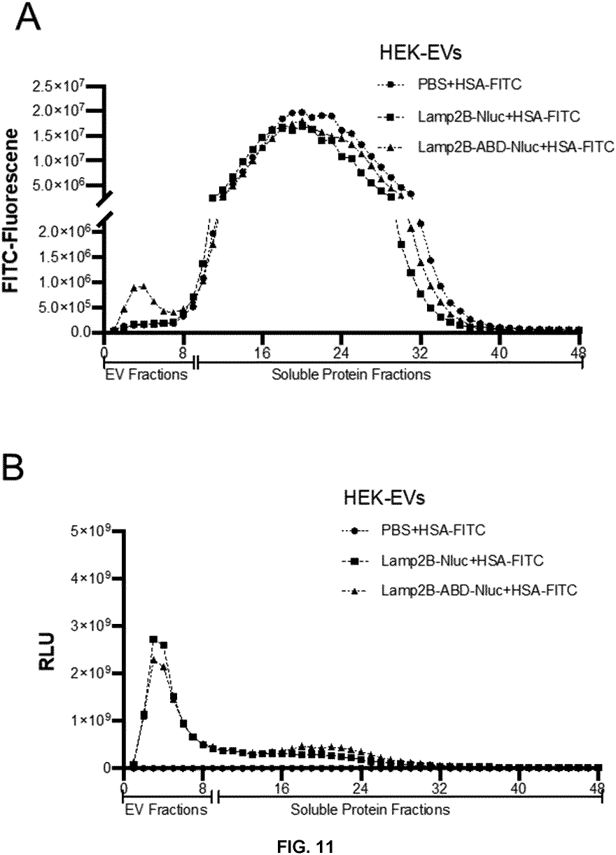

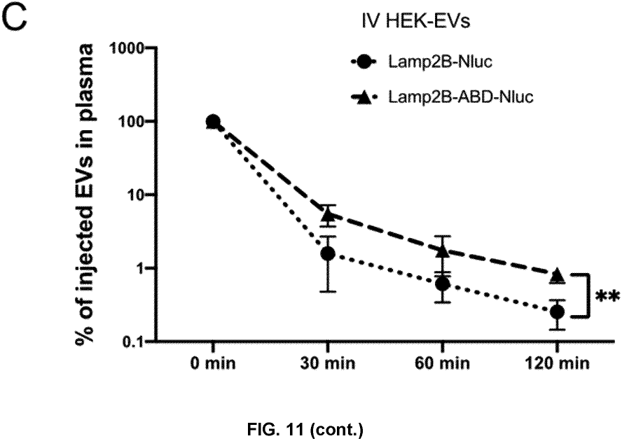

FIGS. 11 A&B shows engineering ABD into the single pass transmembrane protein Lamp2b causes binding of the ABD to the EVs (in in vitro assay) and FIG. 11C shows the same EVs, when in vivo, result in a significant increase in circulation time of 3-4 fold, indicating the ABD engineering is versatile platform to extend EVs in circulation using either single or multi-pass transmembrane proteins.

### Example 12: Additional Tumor/Lymph Node Accumulation Experiments

Following the evidence shown in Example 5 and Example 6 that ABD EVs accumulate in tumors and lymph nodes a similar additional experiment was performed to test tumor and lymph node accumulation using an alternative construct, specifically the CD63-ABD 2^{nd} loop construct.

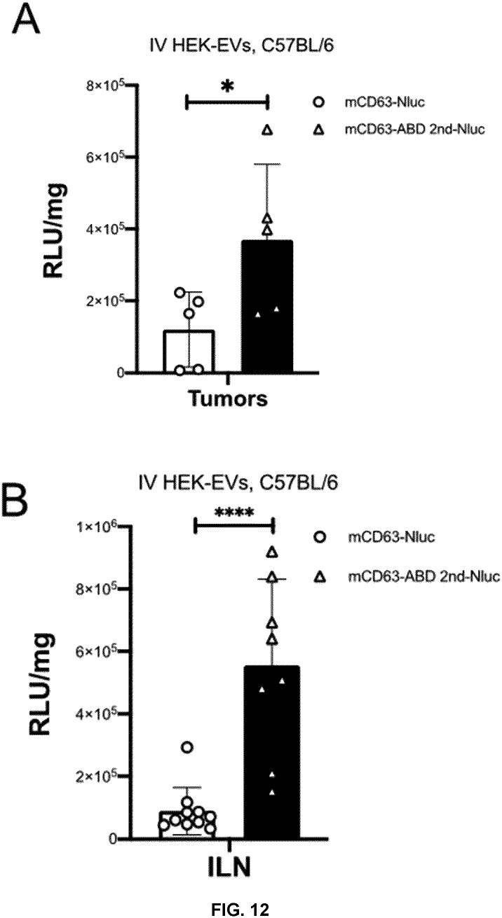

The data in FIG. 12 show that the presence of ABD on the surface of EVs significantly increased the accumulation of EVs in tumors (A) and lymph nodes (B) compared to the control group.

This data shows again that the presence of ABD is therefore beneficial for altering the biodistribution of EVs, enabling them to accumulate in tumors and lymph nodes. Importantly this data shows that the presence of ABD on the surface is broadly effective across different constructs, with different designs. As mentioned previously, the ability to target tumours is especially useful in the treatment of cancers by EV therapeutics, which may entail loading the EV with a therapeutic cargo, such as a small oncostatic molecule, an anti-cancer antibody or an anti-cancer silencing RNA. Alternatively, or additionally, the therapeutic EV may comprise a neo-antigen, which is used to raise an immune response against the cancer by immunotherapy. Furthermore, the addition to the EV of a tumor targeting moiety would further increase the tumor accumulation of the ABD EVs.

### Example 13: Half-Life in Plasma After Delivery by Different Routes of Administration.

In order to assess the potential of ABD EVs to extend half-life when administered via different routes of administration another experiment was performed. EVs expressing the fusion protein CD63-ABD-2ndloop-nanoLuc were compared to control EVs expressing only CD63-nanoluc via using the same in vivo protocol as described in Example 7 except the EVs were delivered by intravenous (IV), sub-cutaneous (SC) or intraperitoneal (IP) routes of administration.

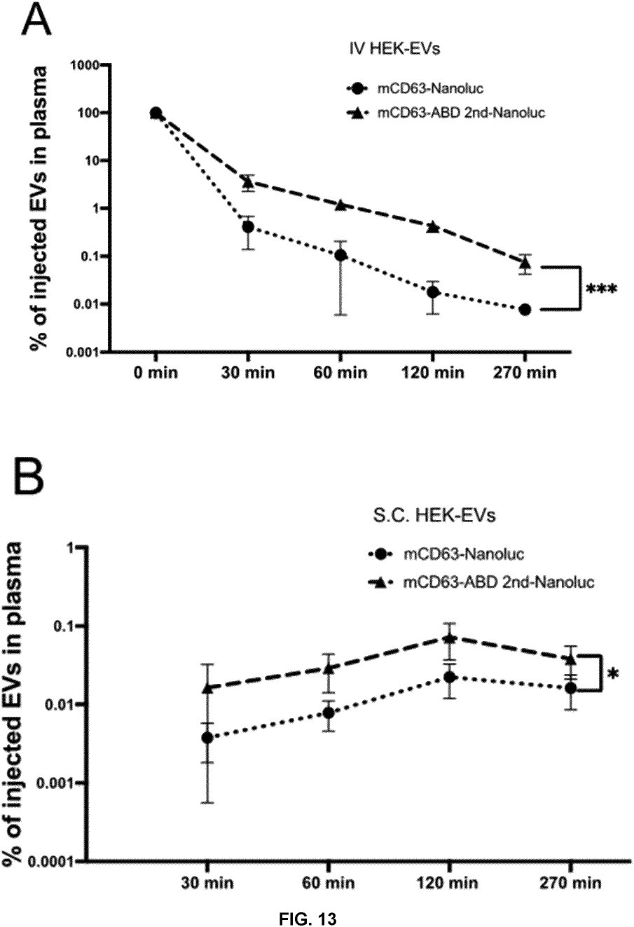

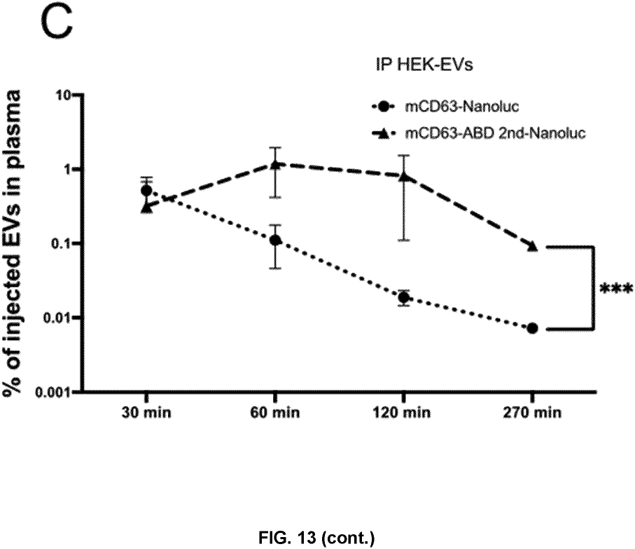

FIGS. 13 A-C shows that the presence of ABD increases the in vivo plasma levels of EVs compared to control regardless of the route of administration.

### Example 14: Half-Life in Plasma of EVs From Alternative Cell Source after IV Delivery.

Another in vivo half-life extension experiment similar to the protocol set out in Example 3 was conducted, this time with EVs derived from CAP cells (amniotic epithelial cells).

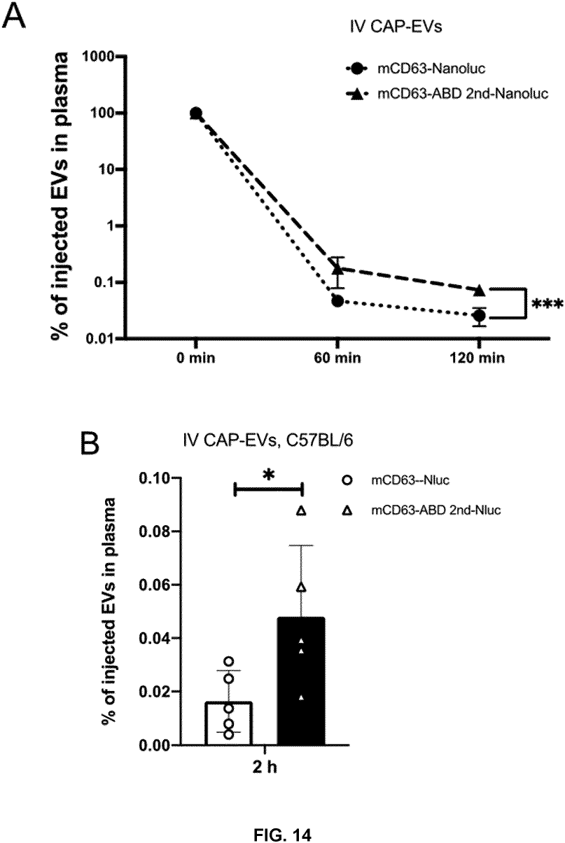

FIG. 14 shows the results from experiments testing CAP derived EVs expressing mouseCD63-ABD 2^{nd} loop. FIGS. 14 A and B show that similarly to data described above, EVs derived from CAP source cells which express ABD on the surface are present in the plasma at higher levels than control EVs showing that the ABD EVs have a longer half-life. This shows that the effect of engineering ABD into EVs is broadly applicable across EVs derived from different source cells.

Biodistribution experiments similar to those described in Example 4 were also performed for CAP derived EVs and demonstrated preferential accumulation in the brain, lymph nodes and tumors similar to the data shown for HEK derived EVs (data not shown).

### Example 15: Gradient Separation & Wide-Field Microscopy

Gradient separation was performed to assess the binding of albumin to EVs expressing ABD on the surface. The fusion constructs tested were:
mCD63-Nluc (concentration: 1.37×10¹² particles/ml)
mCD63-ABD-2nd loop Nluc (concentration: 6.5×10¹¹ particles/ml)

HEK EVs with albumin binding domain fused to different or both CD63 loops were incubated overnight at 4° C. 5 µl with 80 µg/ml human serum albumin (labelled with Alexa Fluor 488) and a mix of tetrspanin antibodies (CD9, CD63 and CD81, 1:100) labelled with Alexa Fluor 647. The next day, EVs were separated by Optiprep gradient ultracentrifugation and 300 µl fractions were collected, 100 µl from each fraction were used for DOTBLOT analysis and 50 µl from three subsequent fractions were pooled and used for imaging (widefield and dSTORM)

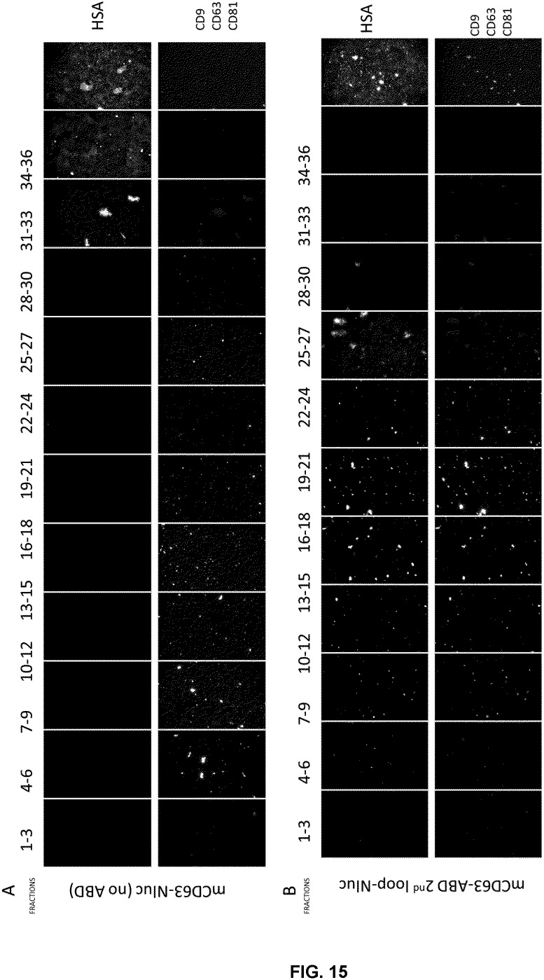

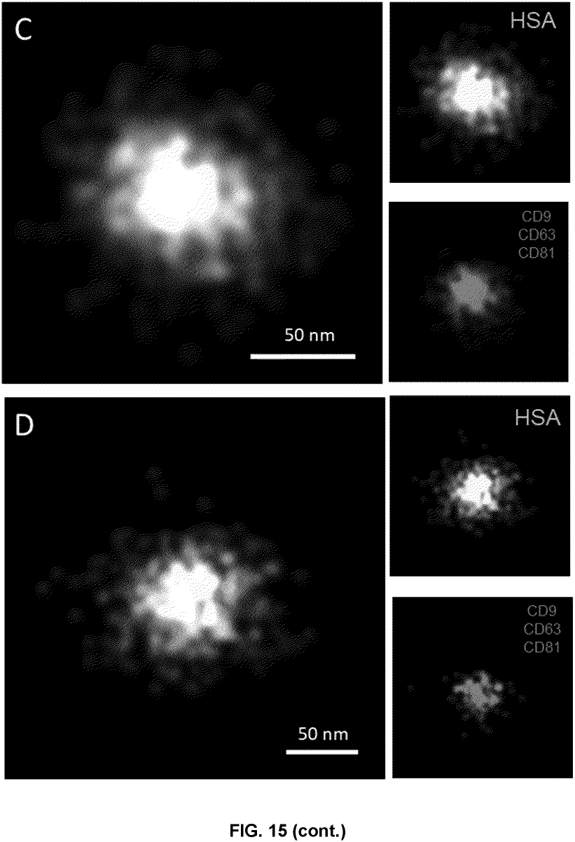

FIG. 15A shows images of the pooled fractions. This shows that when ABD is not present on the surface of EVs there is no overlap of HSA staining (top panel) with EV fractions (bottom panels stained with CD9/63/81) showing without ABD present HSA is not associated with Evs, HSA is only present in the aggregate fractions.

FIG. 15B shows that when ABD is present on the surface of EVs HSA staining (top panel) is observed to overlap with EVs (bottom panels stained with CD9/63/81) at fractions 10-24 showing that in the presence of ABD HSA is associated with EV fractions.

FIGS. 15C&D show by wide-field microscopy of the mCD63-ABD 2^{nd} loop-Nluc EVs from FIG. 15B showing that the HSA label overlaps with the EV markers CD9, CD63 and CD81. This again corroborates that the HSA is present on the surface to the ABD EVs.

### Example 16 - in Vivo for ABD Binding Assay by Dot-Blot

Following the confirmation that albumin is found bound to ABD EVs in vitro, in vivo experiments were then conducted to assess whether the presence of ABD on the surface of EVs results in albumin being bound after the EVs are injected into circulation and thus extends the half-life of those EVs.

Mice were injected with mCD63-ABD-2nd loop Nluc HEK EVs or mCD63-Nluc (no ABD control). Serum was collected 10 minutes after injection and purified with size-exclusion columns (qEV) to isolate EVs. 2.5×10¹⁰ EVs were then incubated overnight at 4° C. with an antibody anti-mouse serum albumin (labelled with FITC) and a mix of tetraspanin antibodies (human specific CD9, CD63 and CD81, 1:100) labelled with Alexa Fluor 647. The next day, EVs were separated by Optiprep gradient ultracentrifugation and 300 µl fractions were collected, 100 µl from each fraction were used for DOTBLOT analysis and 50 µl from three subsequent fractions were pulled and used for imaging (widefield)

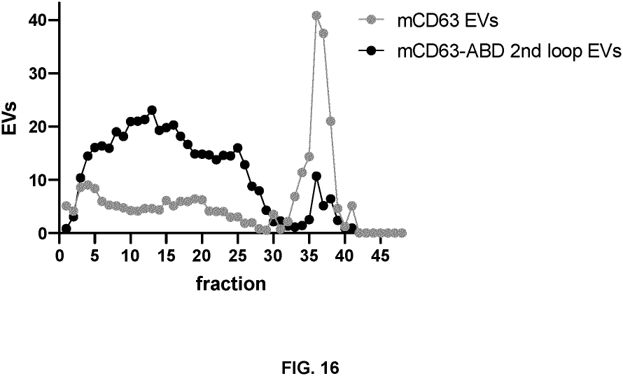

FIG. 16 shows the quantification of the DOTBLOT data. This graph shows that many more ABD EVs are retrieved from serum than control EVs showing again that ABD present on the surface, attracts albumin to the surface of the EVs and extends the plasma half-life of those EVs.

### Example 17 - in Vitro Binding Assay by Dot-Blot

Following on from Example 16 and the data in FIG. 16, further in vitro binding experiments were then performed to assess the binding of HSA to ABD when ABD is expressed on the surface of the EVs as part of different fusion constructs.

EVs comprising the following fusion constructs tested were:
hCD81-ABD-1 stloop-Nluc
hCD81-ABD-2ndloop-Nluc
hCD81-ABD-2x-Nluc (ABD present in both loops)
hCD81-ABD-Nluc
hCD9-ABD-1stloop-Nluc
hCD9-ABD-2ndloop-Nluc
hCD9-ABD-2x-Nluc (ABD present in both loops)
hCD9-ABD-Nluc
mCD63-ABD-2ndloop-Tluc
mCD63-Tluc
Lamp2b-ABD-Nluc
Lamp2b-Nluc

This experiment was performed by incubating overnight at 4° C. 4e¹⁰ HEK EVs expressing the constructs detailed above with 80 µg/ml human serum albumin (labelled with Alexa Fluor 488) and a mix of tetraspanin antibody. The next day, EVs were separated by Optiprep gradient ultracentrifugation and 300 µl fractions were collected, 100 µl from each fraction were used for DOTBLOT analysis and 50 µl from three subsequent fractions were pulled and used for imaging (widefield and dSTORM) dies (CD9, CD63 and CD81, 1:100) labelled with Alexa Fluor 64.

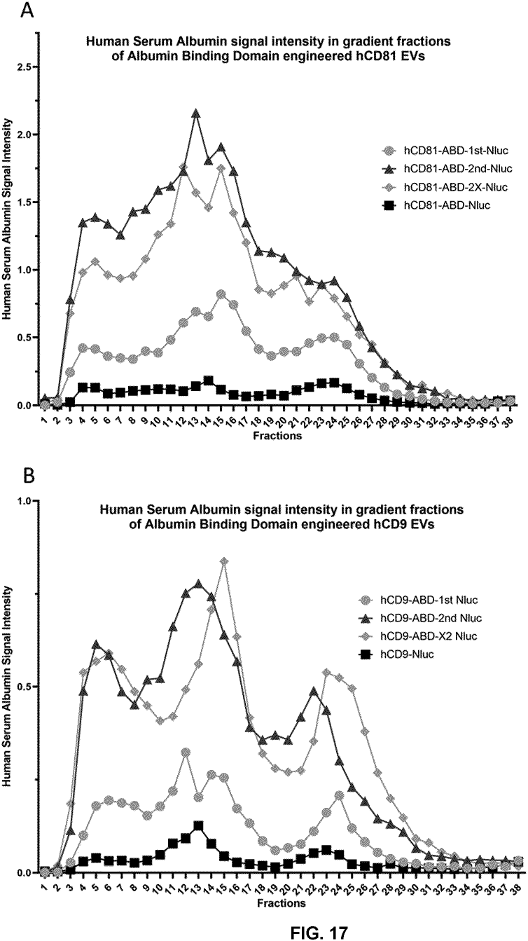

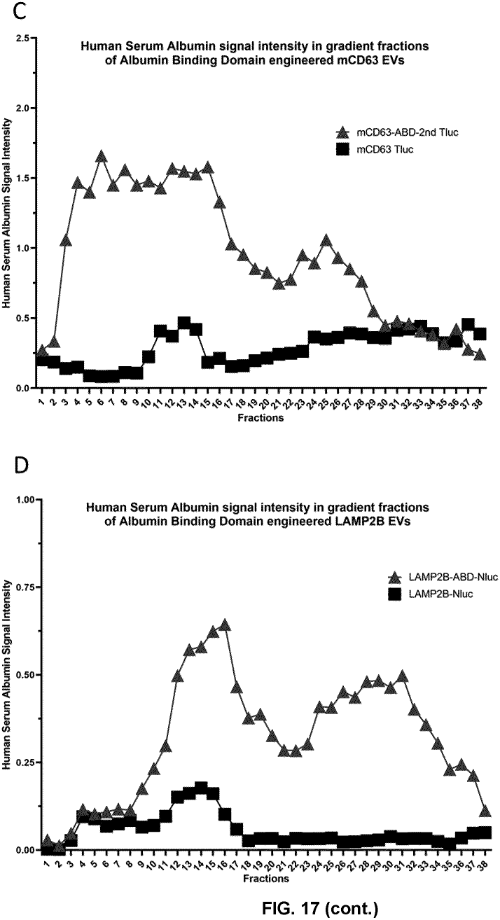

FIG. 17 A shows the quantification of the dot blots for the CD81 constructs.
FIG. 17 B shows the quantification of the dot blots for the CD9 constructs.
FIG. 17 C shows the quantification of the dot blots for the CD63 constructs.
FIG. 17 D shows the quantification of the dot blots for the Lamb2b constructs.

The data in FIG. 17 show that HSA levels are much increased in all samples where the ABD is present. This indicates that the presence of ABD on the surface of EVs enable the EVs to bind albumin and retain that albumin on the surface following processing indicating that the strength of albumin binding to ABD on the surface of EVs is relatively strong. Notably, this effect is independent of: a) the type of EV protein to which the ABD has been fused (multi-pass transmembrane or single pass transmembrane), or b) the location of the ABD (first, second or both loops of a tetraspanin).

### Example 18: ABD Increases Stability of EVs During Storage

As shown from the examples above, the presence of ABD on the surface of EVs can increase the stability, and thus half-life, of EVs in vivo. It is also therefore predicted that the presence of ABD on the surface of EVs can improve the stability of EVs in storage. The addition of albumin into the storage buffer for EVs would result in the EVs binding to the albumin and becoming coated in a protective shield of albumin. Such a protective shield is believed to protect the EVs against damage caused by freeze thaw cycles and thus improve the stability in formulation in addition to improving half-life in vivo. This stability is advantageous, as it will result in increased shelf-life of the pharmaceutical composition of the EVs, making a more robust versatile product that will remain bioactive for longer during storage.

ABD-EVs are obtained from an EV-producing genetically engineered and immortalized cell line cultured in bioreactors. CM containing the EVs is harvested from the bioreactors. EVs are then isolated from the CM by centrifugation, to remove cells and cell debris, and thereafter filtrated to remove any larger particles. The filtered CM is then run through a hollow fiber filter using a TFF system and concentrated down after diafiltration. The EVs are then combined with a formulation buffer comprising albumin and this formulation is then either a) stored for a period of time (at different temperatures (-80° C., -20° C., 4° C.) for up to 30 weeks); or b) subjected to repeated freeze thaw cycles.

The quality and robustness of the EV population is then tested following the prolonged periods of time in storage at different temperatures as well as the repeated freeze thaw cycles. EV number is tested using nanoparticle tracking analysis (NTA). The quality of the EVs is measured using a number of techniques:
i) by comparing the RNA content before and after storage/stress testing;
ii) EVs are tagged with a fluorescent label such as GFP or nanoluc and the levels of fluorescence/bioluminescence are analyzed before and after storage/stress testing by spectrometer (SpectraMax); and
iii) therapeutic functionality of EVs is tested before and after storage/stress testing using EVs loaded with a cargo, the effect of which can be observed in vivo. For instance, a splice switching cargo RNA may be added to the EVs. When those EVs are added to cells comprising a reporter that switches under the presence of the splice switching oligo, the effectiveness of the cargo loaded in the EVs is measurable. Alternatively, EVs expressing a decoy ligand on the surface, such as EVs genetically modified to display TNFalpha decoying receptors, may be tested in an NF-κB reporter cell model, which is genetically modified to express the NF-κB-luciferase reporter gene as a model for inflammation. The anti-inflammatory activity of the EVs comprising the TNFalpha decoying receptors can then be measured before and after storage/stress testing. In addition, ABD-EVs carrying Cre can also be used to test the function of Cre using a Cre-Lox reporter in recipient cells before and after storage/stress testing.

### Example 19: Purification of ABD EVs

It is also predicted that the binding affinity of ABD for albumin will allow the ABD EVs of the present invention to be purified using affinity chromatography. The steps of purification comprise: (i) contacting a medium comprising the ABD EVs with a chromatography matrix comprising albumin or a fragment or domain thereof, (ii) allowing the ABD EVs of the invention to adsorb to the albumin or fragment/domain thereof, and (iii) eluting the ABD EVs by passing across the chromatography matrix a medium that releases the ABD EVs from the albumin or fragment or domain thereof.

ABD EVs are obtained from CM collected from genetically engineered EV-producing cell lines grown in a hollow-fibre bioreactor. The secreted EVs comprise ABD present on the surface. The CM obtained from the bioreactor is loaded onto a column connected to a chromatography system. The chromatography matrix comprises albumin bound to the surface. Flow rate settings for column equilibration, sample loading and column cleaning in place procedure are chosen according to the manufacturer’s instructions. The medium comprising the ABD EVs is loaded onto the chromatography column and the ABD EVs bind to the matrix comprising albumin. An elution buffer is chosen to elute the ABD EVs from the column by altering the pH of the solution. The sample is then collected and stored at -80° C. for further downstream analysis, using flow cytometry, electron microscopy, and bioactivity assays.

Both the process of capturing the EVs and the process of releasing the EVs is repeated multiple times. The elution step comprises triggering the release of the EVs from the albumin or fragment/domain thereof by exposing the albumin - ABD bond to a medium with a suitable pH. This is achieved by running the EV-containing medium (i.e. the liquid phase) through a chromatography column comprising as stationary phase a chromatography matrix having attached to it albumin or fragment/domain thereof, letting the ABD polypeptides of the EVs adsorb to the albumin or fragment/domain thereof present on the matrix, and then running a solution with a suitable pH through the chromatography column. The pH of the solution intended to trigger release of the EVs from the column may be below pH 8, below pH 7, or below pH 6.

### SEQUENCE LISTING

### Amino Acid Sequences of ABDs

SEQ ID NO:1 ABD001(WT)

SDYYKNLINNAKTVEGVKALIDEILAALP

SEQ ID NO:2 ABD011

SDYYKNIINRAKTVEGVRALKLHILAALP

SEQ ID NO:3 ABD013

SDYYKNLINKAKTVEGVEALTLHILAALP

SEQ ID NO:4 ABD035

SDFYKRLINKAKTVEGVEALKLHILAALP

### Amino Acid Sequences of Constructs Used with His Tag (Optional). Nucleotide Sequence Identifiers Encoding Constructs used are also Provided in Parentheses and in the Sequence Listing, which is Incorporated by Reference in its Intirety.

SEQ ID NO:5 mCD63-Nanoluc (SEQ ID NOs: 34 and 44)

MAVEGGMKCVKFLLYVLLLAFCACAVGLIAIGVAVQVVLKQAMHITHETT
AGSLLPVVIIAVGAFLFLVAFVGCCGACKENYCLMITFAIFLSLIMLVEV
AVAIAGYVFRDQVKSEFNKSFQQQMQNYLKDNKTATILDKLQKENNCCGA
TRSNYTDWENIPGMAKDRVPDSCCINITVGCGNDFKESTIHTQGCVETIA
IWLRKNILLVAAAALGIAFVEVLGIIFSCCLVKSIRSGYEVMGSGSGSGS
GSVFTLEDFVGDWRQTAGYNLDQVLEQGGVSSLFQNLGVSVTPIQRIVLS
GENGLKIDIHVIIPYEGLSGDQMGQIEKIFKVVYPVDDHHFKVILHYGTL
VIDGVTPNMIDYFGRPYEGIAVFDGKKITVTGTLWNGNKIIDERLINPDG
SLLFRVTINGVTGWRLCERILAHHHHHH*

SEQ ID NO:6 mCD63-ABDX2-Nanoluc (SEQ ID NOs: 35 and 45)

MAVEGGMKCVKFLLYVLLLAFCACAVGLIAIGVAVQWLKQAMHGSLAEAK
VLANRELDKYGVSDFYKRLINKAKTVEGVEALKLHILAALPGSELMHITH
ETTAGSLLPVVIIAVGAFLFLVAFVGCCGACKENYCLMITFAIFLSLIML
VEVAVAIAGYVFRDQVKSEFNKSFQQQMQNYLKDNKTATILDKLQKENNC
CGATRRTQHDEAVDANSLAEAKVLANRELDKYGVSDFYKRLINKAKTVEG
VEALKLHILAALPRTCTSGTRSNYTDWENIPGMAKDRVPDSCCINITVGC
GNDFKESTIHTQGCVETIAIWLRKNILLVAAAALGIAFVEVLGIIFSCCL
VKSIRSGYEVMGSGSGSGSGSVFTLEDFVGDWRQTAGYNLDQVLEQGGVS
SLFQNLGVSVTPIQRIVLSGENGLKIDIHVIIPYEGLSGDQMGQIEKIFK
VVYPVDDHHFKVILHYGTLVIDGVTPNMIDYFGRPYEGIAVFDGKKITVT
GTLWNGNKIIDERLINPDGSLLFRVTINGVTGWRLCERILAHHHHHH*

SEQ ID NO:7 mCD63-ABD 1st Loop-Nanoluc (SEQ ID NOs: 36 and 46)

MAVEGGMKCVKFLLYVLLLAFCACAVGLIAIGVAVQWLKQAMHGSLAEAK
VLANRELDKYGVSDFYKRLINKAKTVEGVEALKLHILAALPGSELMHITH
ETTAGSLLPVVIIAVGAFLFLVAFVGCCGACKENYCLMITFAIFLSLIML
VEVAVAIAGYVFRDQVKSEFNKSFQQQMQNYLKDNKTATILDKLQKENNC
CGATRSNYTDWENIPGMAKDRVPDSCCINITVGCGNDFKESTIHTQGCVE
TIAIWLRKNILLVAAAALGIAFVEVLGIIFSCCLVKSIRSGYEVMGSGSG
SGSGSVFTLEDFVGDWRQTAGYNLDQVLEQGGVSSLFQNLGVSVTPIQRI
VLSGENGLKIDIHVIIPYEGLSGDQMGQIEKIFKVVYPVDDHHFKVILHY
GTLVIDGVTPNMIDYFGRPYEGIAVFDGKKITVTGTLWNGNKIIDERLIN
PDGSLLFRVTINGVTGWRLCERILAHHHHHH*

SEQ ID NO:8 mCD63-ABD 2nd Loop-Nanoluc (SEQ ID NOs: 37 and 47)

MAVEGGMKCVKFLLYVLLLAFCACAVGLIAIGVAVQVVLKQAMHITHETT
AGSLLPVVIIAVGAFLFLVAFVGCCGACKENYCLMITFAIFLSLIMLVEV
AVAIAGYVFRDQVKSEFNKSFQQQMQNYLKDNKTATILDKLQKENNCCGA
TRRTQHDEAVDANSLAEAKVLANRELDKYGVSDFYKRLINKAKTVEGVEA
LKLHILAALPRTCTSGTRSNYTDWENIPGMAKDRVPDSCCINITVGCGND
FKESTIHTQGCVETIAIWLRKNILLVAAAALGIAFVEVLGIIFSCCLVKS
IRSGYEVMGSGSGSGSGSVFTLEDFVGDWRQTAGYNLDQVLEQGGVSSLF
QNLGVSVTPIQRIVLSGENGLKIDIHVIIPYEGLSGDQMGQIEKIFKVVY
PVDDHHFKVILHYGTLVIDGVTPNMIDYFGRPYEGIAVFDGKKITVTGTL
WNGNKIIDERLINPDGSLLFRVTINGVTGWRLCERILAHHHHHH*

SEQ ID NO:9 mCD63-ABDX2-Neo-Nanoluc (SEQ ID NO: 38)

MAVEGGMKCVKFLLYVLLLAFCACAVGLIAIGVAVQWLKQAMHGSLAEAK
VLANRELDKYGVSDFYKRLINKAKTVEGVEALKLHILAALPGSELREGVE
LCPGNKYEMRRHGTTHSLVIHDDSGSPFPAAVILRDALHMARGLKYLHQE
LMHITHETTAGSLLPVVIIAVGAFLFLVAFVGCCGACKENYCLMITFAIF
LSLIMLVEVAVAIAGYVFRDQVKSEFNKSFQQQMQNYLKDNKTATILDKL
QKENNCCGATRRTQHDEAVDANSLAEAKVLANRELDKYGVSDFYKRLINK
AKTVEGVEALKLHILAALPRTCTSGVYDFFVWLGRGHLLGRLAAIVGKQV
LLGRKVVVVRSHCHWNDLAVIPAGVVHNWDFEPRKVSCTPSKPSFQEFVD
WENVSPELNSTDQPFLSGTRSNYTDWENIPGMAKDRVPDSCCINITVGCG
NDFKESTIHTQGCVETIAIWLRKNILLVAAAALGIAFVEVLGIIFSCCLV
KSIRSGYEVMGSGSGSGSGSVFTLEDFVGDWRQTAGYNLDQVLEQGGVSS
LFQNLGVSVTPIQRIVLSGENGLKIDIHVIIPYEGLSGDQMGQIEKIFKW
YPVDDHHFKVILHYGTLVIDGVTPNMIDYFGRPYEGIAVFDGKKITVTGT
LWNGNKIIDERLINPDGSLLFRVTINGVTGWRLCERILAHHHHHH*

SEQ ID NO:10 mCD63-ABDX2-Mut30-Nanoluc (SEQ ID NO: 39)

MAVEGGMKCVKFLLYVLLLAFCACAVGLIAIGVAVQWLKQAMHGSLAEAK
VLANRELDKYGVSDFYKRLINKAKTVEGVEALKLHILAALPGSELMHITH
ETTAGSLLPVVIIAVGAFLFLVAFVGCCGACKENYCLMITFAIFLSLIML
VEVAVAIAGYVFRDQVKSEFNKSFQQQMQNYLKDNKTATILDKLQKENNC
CGATRRTQHDEAVDANSLAEAKVLANRELDKYGVSDFYKRLINKAKTVEG
VEALKLHILAALPRTCTPSKPSFQEFVDWENVSPELNSTDQPFLSGTRSN
YTDWENIPGMAKDRVPDSCCINITVGCGNDFKESTIHTQGCVETIAIWLR
KNILLVAAAALGIAFVEVLGIIFSCCLVKSIRSGYEVMGSGSGSGSGSVF
TLEDFVGDWRQTAGYNLDQVLEQGGVSSLFQNLGVSVTPIQRIVLSGENG
LKIDIHVIIPYEGLSGDQMGQIEKIFKVVYPVDDHHFKVILHYGTLVIDG
VTPNMIDYFGRPYEGIAVFDGKKITVTGTLWNGNKIIDERLINPDGSLLF
RVTINGVTGWRLCERILAHHHHHH*

SEQ ID NO:11 mCD9-Nluc (SEQ ID NO: 40)

PSYNJJVNFLN\OOIJIQILIZ ODJLDYODLJOZ OUIGVTWNVPKLIHTHQQKVVI\WJY\LOLDJDO
PPOYJIOJFFJDYTHVTFPOJOIIJIOOYIIDIHIDDDYZ J \WKNGHYINHOTHI\NGW\TNOUVNGH
STUHWONDIKPDOGFFJLWUDJUDJSOHTIIVGWFSNNTOOHVITYNSFSHDIVHYIQQNIKIMDYJIJIDYY
PLIJPIIVPIOFFDHJUVUHPYJVYIWOHGIYJGZ UTWDJ\QOGTYOHTJJYVVOITQOJYVYWSIT
UIYOVJHQJONIGIKYIIS\HJOVJGTPJTIHNIINYY\SYGGKKINYIOK\JWOYIGJYWSQPLG\IJUS
\HJIDYIGJNNIWYWJWOZ QJQNILGHUOIQSGJVOOIUYWIQJYWJZ UOFHUIOD#

SEQ ID NO:12 mCD9-ABDX2-Nluc (SEQ ID NO: 41)

PSYNJJVNFIN\OOIJIQILIZ ODJLDYODLJOZ OUIGVTWNVPKODHDNYODQUHOGN\JYVGI\NU
OIQNDNWYHJYHDONOKIODDOSPKIIHTHQQKVVI\WJY\IOLJDJDOPPOYJIOJFFJDYTHVTFPO
JOIIJ IOOYIIDIHIDDDYZ J \ WKNGHYINHOT HI\NGW\TNOUVNGHSTUHWONDIKPDOGFFJIWUTK
GHDYGDQVODHDNYODQUHOGN\JYVGI\NUOIQNDNWYHJYHDONOKIODDOSWUDJSOHTIIVGWFS
NNT OOHVIT YNSF SHDIVHYIQQNIKLLJ DYJ LJ LLDYYP LLJP LLVP LOF FDLUUVUHP YJ VY IWOHG IYJ
GZ UTWDJ \QOGTYOHTJJYVVOITQOJYVYWSITUIYOVJHQJONLGLKYHS\HJOVJGTPJTLHNLI
NYY\SYGGKKINYLOK\JWOYLGJYWSQPLG\IJUS\HJLDYIGJNNLWYWJWOZ QJQNLLGHUOLQSGJ
VOOIUYWLQJYWJZ UOFHUIOD#

SEQ ID NO: 13 mCD9-ABD 1 st-Nluc (SEQ ID NO: 42)

P SYNJJVNFLN\OOIJIQILIZ ODJITJYODLJOZ OUIGVTWNVP KODHDNYODQUHOGN\JYVGI\NU
OIQNDNWYYHJYHDONOKIODDOSPKLIHTHQQKVVI\WJY\LOLJDJDOPPOYJIOJFFJDYTHVTFPO
JOIIJ IOOYIIDIHIDDDYZ J\WKNGHYLNHOTHI\NGW\TNOUVNGHSTUHWONDIKPDOGFFJLWUDJ
SOHT ILVGWF SNNT OOHVIT YNSF SHDLVHYIQQNIKLLJDYJLJLDYYP LIJP LIVP LOFFDLUUVUHP Y
J VYIWOHG IYJ GZ UT WDJ \ QOGTYOHTJJYVVOITQOJYVYWSLTULYOVJHQJONLGLKYLLS\HJOV
JGTPJTLHNUNYY\SYGGKKINYLOK\JWOYLGJYWSQPLG\IJUS\HJLDYIGJNNIWYWJWOZ QJQ
NLLGHUOLQSGJVOOIUYWLQJYWJZ UOFHUIOD#

SEQ ID NO:14 mCD9-ABD 2 nd-Nluc (SEQ ID NO: 43)

P SYNJJVNFLN\OOIJIQILIZ ODJITJYODLJOZ OUIGVTWNVPKLIHTHQQKVVI\WJY\LOLJDJDO
PPOYJIOJFFJDYTHVTFPOJOIIJIOOYLIDLHIDDDYZ J \ WKNGHYINHOTHI\NGW\TNOUVNGH
STUHWONDIKP DOGFFJLWUT KGHDYGDQVODHDNYODQUHOGN\JYVG I\NUOIQNDNWYHJYHDON
OKLODDOSWUDJSOHTILVGWFSNNTOOHVITYNSFSHDLVHYIQQNIKLLJDYJLJIDYYPLIJPLIVPLO
FFDLUUVUHPYJVYIWOHGIYJGZ UTWDJ\QOGTYOHTJJYVVOITQOJYVYWSLTULYOVJHQJON
LGLKYLLS\HJOVJGTPJTLHNLINYY\SYGGKKINYIOK\JWOYIGJYWSQPLG\IJUS\HJLDYIGJNN
IWYWJWOZ QJQNILGHUOLQSGJVOOIUYWLQJYWJZ UOFHULOD#

SEQ ID NO:15 mCD81-Nluc (SEQ ID NO: 48)

PJYHJFWNFLN\OOIYIQIYIZ ODJJYLOJYDOZ OUKGSTWWVOO\PKOHOJQNSDSQWI\YJL\LOLD
YJDYP P IYJ IOJF\JDLTHVTFOOJWIIWFOYIDIDFHYDDJLZ J IYQNGTILDNGYNT I\GTDOTTD
YPGGGDQQDNDYYNWIKHWOQFFJVWUQDOWWOWWWIDUQVOFSVJJQLOWSOOTTGFKTNLGHOIVJ
NO\OLJLDDLYYDYLPLIHPLOVPYOFFJLUQVVY\JVYIWOHGIYJGZ UTWDJ \QOGTYOHTJJYVVOI
TQOJYVYWSLTULYOVJHQJONIGLKYUS\HJOVJGTPJTIHNLINYY\SYGGKKINYLOK\JWOYLGJY
WSQPLG \IJUS\HJLDYIGJNNLWYWJWOZ QJQNLLGHUOLQSGJVOOIUYWLQJYWJZ UOFHUIOD#

SEQ ID NO:16 mCD81-ABDX2-Nluc (SEQ ID NO: 49)

P JYHJFWNFLN\OOIYIQIYIZ ODJJYIOJYDOZ OUKGSTWWVOO\ P KODHDNYODQUHOGN\JYVG
I\NUOIQNDNWYHJYHDONOKIODDOSPKOHOJQNSDSQWI\YJL\LOLDYJDYPP IYJIOJF\JDLTHV
TFOOJWIIWFOYLOIDFHYDDJLZ J IYQNGTILDNGYNT I\GTDOTTDYP GGGDQQDNDYYNWIKHWO
QFFJVWUTKGHDYGDQVODHDNYODQUHOGN\JYVGI\NUOLQNDNWYHJYHDONOKLODDOSWUQDO
WWOWWWLOUQVOFSVJJ QLOWSOOT TGFKTNLGHOIVJNO\OLJ ILDDIYYDYIP LIHP LOVP YOF FJLUQVV
Y\JVYIWOHGIYJGZ UTWDJ\QOGTYOHTJJYVVOITQOJYVYWSLTULYOVJHQJONLGIKYLLS\HJ
OVJGTPJTLHNLINYY\SYGGKKINYLOK\JWOYLGJYWSQPLG\IJUS\HJLDYIGJNNIWYWJWOZ Q
JQNUGHUOIQSGJVOOIUYWIQJYWJZ UOFHUIDD#

SEQ ID NO:17 mCD81-ABD 1 st-Nluc (SEQ ID NO: 50)

P JYHJFWNFLN\OOIYIQIYIZ ODJJYIOJYDOZ OUKGSTWWVOO\ P KODHDNYODQUHOGN\JYVG
I\NUOLQNDNWYHJYHDONOKIDDDOSP KOHOJQNSDSQWI\YJL\LOILDYJDYP P IYJIOJF\JDLTHV
TFOOJWIIWFOYIOIDFHYDDJIZ J IYQNGTILDNGYNT I \ GTDOTTDYP GGGDQQDNDYYNWIKHWO
QFFJVWUQDOWWOWWWIDUQVOFSVJJQLOWSOOTTGFKTNIGHOIVJNO\OLJLDDLYYDYIPLIHPLOVP
YOFFJLUQVVY\JVYIWOHGIYJGZ UTWDJ \QOGTYOHTJJYVVOITQOJYVYWSLTULYOVJHQJO
NLGLKYLLS\HJOVJGTPJTLHNLINYY\SYGGKKINYLOK\JWOYLGJYWSQPLG\ IJUS\HJLDYIGJN
NIWYWJWOZ QJQNILGHUOLQSGJVOOIUYWLQJYWJZ UOFHUIOD#

SEQ ID NO:18 mCD81-ABD 2 nd-Nluc (SEQ ID NO: 51)

PJYHJFWNFLN\OOIYIQIYIZ ODJJYLOJYDOZ OUKGSTWWVOO\ P KOHOJ QNSDSQWI \YJL\LOID
YJDYP P IYJ IOJF\JDLTHVTFOOJWIIWFOYIDIDFHYDDJLZ J IYQNGTILDNGYNT I\GTDOTTD
YPGGGDQQDNDYYNWIKHWOQFFJVWUTKGHDYGDQVODHDNYODQUHOGN\J YVGI\NUOIQNDNW
YHJ YHDONOKLODDOSWUQDOWWOWWWLOUQVOF SVJ J QLOWSOOTTGF KTNLGHOIVJ NO\ OLJ IDDIYYD
YLP LIHP LOVP YOFF J LUQVVY\JVYIWOHGIYJGZ UTWDJ \QOGTYOHTJJYVVOITQOJYVYWSLT
ULYOVJHQJONLGLKYLLS \HJ OVJGTPJTLHNLINYY\SYGGKKINYLOK\JWOYLGJYWSQPLG \ IJUS
\HJLDYIGJNNLWYWJWOZ QJQNILGHUOIQSGJVOOIUYWIQJYWJZ UOFHUIOD#

SEQ ID NO:19 hCD9-Nluc (SEQ ID NO: 52)

P SYNJ JWNFIN\OOIJ IQIIIZ ODJITJYODLJOZ OUIGVT WNVIP K IHTHWQQQQVVI\ WJY\LOLJDJD
OP P OYJ IOJFFJDYTHVTFP OJOIIJ IOOYIIDIHIDDDIZ J\VKNGHYLNHYTHI\NGW\QNONWNGH
STUHWONDLK\DOQFFJOWUDJJYHTIIVGLFSNNGYOHWIWYNVFSGDLNHYIGQNIKHJDYJULDYY
PLIJPLIVPLOFFDLUUQUHPYJVYIWOHGIYJGZ UTWDJ\QOGTYOHTJJYVVOITQOJYVYWSLT
ULYOVJHQJONLGLKYLLS\HJOVJGTPJTLHNLINYY\SYGGKKINYLOK\JWOYEGJYWSQPLG\IJUS
\HJLDYIGJNNIWYWJWOZ QJQNILGHUOIQSGJVOOIUYWIQJYWJZ UOFHUIOD#

SEQ ID NO:20 hCD9-ABDX2-Nluc (SEQ ID NO: 53)

P SYNJ JWNFIN\OOIJ IQIIIZ ODJITJYODLJOZ OUIGVTWNVI
P KODHDNYODQUHOGN\JYVGI\NUOLQNDNWYHJYHDONOKLODDOSP
KIHTHWQQQQVVI\WJY\LOLDJDOPPOYJIOJFFJDYTHVTFP OJOII
J IOOYIIDIHIDDDIZ J \VKNGHYLNHYTHI\NGW\QNONWNGHSTU
HWONDLK\DOQFFJOWUT KGHDYGDQVODHDNYODQUHOGN\JYVGI\N
UOLQNDNWYHJYHDONOKLODDOSWUDJJYHTILVGLFSNNGYOHWIWYN
VFSGGDLNHYIGQNIKILLJDYJLJYYPLIJPLIVPLOFFDLUUQUHPYJ
VYIWOHGIYJGZ UTWDJ \QOGTYOHTJJYVVOITQOJYVYWSLTULYO
VJHQJONLGLKYLLS\HJOVJGTPJTLHNLINYY\SYGGKKINYLOK\JW
OYLGJYWSQPLG\IJUS\HJLDYIGJNNIWYWJWOZ QJQNLLGHUOLQS
GJVOOIUYWLQJYWJZ UOFHUIOD#

SEQ ID NO:21 hCD9-ABD 1 st-Nluc (SEQ ID NO: 54)

P SYNJJWNFLN\OOIJIQILIZ ODJITJYODLJOZ OUIGVTWNVIPK
ODHDNYODQUHOGN\JYVGI\NUOIQNDNWYHJYHDONOKIODDOSPKIH
THWQQQQVVI\WJY\LOLJDJDOPPOYJIOJFFJDYTHVTFP OJOIIJ
IOOYIIDIHIDDDIZ J \VKNGHYLNHYTHI\NGW\QNONWNGHSTUHW
ONDLK\DOQFFJOWUDJJYHT ILVGLFSNNGYOHWIWYNVFSGDLNHYI
GQNIKLLJDYJLJLDYYP LIJP IIVP LOFFDLUUQUHP YJVYIWOH
GIYJGZ UTWDJ \Q OGTYOHTJJYVVOITQOJYVYWSLTULYOVJHQJ
ONLGLKYLLS\HJOVJGTP JTLHNLINYY\SYGGKKINYLOK\JWOYIG
JYWSQP IG\IJUS\HJLDYIGJNNLWYWJWOZ QJ QNLLGHUOLQSGJ
VOOIUYWLQJYWJZ UOFHUIOD#

SEQ ID NO:22 hCD9-ABD 2 nd-Nluc (SEQ ID NO: 55)

P SYNJJWNFLN\OOIJIQILIZ ODJITJYODLJOZ OUIGVTWNVIPKIHTHWQQQQVVI\WJY\LOLJDJD
OP P OYJ IOJFFJDYTHVTFP OJOIIJ IOOYIIDIHIDDDIZ J \ VKNGHYLNHYTHI\NGW\QNONWNGH
STUHWONDLK\DOQFFJOWUTKGHDYGDQVODHDNYODQUHOGN\JYVGI\NUOLQNDNWYHJYHDON
OKLODDOSWUDJJYHTILVGLFSNNGYOHWIWYNVFSGDLNHYIGQNIKHJDYJLJLDYYPLIJPLIVPLO
FFDLUUQUHPYJVYIWOHGIYJGZ UT WDJ \ QOGTYOHT J JYVVOIT QOJYVYWSITUIYOVJHQJ ON
LGLKYLLS\HJOVJGTPJTLHNLINYY\SYGGKKINYIOK\JWOYIGJYWSQPLG\IJUS\HJLDYIGJNN
IWYWJWOZ QJQNILGHUOLQSGJVOOIUYWLQJYWJZ UOFHULOD#

SEQ ID NO:23 hCD63-Nluc (SEQ ID NO: 56)

P DYHJJPNFYNIOO\YOOODIFDFDYJOLDYJYJDTOYOVTWJWLLTJDWSJVOOSYYLLDYJYIOIOYD
IYJ FFJDFNHQ\FOP IWIDLIOVOIP OYHYDDDLDJ \YIUGNYPVHIQQQ IUTT P HQ\SNQQKWDVIO
GUPTDGINFFJDJVDQ \ WGZ HNLSVP VNQUYSGVFFLQYWYJFJIQIQLKNDLKNHJFYHNLJJZ OU
NQYOYYDDDDOJLDIYHYOJLYIDFFOYNVLUVJ\HYPYIWOHGIYJGZ UTWDJ \ QOGTYOHTJJYVV
OITQOJYVYWSLTULYOVJHQJONLGLKYLLS \HJOVJGTPJTLHNLINYY\ SYGGKKINYLOK \JWOYLG
JYWSQP IG\IJUS\HJLDYIGJNNLWYWJWOZ QJQNLLGHUOLQSGJVOOIUYWLQJYWJZ UOFHULOD#

SEQ ID NO:24 hCD63-ABDX2-Nluc (SEQ ID NO: 57)

P DYHJJPNFYNIOO\ YOOODIFDFDYJOLDYJYJOTOYOVTWJWODHDNYODQUHOGN\JYVGI\NUO
LQNDNWYHJYHDONOKLODDOSJ WLLT JDWSJVOOSYYLLDYJYIOIOYDIYJFFJDFNHQ \ FOP IWIDIIO
VOIP OYHYDDDLDJ \YIUGNYPVHIQQQIUTTPHQ \SNQQKWDVLOGUPTDGINFFJDJVTKGHDY
GDQVODHDNYODQUHOGN\JYVGI\NUOLQNDNWYHJYHDONOKLODDOSJVDQ\WGZ HNLSVP VNQU
YSGVFFLQYWYJFJLQIQHNDIKNHJFYHNLJJZ OUNQYOYYDDDDOJLDIYHYOJLYIDFFOYNVLUVJ
\HYPYIWOHGIYJGZ UTWDJ \ QOGTYOHTJJYVVOITQOJYVYWSLTULYOVJHQJONLGIKYLLS\HJ
OVJGTP JTLHNLINYY\SYGGKKINYLOK\JWOYLGJYWSQP LG \ IJUS \HJLDYIGJNNLWYWJWOZ Q
J QNLLGHUOLQSGJVOOIUYWLQJYWJZ UOFHULOD#

SEQ ID NO:25 hCD63-ABD 1 st-Nluc (SEQ ID NO: 58)

P DYHJJPNFYNIOO\ YOOODIFDFDYJOLDYJYJDTOYOVTWJWODHDNYODQUHOGN\JYVGI\NUO
LQNDN4VYHJYHDONOKIODDOSJ WLLT JDWSJVOOSYYLLDYJYIOIOYDIYJFFJDFNHQ \ FOP IWIDIIO
VOIPOYHYDDDLDJ\YIUGNYPVHIQQQIUTTPHQ \SNQQKWDVLOGUPTDGINFFJDJVDQ \ WGZ
HNLSVP VNQUYSGVFFLQYWYJFJLQIQHNDIKNHJFYHNLJJZ OUNQYOYYDDDDOJLDIYHYOJIYI
DFFOYNVLUVJ\HYPYIWOHGIYJGZ UTWDJ \QOGTYOHTJJYVVOITQOJYVYWSLTULYOVJHQJ
ONLGLKYLLS\HJOVJGTPJTLHNLINYY\SYGGKKINYLOK\JWOYLGJYWSQPLG\IJUS\HJLDYIGJ
NNLWYWJWOZ QJQNILGHUOIQSGJVOOIUYWIQJYWJZ UOFHUIOD#

SEQ ID NO:26 hCD63-ABD 2 nd-Nluc (SEQ ID NO: 59)

P DYHJJPNFYNIOO\YOOODIFDFDYJOLDYJYJDTOYOVTWJWLLTJDWSJVOOSYYLLDYJYIOIOYD
IYJ FFJDFNHQ\FOP IWIDLIOVOIP OYHYDDDLDJ \YIUGNYPVHIQQQ IUTT PHQ\SNQQK4VDVLO
GUPTDGINFFJDJVTKGHDYGDQVODHDNYODQUHOGN\JYVGI\NUOIQNDNWYHJYHDONOKLOD
DOSJVDQ \ WGZ HNLSVPVNQUYSGVFFLQYWYJFJLQIQHNDLKNHJFYHNLJJZ OUNQYOYYDDDDO
JLDIYHYOJLYIDFFOYNVLUVJ\HYPYIWOHGIYJGZ UTWDJ \QOGTYOHTJJYVVOITQOJYVYW
SLTULYOVJHQJONIGLKYLLS \ HJOVJGTPJTLHNLINYY\SYGGKKINYIOK \ JWOYLGJYWSQPLG \ I
JUS\HJLDYIGJNNIWYWJWOZ QJQNILGHUOIQSGJVOOIUYWIQJYWJZ UOFHUIOD#

SEQ ID NO:27 hCD63-RC 2 nd-Nluc (SEQ ID NO: 60)

P DYHJJPNFYNIOO\YOOODIFDFDYJOLDYJYJDTOYOVTWJWLLTJDWSJVOOSYYLLDYJYIOIOYD
IYJ FFJDFNHQ\FOP IWIDLIOVOIP OYHYDDDLDJ \YIUGNYPVHIQQQ IUTT P HQ\SNQQKWDVLO
GUP TDGINFFJDJVJNJFN\YNONFIKSIGVIJOLQNSILNYWQSLOINISLVT \ IVIJTDYVL\ VOIP O
JVDQ \ WGZ HNLSVPVNQUYSGVFFLQYWYJFJIQIQHNDLKNHJFYHNLJJZ OUNQYOYYDDDDOJLD
IYHYOJLYIDFFOYNVLUVJ\HYPYIWOHGIYJGZ UTWDJ \QOGTYOHTJJYVVOITQOJYVYWSLT
ULYOVJHQJONLGIKYLLS \ HJOVJGTPJTLHNLINYY \ SYGGKKINYLOK \ JWOYLGJYWSQPLG \ IJUS
\HJLDYIGJNNLWYWJWOZ QJQNILGHUOIQSGJVOOIUYWIQJYWJZ UOFHUIOD#

SEQ ID NO:28 hCD81-Nluc (SEQ ID NO: 61)

P JYHJFWNFLN\OOIYIQIYIZ ODJJYLOJYDOZ OUKGSTWWQOO\OHP KOJ GNSDSQWI\YJL\LOLD
YJDYP P IYJ IOJF\JDLTHVTFOOJWIIWFOYIDIDFHYDDJLZ J IYQNGTILDNGYNT I\GTDOTTD
YYGGGDQQDNDYYNWIKHWOG FFJVWUVWOWDOWWVYONQQOF SVJVQLLVQOINHG FKTNLGGOIVJ N
O\OLJLDDLYYDYLP LIHP LOVP YOFFJLUQVVY\JVYIWOHGIYJGZ UTWDJ \ QOGTYOHTJJYVVOI
TQOJYVYWSLTULYOVJHQJONLGLKYLLS \HJOVJGTPJTILHNLINYY\SYGGKKINYLOK \JWOYLGJY
WSQP LG \IJUS\HJLDYIGJNNLWYWJWOZ QJQNLLGHUOLQSGJVOOIUYWLQJYWJZ UOFHUIOD#

SEQ ID NO:29 hCD81-ABDX2-Nluc (SEQ ID NO: 62)

PJYHJFWNFLN\OOIYIQIYIZ ODJJYLOJYDOZ OUKGSTWWQOO\OHP KODHDNYODQUHOGN\JYV
GI\NUOLQNDNWYHJYHDONOKLODDOSPKOJGNSDSQWI\YJL\LOLDYJDYPP IYJIOJF\JDLTHVT
FOOJWIIWFOYLOIDFHYDDJLZ J IYQNGTLDNGYNT I\GTDOTTDYYGGGDQQDNDYYNWIKHWOGF
FJVWUTKGHDYGDQVODHDNYODQUHOGN\JYVGI\NUOLQNDNWYHJYHDONOKLODDOSWUVWOWDO
WWVYONQQOFSVJVQLLVQOINHGFKTNLGGOIVJNO\OLJLDDLYYDYLPIIHPLOVPYOFFJQVVY\J
VYIWOHGIYJGZ UTWDJ\QOGTYOHTJJYVVOITQOJYVYWSLTULYOVJHQJONLGLKYLLS\HJOVJ
GTPJTLHNLINYY\SYGGKKINYLOK\JWOYLGJYWSQPLG\IJUS\HJLDYIGJNNLWYWJWOZ QJQN
LLGHUOLQSGJVOOIUYWLQJYWJZ UOFHUIOD#

SEQ ID NO:30 hCD81-ABD 1 st-Nluc (SEQ ID NO: 63)

PJYHJFWNFLN\OOIYIQIYIZ ODJJYLOJYDOZ OUKGSTWWQOO\OHP KODHDNYODQUHOGN\JYV
GI\NUOLQNDNWYHJYHDONOKLODDOSP KOJGNSDSQWI\YJL\LOLDYJDYP P IYJ IOJF\JDITHVT
FOOJWIIWFOYLOIDFHYDDJLZ J IYQNGTLDNGYNT I\GTDOTTDYYGGGDQQDNDYYNWIKHWOGF
FJVWUVWOWDOWWVYONQQOFSVJVQLLVQOINHGFKTNLGGOIVJNO\OLJLDDLYYDYLP LIHP LOVP YO
FFJLUQVVY\JVYIWOHGIYJGZ UTWDJ\QOGTYOHTJJYVVOITQOJYVYWSLTULYOVJHQJONL
GLKYLLS\HJOVJGTPJTLHNLINYY\SYGGKKINYLOK\JWOYLGJYWSQPLG\IJUS\HJLDYIGJNNL
WYWJWOZ QJQNILGHUOIQSGJVOOIUYWIQJYWJZ UOFHULOD#

SEQ ID NO:31 hCD81-ABD 2 nd-Nluc (SEQ ID NO: 64)

P JYHJFWNFIN\OOIYIQIYIZ ODJJYIOJYDOZ OUKGSTWWQOO\OHP KOJGNSDSQWI\YJI\IOID
YJDYP P IYJ IOJF\JDPTHVTFOOJWIIWFOYIOIDFHYDDJIZ J IYQNGTKINGYNT I\GTDOTTD
YYGGGDQQDNDYYNWIKHWOGFFJVWUTKGHDYGDQVODHDNYODQUHOGN\JYVGI\NUOIQNDNW
YHJYHDONOKIODDOSWUVWOWDOWWVYONQQOFSVJVQHVQOINHGFKTNIGGOIVJNO\OUIDDIYYD
YIPIIHPIDVPYOFFJLUQVVY\JVYIWOHGIYJGZ UTWDJ\QOGTYOHTJJYVVOITQOJYVYWSET
UIYOVJHQJONIGIKYIIS\HJOVJGTPJTIHNIINYY\SYGGKKINYIOK\J WOYIGJYWSQPIG\IJUS
\HJIDYIGJNNIWYWJWOZ QJQNUGHUOIQSGJVOOIUYWIQJYWJZ UOFHUIOD#

SEQ ID NO:32 Lamp2B-Nluc (SEQ ID NO: 65)

P YF IUOISYSJVJOYOYFOYOJDYUV\DONIHWJJDSP KIVTDYKDDKDHIQHDJUVUQIHNOFWVY\
GIIYZ OJUJKOOJUODDIYJNTYOOJUNYYYYUVKFKZ QGODYISDJYYKQZ GIHSUNYVSVNSVI
THIYGZ HQYVSHOQVWGTSIOFWJVJVJVJVJVONSDJIOISIIYJDJOVJOHYIYID\YUUUNV\DJD
TWOJJJJV\ISHDSUGJTD\YUNGJHZ YOOVWIOVSDQJWSWSWSWSWJUVO\SVOHGONYGNYITD
TWDIVDQSDQSDIOVHDVDSISKGJQO\SUO\SHOVT\PJOVOHJJTOJWSWSWSWSWJJVYIWOHGI
YJGZ UTWDJ\QOGTYOHTJJYVVOITQOJYVYWSETUIYOVJHQJONIGIKYIIS\HJOVJGTPJTIH
NIINYY\SYGGKKINYIDK\JWOYIGJYWSQPIG\IJUS\HJIDYIGJNNIWYWJWOZ QJQNUGHUOIQS
GJVOOIUYWIQJYWJZ UOFHUIOD#

SEQ ID NO:33 Lamp2B-ABD-Nluc (SEQ ID NO: 66)

P YF IUOISYSJ VJ OYOYFOYOJDYUV\DONIHT KGHDYGDQVODHDNYODQUHOGN\JYVG I\NUOIQ
NDNWYHJYHDONOKIDDDOSIHWJJDSP KIVTDYKDDKDHIQHDJUVUQIHNOFWVY\GIIYZ OJUJK
OOJUODDIYJNTYOOJUNYYYYUVKFKZ QGODYISDJYYKQZ GIHSUNYVSVNSVITHIYGZ HQYV
SHOQVWGTSIOFWJVJVJVJVJVONSDJIOISHYJDJOVJOHYIYID\YUUUNV\DJDTWOJJJJV\L
SHDSUGJTD\YUNGJHZ YOOVWIOVSDQJWSWSWSWSWJUVO\SVOHGONYGNYITDTWDIVDQSDQ
SDIOVHDVDSISKGJQO\SUO\SHOVT\PJOVOHJJTOJWSWSWSWSWJJVYIWOHGIYJGZ UTWDJ
\QOGTYOHTJJYVVOITQOJYVYWSETUIYOVJHQJONIGIKYIIS\HJOVJGTPJTIHNIINYY\SYGG
KKINYIOK\J WOYIGJYWSQPIG\IJUS\HJIDYIGJNNIWYWJWOZ QJQNIIGHUOIQSGJVOOIUYWIQ
JYWJZ UOFHUIOD#

1. An extracellular vesicle (EV) modified to comprise at least one albumin binding domain (ABD) present on the surface of the EV.

2. An EV according to claim 1, wherein the ABD forms part of a fusion protein with an EV protein, optionally wherein the EV protein is a transmembrane EV protein or an EV protein associated with the outer surface of the EV membrane.

3. An EV according to claim 2, wherein the ABD is engineered into an extravesicular loop of a multi-pass transmembrane protein, optionally wherein the multi-pass transmembrane protein is a tetraspanin.

4. An EV according to claim 2, wherein the EV protein is selected from the group consisting of: CD63, CD81, CD9, CD82, CD44, CD47, CD55, LAMP2B, ICAMs and ARRDC1, as well as derivatives, domains, variants, mutants, or regions thereof.

5. An EV according to claim 1, wherein the EV comprises more than one ABD.

6. An EV according to claim 2, wherein more than one ABD is present in the same fusion protein.

7. An EV according to claim 1, wherein the EV is further loaded with a therapeutic cargo, optionally wherein the therapeutic cargo is a protein, nucleic acid, virus, viral genome, antigen or small molecule.

8. An EV according to claim 7, wherein the therapeutic cargo is selected from the group consisting of: a nucleic acid, optionally an RNA molecule, a DNA molecule or a mixmer, mRNA, an antisense or splice-switching oligonucleotide, siRNA, shRNA, miRNA, plasmid DNA (pDNA), a supercoiled or unsupercoiled plasmid, or a mini-circle; a peptide or protein, optionally a transporter, enzyme, receptor (optionally a decoy receptor), membrane protein, cytokine, antigen or neoantigen, ribonuclear protein, nucleic acid binding protein, antibody, nanobody or an antibody fragment; an antibody-drug conjugate; a small molecule drug; gene editing technology, optionally CRISPR-Cas9, a TALEN, meganuclease; or a vesicle-based cargo, optionally a virus, optionally an AAV or a lentivirus.

9. An EV according to claim 7, wherein the therapeutic cargo is present on the inside of the EV, on the outside of the EV and/or in the membrane of the EV.

10. An EV according to claim 7, wherein the therapeutic cargo forms part of the EV protein-ABD fusion protein.

11. An EV according to claim 1, wherein the EV further comprises a targeting moiety.

12. An EV according to claim 11, wherein the targeting moiety forms part of the ABD fusion protein.

13. A method for producing an EV according to claim 1, comprising: (i) introducing into an EV-producing cell at least one polynucleotide construct encoding an ABD-EV protein fusion construct; and (ii) expressing said construct in the EV-producing cell, thereby generating an EV comprising ABD present on the surface of the EV.

14. A pharmaceutical composition comprising at least one EV according to claim 1 and a pharmaceutically acceptable excipient or carrier.

15. A method of treating a disease in a patient comprising administering to the patient an effective amount of an EV according to claim 1.

16. A method of treating a disease in a patient, comprising administering to the patient an effective amount of the pharmaceutical composition according to claim 14.
